SEQUENCE LISTING (52 sequences)

SEQ ID NO: 1 - protein, 5 aa, synthetic construct
SYAMN

SEQ ID NO: 2 - protein, 19 aa, synthetic construct
RIRSKYNNYATYYADSVKG

SEQ ID NO: 3 - protein, 14 aa, synthetic construct
ASNFPASYVSYFAY

SEQ ID NO: 4 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 5 - protein, 14 aa, synthetic construct
GSSTGAVTTSNYAN

SEQ ID NO: 6 - protein, 7 aa, synthetic construct
GTNKRAP

SEQ ID NO: 7 - protein, 9 aa, synthetic construct
ALWYSNLWV

SEQ ID NO: 8 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 19 aa, synthetic construct
RVRTTYNNREPYYADSVKG

SEQ ID NO: 10 - protein, 125 aa, synthetic construct
EVQLLESGGGLVQPGGSLRLSCAASGFTFSSYAMNWVRQAPGKGLEWVGRVRTTYNNREPYYADSVKGRFTISRDDSKNTLYLQMNSLRAEDTAVYYCVRASNFPASYVSYFAYWGQGTLVTVSS

SEQ ID NO: 11 - protein, 7 aa, synthetic construct
GTNKLAP

SEQ ID NO: 12 - protein, 109 aa, synthetic construct
QAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWVFGGGTKLTVL

SEQ ID NO: 13 - protein, 19 aa, synthetic construct
RVRTKYNNREPYYADSVKG

SEQ ID NO: 14 - protein, 125 aa, synthetic construct
EVQLLESGGGLVQPGGSLRLSCAASGFTFSSYAMNWVRQAPGKGLEWVGRVRTKYNNREPYYADSVKGRFTISRDDSKNTLYLQMNSLRAEDTAVYYCVRASNFPASYVSYFAYWGQGTLVTVSS

SEQ ID NO: 15 - protein, 19 aa, synthetic construct
RQRTQYNNREDYYADSVKG

SEQ ID NO: 16 - protein, 125 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRASNFPASYVSYFAYWGQGTLVTVSS

SEQ ID NO: 17 - protein, 125 aa, synthetic construct
EVQLLESGGGLVQPGGSLRLSCAASGFTFSSYAMNWVRQAPGKGLEWVGRVRTTYNNREPYYADSVKGRFTISRDDSKNTLYLQMNSLRAEDTAVYYCVRASNFPASYVSYFAYWGQGTSVTVSG

SEQ ID NO: 18 - protein, 125 aa, synthetic construct
EVQLLESGGGLVQPGGSLRLSCAASGFTFSSYAMNWVRQAPGKGLEWVGRVRTKYNNREPYYADSVKGRFTISRDDSKNTLYLQMNSLRAEDTAVYYCVRASNFPASYVSYFAYWGQGTSVTVSG

SEQ ID NO: 19 - protein, 125 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRASNFPASYVSYFAYWGQGTSVTVSG

SEQ ID NO: 20 - protein, 5 aa, synthetic construct
DTYIH

SEQ ID NO: 21 - protein, 17 aa, synthetic construct
RIYPTNGYTRYADSVKG

SEQ ID NO: 22 - protein, 11 aa, synthetic construct
WGGEGFYAMDY

SEQ ID NO: 23 - protein, 120 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSS

SEQ ID NO: 24 - protein, 11 aa, synthetic construct
RASQDVNTAVA

SEQ ID NO: 25 - protein, 7 aa, synthetic construct
SASFLYS

SEQ ID NO: 26 - protein, 9 aa, synthetic construct
QQHYTTPPT

SEQ ID NO: 27 - protein, 107 aa, synthetic construct [sequence omitted]

SEQ ID NO: 28 - protein, 671 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 29 - protein, 448 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 30 - protein, 232 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 232 aa, synthetic construct
EVQLLESGGGLVQPGGSLRLSCAASGFTFSSYAMNWVRQAPGKGLEWVGRVRTTYNNREPYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 232 aa, synthetic construct
EVQLLESGGGLVQPGGSLRLSCAASGFTFSSYAMNWVRQAPGKGLEWVGRVRTKYNNREPYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 232 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 232 aa, synthetic construct
EVQLLESGGGLVQPGGSLRLSCAASGFTFSSYAMNWVRQAPGKGLEWVGRQRTQYNNREDYYADSVKGRFTISRDDSKNTLYLQMNSLRAEDTAVYYCVRASNFPASYVSYFAYWGQGTSVTVSGASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 37 - protein, 177 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 38 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 39 - protein, 107 aa, Homo sapiens [sequence omitted]

SEQ ID NO: 40 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 42 - protein, 10 aa, synthetic construct
GGGGSGGGGS

SEQ ID NO: 43 - protein, 11 aa, synthetic construct
DGGGGSGGGGS

SEQ ID NO: 44 - protein, 10 aa, synthetic construct
GGGGSGGGGG

SEQ ID NO: 45 - protein, 11 aa, synthetic construct
DGGGGSGGGGG

SEQ ID NO: 46 - protein, 671 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGQAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 47 - protein, 671 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGQAVVTQEPSLTVSPGGTVTLTCGSSTGAVTTSNYANWVQEKPGQPPRGLIGGTNKLAPGTPARFSGSLLGGKAALTLSGAQPEDEAEYYCALWYSNLWVFGGGTKLTVLSEAEKKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 9 aa, synthetic construct
GGGGSGGGG

SEQ ID NO: 49 - protein, 360 aa, synthetic construct [sequence omitted]

SEQ ID NO: 50 - protein, 325 aa, synthetic construct
FKIPIEELEDRVFVNCNTSITWVEGTVGTLLSDITRLDLGKRILDPRGIYRCNGTDIYKDKESTVQVHYRMCRSEQLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 51 - protein, 351 aa, synthetic construct
QDGNEEMGSITQTPYQVSISGTTVILTCSQHLGSEAQWQHNGKNKEDSGDRLFLPEFSEMEQSGYYVCYPRGSNPEDASHHLYLKARVSENCVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 52 - protein, 334 aa, synthetic construct
FKIPVEELEDRVFVKCNTSVTWVEGTVGTLLTNNTRLDLGKRILDPRGIYRCNGTDIYKDKESAVQVHYRMSQNCVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

(19) United States
(12) Patent Application Publication
(10) Pub. No.: US 2025/0092136 A1
(43) Pub. Date: Mar. 20, 2025

(54) ANTIBODIES BINDING TO CD3

(71) Applicant: Hoffmann-La Roche Inc., Little Falls, NJ (US)

(72) Inventors: Alexander Christian BUJOTZEK, München (DE); Christian Robert GASSNER, Penzberg (DE); Christian KLEIN, Bonstetten (CH); Stéphane Gérard Alain LECLAIR, Gauting (DE); Stephan MAERSCH, Pähl (DE); Ekkehard MOESSNER, Kreuzlingen (CH); Inja WALDHAUER, Zürich (CH)

(73) Assignee: Hoffmann-La Roche Inc., Little Falls, NJ (US)

(21) Appl. No.: 18/784,587

(22) Filed: Jul. 25, 2024

(30) Foreign Application Priority Data
Jul. 26, 2023 (EP) 23187733.3

(51) Int. Cl. C07K 16/28; C07K 16/32

(52) U.S. Cl. CPC C07K 16/2809; C07K 16/32; C07K 2317/31; C07K 2317/52; C07K 2317/55; C07K 2317/565; C07K 2317/92

(57) ABSTRACT

The present invention generally relates to antibodies that bind to CD3, including multispecific antibodies e.g. for activating T cells. In addition, the present invention relates to polynucleotides encoding such antibodies, and vectors and host cells comprising such polynucleotides. The invention further relates to methods for producing the antibodies, and to methods of using them in the treatment of disease.

### CROSS-REFERENCE TO RELATED APPLICATIONS

This application claims priority under 35 U.S.C. 119 (b) to European Patent Application No. 23187733.3, filed Jul. 26, 2023, which is incorporated herein by reference in its entirety.

### SEQUENCE LISTING

The instant application contains a sequence listing which has been submitted electronically in .xml format and is hereby incorporated by reference in its entirety. Said .xml copy, created on Jul. 10, 2024, is named P38530-US-Sequence Listing .xml and is 53,455 bytes in size.

### FIELD OF THE INVENTION

The present invention generally relates to antibodies that bind to CD3, including multispecific antibodies e.g. for activating T cells. In addition, the present invention relates to polynucleotides encoding such antibodies, and vectors and host cells comprising such polynucleotides. The invention further relates to methods for producing the antibodies, and to methods of using them in the treatment of disease.

### BACKGROUND

CD3 (cluster of differentiation 3) is a protein complex composed of four subunits, the CD3γ chain, the CD3δ chain, and two CD3ε chains. CD3 associates with the T-cell receptor and the ζ chain to generate an activation signal in T lymphocytes.

CD3 has been extensively explored as drug target. Monoclonal antibodies targeting CD3 have been used as immunosuppressant therapies in autoimmune diseases such as type I diabetes, or in the treatment of transplant rejection. The CD3 antibody muromonab-CD3 (OKT3) was the first monoclonal antibody ever approved for clinical use in humans, in 1985.

A more recent application of CD3 antibodies is in the form of bispecific antibodies, binding CD3 on the one hand and a tumor cell antigen on the other hand. The simultaneous binding of such an antibody to both of its targets will force a temporary interaction between target cell and T cell, causing activation of any cytotoxic T cell and subsequent lysis of the target cell.

Efficacy and safety are key requirements of antibodies for therapeutic purposes. These can be hampered, however, by liabilities such as immunogenicity, non-specific binding, suboptimal pharmacokinetic (PK) properties and the like.

Given the tremendous therapeutic potential of antibodies, particularly bispecific antibodies for the activation of T cells, there is a need for CD3 antibodies with optimized properties.

### SUMMARY OF THE INVENTION

The present inventors aimed at generating antibodies that bind to CD3, with optimized properties for therapeutic use, e.g. as bispecific antibodies in cancer immunotherapy.

High affinity binding to CD3 was not considered a priority. Rather to the contrary, the inventors aimed at modulating binding affinity to be in an intermediate to low range, in order to reduce the potential for adverse events such as excessive cytokine release induced by the antibodies (cytokine release syndrome (CRS) being a severe and common side effect of CD3 antibodies, in particular CD3 bispecific antibodies as used in cancer immunotherapy).

The inventors also aimed at minimizing immunogenicity of the antibodies, in order to minimize the risk of anti-drug antibodies (ADA) being formed against them, which can interfere with therapeutic efficacy or even pose a safety risk in patients.

Further, the inventors aimed at antibodies that bind to CD3-expressing cells with high specificity, to prevent undesired effects on non-target cells. The reduction of non-specific binding to cells and/or cellular accumulation (e.g. due to a reduction in positively charged patches and hydrophobic, surface-exposed residues) also serves to reduce non-specific clearance and thereby improve pharmacokinetic properties and may also contribute to reducing the risk of ADA formation.

At the same time, of course, the CD3 antibodies should show sufficient functional activity for therapeutic efficacy, and preserve desirable properties like produceability and stability.

The invention accordingly provides antibodies that bind to CD3 which combine the above-mentioned favorable properties for therapeutic use.

In one aspect, the invention provides an antibody that binds to CD3, wherein the antibody comprises a first antigen binding domain, comprising
(i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3,
and
(ii) a light chain variable region (VL) comprising a light chain complementarity determining region (LCDR) 1 of SEQ ID NO: 5, a LCDR 2 of SEQ ID NO: 11 and a LCDR 3 of SEQ ID NO: 7.

In one aspect, the VH of the first antigen binding domain comprises an amino acid sequence that is at least about 95%, 96%, 97%, 98%, 99% or 100% identical to an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and/or the VL of the first antigen binding domain comprises an amino acid sequence that is at least about 95%, 96%, 97%, 98%, 99% or 100% identical to the amino acid sequence of SEQ ID NO: 12.

In one aspect, the VH of the first antigen binding domain comprises an amino acid sequence selected from the group consisting of SEQ ID NO: 10, SEQ ID NO: 14 and SEQ ID NO: 16, and/or the VL sequence of the first antigen binding domain comprises the amino acid sequence of SEQ ID NO: 12.

In one aspect, the VH of the first antigen binding domain comprises amino acid substitutions L108S and S113G (Kabat numbering). Accordingly, in one aspect, the VH of the first antigen binding domain comprises an amino acid sequence selected from the group consisting of SEQ ID NO: 17, SEQ ID NO: 18 and SEQ ID NO: 19, and/or the VL sequence of the first antigen binding domain comprises the amino acid sequence of SEQ ID NO: 12.

In one aspect, the invention provides an antibody that binds to CD3, wherein the antibody comprises a first antigen binding domain comprising a VH sequence selected from the group consisting of SEQ ID NO: 10, SEQ ID NO: 14, SEQ ID NO: 16, SEQ ID NO: 17, SEQ ID NO: 18 and SEQ ID NO: 19, and/or a VL sequence of SEQ ID NO: 12. In one such aspect, the invention provides an antibody that binds to CD3, wherein the antibody comprises a first antigen binding domain comprising a VH sequence selected from the group consisting of SEQ ID NO: 10, SEQ ID NO: 14 and SEQ ID NO: 16, and/or a VL sequence of SEQ ID NO: 12. In a further such aspect, the invention provides an antibody that binds to CD3, wherein the antibody comprises a first antigen binding domain comprising a VH sequence selected from the group consisting of SEQ ID NO: 17, SEQ ID NO: 18 and SEQ ID NO: 19, and/or a VL sequence of SEQ ID NO: 12.

In one aspect, the first antigen binding domain is a Fab molecule.

In one aspect, the antibody comprises an Fc domain composed of a first and a second subunit.

In one aspect, the antibody comprises a second and optionally a third antigen binding domain that binds to a second antigen.

In one aspect the second and/or, where present, the third antigen binding domain is a Fab molecule.

In one aspect the first antigen binding domain is a Fab molecule wherein the variable domains VL and VH or the constant domains CL and CH1, particularly the variable domains VL and VH, of the Fab light chain and the Fab heavy chain are replaced by each other.

In one aspect the second and, where present, the third antigen binding domain is a conventional Fab molecule.

In one aspect, the second and, where present, the third antigen binding domain is a Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, the first and the second antigen binding domain are fused to each other, optionally via a peptide linker.

In one aspect, the first and the second antigen binding domain are each a Fab molecule and either (i) the second antigen binding domain is fused at the C-terminus of the Fab heavy chain to the N-terminus of the Fab heavy chain of the first antigen binding domain, or (ii) the first antigen binding domain is fused at the C-terminus of the Fab heavy chain to the N-terminus of the Fab heavy chain of the second antigen binding domain.

In one aspect, the first, the second and, where present, the third antigen binding domain are each a Fab molecule and the antibody comprises an Fc domain composed of a first and a second subunit; and wherein either (i) the second antigen binding domain is fused at the C-terminus of the Fab heavy chain to the N-terminus of the Fab heavy chain of the first antigen binding domain and the first antigen binding domain is fused at the C-terminus of the Fab heavy chain to the N-terminus of the first subunit of the Fc domain, or (ii) the first antigen binding domain is fused at the C-terminus of the Fab heavy chain to the N-terminus of the Fab heavy chain of the second antigen binding domain and the second antigen binding domain is fused at the C-terminus of the Fab heavy chain to the N-terminus of the first subunit of the Fc domain; and the third antigen binding domain, where present, is fused at the C-terminus of the Fab heavy chain to the N-terminus of the second subunit of the Fc domain.

In one aspect, the Fc domain is an IgG, particularly an IgG₁, Fc domain. In one aspect the Fc domain is a human Fc domain. In one aspect, the Fc comprises a modification promoting the association of the first and the second subunit of the Fc domain. In one aspect, the Fc domain comprises one or more amino acid substitution that reduces binding to an Fc receptor and/or effector function.

In one aspect, the second antigen is a target cell antigen, particularly a tumor cell antigen.

In one aspect, the second antigen is HER2. In one aspect, the second and, where present, the third antigen binding domain comprises a VH comprising a HCDR 1 of SEQ ID NO: 20, a HCDR 2 of SEQ ID NO: 21, and a HCDR 3 of SEQ ID NO: 22, and a VL comprising a LCDR 1 of SEQ ID NO: 24, a LCDR 2 of SEQ ID NO: 25 and a LCDR 3 of SEQ ID NO: 26. In one aspect, the second and, where present, the third antigen binding domain comprises a VH comprising an amino acid sequence that is at least about 95%, 96%, 97%, 98%, 99% or 100% identical to the amino acid sequence of SEQ ID NO: 23, and/or a VL comprising an amino acid sequence that is at least about 95%, 96%, 97%, 98%, 99% or 100% identical to the amino acid sequence of SEQ ID NO: 27.

According to a further aspect of the invention there is provided an isolated polynucleotide encoding an antibody of the invention, and a host cell comprising the isolated polynucleotide of the invention.

In another aspect is provided a method of producing an antibody that binds to CD3, comprising the steps of (a) culturing the host cell of the invention under conditions suitable for the expression of the antibody and optionally (b) recovering the antibody. The invention also encompasses an antibody that binds to CD3 produced by the method of the invention.

The invention further provides a pharmaceutical composition comprising the antibody of the invention and a pharmaceutically acceptable carrier.

Also encompassed by the invention are methods of using the antibody and pharmaceutical composition of the invention. In one aspect the invention provides an antibody or pharmaceutical composition according to the invention for use as a medicament. In one aspect is provided an antibody or pharmaceutical composition according to the invention for use in the treatment of a disease. In a specific aspect the disease is cancer.

Also provided is the use of an antibody or pharmaceutical composition according to the invention in the manufacture of a medicament, the use of an antibody or pharmaceutical composition according to the invention in the manufacture of a medicament for the treatment of a disease, particularly cancer. The invention also provides a method of treating a disease in an individual, comprising administering to said individual an effective amount of the antibody or pharmaceutical composition according to the invention.

### BRIEF DESCRIPTION OF THE DRAWINGS

FIGS. 1A-1Z. Exemplary configurations of the (multispecific) antibodies of the invention. (FIG. 1A, FIG. 1D) Illustration of the “1+1 CrossMab” molecule. (FIG. 1B, FIG. 1E) Illustration of the “2+1 IgG Crossfab” molecule with alternative order of Crossfab and Fab components (“inverted”). (FIG. 1C, FIG. 1F) Illustration of the “2+1 IgG Crossfab” molecule. (FIG. 1G, FIG. 1K) Illustration of the “1+1 IgG Crossfab” molecule with alternative order of Crossfab and Fab components (“inverted”). (FIG. 1H, FIG. 1L) Illustration of the “1+1 IgG Crossfab” molecule. (FIG. 1I, FIG. 1M) Illustration of the “2+1 IgG Crossfab” molecule with two CrossFabs. (FIG. 1J, FIG. 1N) Illustration of the “2+1 IgG Crossfab” molecule with two CrossFabs and alternative order of Crossfab and Fab components (“inverted”). (FIG. 1O, FIG. 1S) Illustration of the “Fab-Crossfab” molecule. (FIG. 1P, FIG. 1T) Illustration of the “Crossfab-Fab” molecule. (FIG. 1Q, FIG. 1U) Illustration of the “(Fab) 2-Crossfab” molecule. (FIG. 1R, FIG. 1V) Illustration of the “Crossfab-(Fab)₂” molecule. (FIG. 1W, FIG. 1Y) Illustration of the “Fab-(Crossfab)₂” molecule. (FIG. 1X, FIG. 1Z) Illustration of the “(Crossfab)₂-Fab” molecule. Black dot: optional modification in the Fc domain promoting heterodimerization. ++, −−: amino acids of opposite charges optionally introduced in the CH1 and CL domains. Crossfab molecules are depicted as comprising an exchange of VH and VL regions, but may—in aspects wherein no charge modifications are introduced in CH1 and CL domains—alternatively comprise an exchange of the CH1 and CL domains.

FIGS. 2A-2E. (FIG. 2A) Schematic illustration of the T-cell bispecific antibody (TCB) molecules used in the Examples. All tested TCB antibody molecules were produced as “2+1 IgG CrossFab, inverted” with charge modifications (VH/VL exchange in CD3 binder, charge modifications in target cell antigen binders, EE=147E, 213E; RK=123R, 124K). (B-E) Components for the assembly of the TCB: light chain of anti-target antigen Fab molecule with charge modifications in CH1 and CL (FIG. 2B), light chain of anti-CD3 crossover Fab molecule (FIG. 2C), heavy chain with knob and PG LALA mutations in Fc region (FIG. 2D), heavy chain with hole and PG LALA mutations in Fc region (FIG. 2E).

FIG. 3. Schematic illustration of the surface plasmon resonance (SPR) setup used in Example 3.

FIGS. 4A and 4B. Binding of HER2-TCBs containing the optimized CD3 binders in comparison to the parental binder PIAH4929/CD3_{orig} (FIG. 4A) and the optimized CD3 binders alone (FIG. 4B) to CD3-expressing Jurkat NFAT reporter cells measured by flow cytometry.

FIGS. 5A-5C. The TCBs containing optimized CD3 binders were tested in a Jurkat NFAT reporter cell assay with KPL-4 cells (FIG. 5A, FIG. 5B) or MKN-45 cells (FIG. 5C). Comparison was done to a TCB containing PIAH4929/CD3_{orig}. Activation of the Jurkat NFAT cells was determined by measuring luminescence after 5 h upon treatment.

FIGS. 6A and 6B. Tumor cell killing of HER2-expressing KPL-4 cells (FIG. 6A) and MKN-45 cells (FIG. 6B) with PBMCs from a healthy donor was assessed when treated with TCBs either containing the optimized CD3 binders or the parental binder PIAH4929/CD3_{orig}. Tumor cell killing was measured by quantification of LDH release after 48 h.

FIGS. 7A-7D. CD25 upregulation on CD8 T cells (FIG. 7A, FIG. 7C) and CD4 T cells (FIG. 7B, FIG. 7D) was analyzed in PBMCs from a healthy donor treated with TCBs either containing the optimized CD3 binders or the parental binder PIAH4929/CD3_{orig}, in presence of the HER2-expressing KPL-4 cells (FIG. 7A, FIG. 7B) or MKN-45 cells (FIG. 7C, FIG. 7D) as target cell lines. Analysis was done by flow cytometry after 48 h incubation.

FIG. 8. The amount of bound antibodies to CD3-negative CHO-K1 cells measured by cell ELISA (antibody concentration 200 nM).

FIGS. 9A and 9B. Relative nonspecific cellular accumulation rate of IgGs (FIG. 9A) or TCBs (FIG. 9B) in human primary liver sinusoidal endothelial cells, comparing the parental binder CD3_{orig} with the optimized CD3 binders.

### DETAILED DESCRIPTION OF THE INVENTION

### I. Definitions

Terms are used herein as generally used in the art, unless otherwise defined in the following.

As used herein, the terms “first”, “second” or “third” with respect to antigen binding domains etc., are used for convenience of distinguishing when there is more than one of each type of moiety. Use of these terms is not intended to confer a specific order or orientation of the moiety unless explicitly so stated.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one aspect, the extent of binding of an anti-CD3 antibody to an unrelated, non-CD3 protein is less than about 10% of the binding of the antibody to CD3 as measured, e.g., by surface plasmon resonance (SPR). In certain aspects, an antibody that binds to CD3 has a dissociation constant (K_{D}) of ≤1 μM, ≤500 nM, ≤ 200 nM, or ≤100 nM. An antibody is said to “specifically bind” to CD3 when the antibody has a K_{D} of 1 μM or less, as measured, e.g., by SPR. In certain aspects, an anti-CD3 antibody binds to an epitope of CD3 that is conserved among CD3 from different species.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv and scFab), single-domain antibodies, and multispecific antibodies formed from antibody fragments. For a review of certain antibody fragments, see Hollinger and Hudson, Nature Biotechnology 23:1126-1136 (2005).

The terms “full-length antibody,” “intact antibody,” and “whole antibody” are used herein interchangeably to refer to an antibody having a structure substantially similar to a native antibody structure.

The term “monoclonal antibody” as used herein refers to an antibody obtained from a population of substantially homogeneous antibodies, i.e. the individual antibodies comprised in the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC, affinity chromatography, size exclusion chromatography) methods. For review of methods for assessment of antibody purity, see, e.g., Flatman et al., J. Chromatogr. B 848:79-87 (2007). In some aspects, the antibodies provided by the present invention are isolated antibodies.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. Such variable domains are referred to herein as “humanized variable region”. A humanized antibody optionally may comprise at least a portion of an antibody constant region derived from a human antibody. In some aspects, some FR residues in a humanized antibody are substituted with corresponding residues from a non-human antibody (e.g., the antibody from which the CDR residues are derived), e.g., to restore or improve antibody specificity or affinity. A “humanized form” of an antibody, e.g. of a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In certain aspects, a human antibody is derived from a non-human transgenic mammal, for example a mouse, a rat, or a rabbit. In certain aspects, a human antibody is derived from a hybridoma cell line. Antibodies or antibody fragments isolated from human antibody libraries are also considered human antibodies or human antibody fragments herein.

The term “antigen binding domain” refers to the part of an antibody that comprises the area which binds to and is complementary to part or all of an antigen. An antigen binding domain may be provided by, for example, one or more antibody variable domains (also called antibody variable regions). In preferred aspects, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and complementarity determining regions (CDRs). See, e.g., Kindt et al., Kuby Immunology, 6^{th} ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

Glutamine or glutamate residues at the N-terminus of antibody heavy or light chains may be converted to pyro-glutamate spontaneously (see e.g. Liu et al., Journal of Pharmaceutical Sciences 97, 2426-2447 (2008), Rehder et al., Journal of Chromatography A 1102, 164-175 (2006), Chelius et al., Anal Chem 78, 2370-2376 (2006)). Hence, variable regions or variable domains disclosed herein which comprise either a glutamine (Q) or a glutamate (E) amino acid residue at the N-terminus of an the antibody heavy or light chain, may comprise an N-terminal pyro-glutamate (pyroE) residue instead of the N-terminal Q or E residue. Likewise, antibody heavy chains or light chains disclosed herein which comprise either a glutamine (Q) or a glutamate (E) amino acid residue at the N-terminus, may comprise an N terminal pyro-glutamate (pyroE) residue instead of the N-terminal Q or E residue. Accordingly, for each antibody heavy chain, light chain, or variable domain or region sequence disclosed herein that contains an N-terminal Q or E residue, the corresponding sequence with an N-terminal pyroE residue is also encompassed.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, hinge, CH2 and CH3), which is herein further clarified by referring to “numbering according to Kabat EU index” or “Kabat EU index numbering” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementarity determining regions (CDRs). The FR of a variable domain generally consists of four FR domains: FR1, FR2, FR3, and FR4. Accordingly, the CDR and FR sequences generally appear in the following order in VH (or VL): FR1-HCDR1 (LCDR1)-FR2-HCDR2 (LCDR2)-FR3-HCDR3 (LCDR3)-FR4.

Unless otherwise indicated, CDR residues and other residues in the variable domain (e.g., FR residues) are numbered herein according to Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes is 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “Fc domain” or “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc region may or may not be present. Amino acid sequences of heavy chains including an Fc region (or a subunit of an Fc domain as defined herein) are denoted herein without C-terminal glycine-lysine dipeptide if not indicated otherwise. In one aspect, a heavy chain including an Fc region (subunit) as specified herein, comprised in an antibody according to the invention, comprises an additional C-terminal glycine-lysine dipeptide (G446 and K447, numbering according to Kabat EU index). In one aspect, a heavy chain including an Fc region (subunit) as specified herein, comprised in an antibody according to the invention, comprises an additional C-terminal glycine residue (G446, numbering according to Kabat EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

By “fused” is meant that the components (e.g. a Fab molecule and an Fc domain subunit) are linked by peptide bonds, either directly or via one or more peptide linkers.

The term “multispecific” means that the antibody is able to specifically bind to at least two distinct antigenic determinants. A multispecific antibody can be, for example, a bispecific antibody. Typically, a bispecific antibody comprises two antigen binding sites, each of which is specific for a different antigenic determinant. In certain aspects the multispecific (e.g. bispecific) antibody is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigenic determinant” or “antigen” refers to a site (e.g. a contiguous stretch of amino acids or a conformational configuration made up of different regions of non-contiguous amino acids) on a polypeptide macromolecule to which an antigen binding domain binds, forming an antigen binding domain-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). In a preferred aspect, the antigen is a human protein.

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one aspect, CD3 is human CD3, particularly the epsilon subunit of human CD3 (CD38). The amino acid sequence of human CD38 is shown in SEQ ID NO: 36 (without signal peptide). See also UniProt (www.uniprot.org) accession no. P07766 (entry version 224), or NCBI (www.ncbi.nlm.nih.gov/) RefSeq NP_000724.1. In another aspect, CD3 is cynomolgus (Macaca fascicularis) CD3, particularly cynomolgus CD38. The amino acid sequence of cynomolgus CD38 is shown in SEQ ID NO: 37 (without signal peptide). See also NCBI GenBank no. BAB71849.1. In certain aspects the antibody of the invention binds to an epitope of CD3 that is conserved among the CD3 antigens from different species, particularly human and cynomolgus CD3. In preferred aspects, the antibody binds to human CD3.

A “target cell antigen” as used herein refers to an antigenic determinant presented on the surface of a target cell, for example a cell in a tumor such as a cancer cell or a cell of the tumor stroma (in that case a “tumor cell antigen”). Preferably, the target cell antigen is not CD3, and/or is expressed on a different cell than CD3. In one aspect, the target cell antigen is HER2, particularly human HER2.

“HER2” or “ERBB2” stands for receptor tyrosine kinase erbB2 (also known as tyrosine kinase-type cell surface receptor HER2) and refers to any native HER2 from any vertebrate source, including mammals such as primates (e.g. humans), non-human primates (e.g. cynomolgus monkeys) and rodents (e.g. mice and rats), unless otherwise indicated. The term encompasses “full-length,” unprocessed HER2 as well as any form of HER2 that results from processing in the cell. The term also encompasses naturally occurring variants of HER2, e.g., splice variants or allelic variants. In one aspect, HER2 is human HER2. See for the human protein UniProt (www.uniprot.org) accession no. P04626 (entry version 268), or NCBI (www.ncbi.nlm.nih.gov/) RefSeq NP_004439.2. In certain aspects, the antibody of the invention binds to an epitope of HER2 that is conserved among the HER2 antigens from different species, particularly human and cynomolgus HER2. In preferred aspects, the antibody binds to human HER2.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (K_{D}). Affinity can be measured by well-established methods known in the art, including those described herein. A preferred method for measuring affinity is Surface Plasmon Resonance (SPR).

An “affinity matured” antibody refers to an antibody with one or more alterations in one or more complementarity determining regions (CDRs), compared to a parent antibody which does not possess such alterations, such alterations resulting in an improvement in the affinity of the antibody for antigen.

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. Suitable assays to measure T cell activation are known in the art and described herein.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In some aspects, the modification promoting the association of the first and the second subunit of the Fc domain comprises an amino acid mutation in the Fc domain, specifically an amino acid substitution. In a preferred aspect, the modification promoting the association of the first and the second subunit of the Fc domain comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-258), and Pearson et. al. (Genomics 46 (1997) 24-36) and is publicly available from www.fasta.bioch.virginia.cdu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “polynucleotide” or “nucleic acid molecule” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al. (2017) Nature Medicine 23:815-817, or EP 2 101 823 B1).

An “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that is different from its natural chromosomal location.

“Isolated polynucleotide (or nucleic acid) encoding an antibody” refers to one or more polynucleotide molecules encoding antibody heavy and light chains (or fragments thereof), including such polynucleotide molecule(s) in a single vector or separate vectors, and such polynucleotide molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antibodies of the present invention. Host cells include cultured cells, e.g. mammalian cultured cells, such as HEK cells, CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. In one aspect, the host cell of the invention is a eukaryotic cell, particularly a mammalian cell. In one aspect, the host cell is not a cell within a human body.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human.

An “effective amount” of an agent, e.g., a pharmaceutical composition, refers to an amount effective, at dosages and for periods of time necessary, to achieve the desired therapeutic or prophylactic result.

The term “package insert” is used to refer to instructions customarily included in commercial packages of therapeutic products, that contain information about the indications, usage, dosage, administration, combination therapy, contraindications and/or warnings concerning the use of such therapeutic products.

### II. Compositions and Methods

The invention provides antibodies that bind CD3, including multispecific antibodies that bind CD3 and a second antigen. The antibodies show reduced immunogenicity risk, non-specific binding to cells and cellular accumulation, combined with other favorable properties for therapeutic application, e.g. with respect to efficacy and safety, pharmacokinetics, as well as produceability. Antibodies of the invention as useful, e.g., for the treatment of diseases such as cancer.

### A. Anti-CD3 Antibodies

In one aspect, the invention provides antibodies that bind to CD3. In one aspect, provided are isolated antibodies that bind to CD3. In one aspect, the invention provides antibodies that specifically bind to CD3.

In one aspect, the invention provides an antibody that binds to CD3, wherein the antibody comprises a first antigen binding domain, comprising
(i) a heavy chain variable region (VH) comprising a heavy chain complementarity determining region (HCDR) 1 of SEQ ID NO: 1, a HCDR 2 of SEQ ID NO: 9, SEQ ID NO: 13 and SEQ ID NO: 15, and a HCDR 3 of SEQ ID NO: 3,
and
(ii) a light chain variable region (VL) comprising a light chain complementarity determining region (LCDR) 1 of SEQ ID NO: 5, a LCDR 2 of SEQ ID NO: 11 and a LCDR 3 of SEQ ID NO: 7.

In a one aspect, the invention provides an antibody that binds to CD3, wherein the antibody comprises a first antigen binding domain, comprising a heavy chain variable region 1, a HCDR 2 of SEQ ID NO: 9, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7.

In one aspect, the invention provides an antibody that binds to CD3, wherein the antibody comprises a first antigen binding domain, comprising a heavy chain variable region (VH) comprising a heavy chain complementarity determining region (HCDR) 1 of SEQ ID NO: 1, a HCDR 2 of SEQ ID NO: 13, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7.

In one aspect, the invention provides an antibody that binds to CD3, wherein the antibody comprises a first antigen binding domain, comprising a heavy chain variable region (VH) comprising a heavy chain complementarity determining region (HCDR) 1 of SEQ ID NO: 1, a HCDR 2 of SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7.

In one aspect, the antibody is a humanized antibody. In one aspect, the first antigen binding domain is (derived from) a humanized antibody. In one aspect, the first antigen binding domain is a humanized antigen binding domain (i.e. an antigen binding domain of a humanized antibody). In one aspect, the VH and/or the VL of the first antigen binding domain is a humanized variable region.

In one aspect, the VH and/or the VL of the first antigen binding domain comprises an acceptor human framework, e.g. a human immunoglobulin framework or a human consensus framework.

In one aspect, the VH of the first antigen binding domain comprises one or more heavy chain framework sequence (i.e. the FR1, FR2, FR3 and/or FR4 sequence) of a heavy chain variable region sequence selected from the group consisting of SEQ ID NO: 10, SEQ ID NO: 14, SEQ ID NO: 16, SEQ ID NO: 17, SEQ ID NO: 18 and SEQ ID NO: 19. In one aspect, the VH of the first antigen binding domain comprises an amino acid sequence that is at least about 95%, 96%, 97%, 98%, or 99% identical to an amino acid sequence selected from the group consisting of SEQ ID NO: 10, SEQ ID NO: 14, SEQ ID NO: 16, SEQ ID NO: 17, SEQ ID NO: 18 and SEQ ID NO: 19. In one aspect, the VH of the first antigen binding domain comprises an amino acid sequence that is at least about 95% identical to an amino acid sequence selected from the group consisting of SEQ ID NO: 10, SEQ ID NO: 14, SEQ ID NO: 16, SEQ ID NO: 17, SEQ ID NO: 18 and SEQ ID NO: 19. In one aspect, the VH of the first antigen binding domain comprises an amino acid sequence that is at least about 98% identical to an amino acid sequence selected from the group consisting of SEQ ID NO: 10, SEQ ID NO: 14, SEQ ID NO: 16, SEQ ID NO: 17, SEQ ID NO: 18 and SEQ ID NO: 19. In certain aspects, a VH sequence having at least 95%, 96%, 97%, 98%, or 99% identity contains substitutions (e.g., conservative substitutions), insertions, or deletions relative to the reference sequence, but an antibody comprising that sequence retains the ability to bind to CD3. In certain aspects, a total of 1 to 10 amino acids have been substituted, inserted and/or deleted in the amino acid sequence of SEQ ID NO: 10, SEQ ID NO: 14, SEQ ID NO: 16, SEQ ID NO: 17, SEQ ID NO: 18 or SEQ ID NO: 19. In certain aspects, substitutions, insertions, or deletions occur in regions outside the CDRs (i.e., in the FRs). In one aspect, the VH of the first antigen binding domain comprises an amino acid sequence selected from the group consisting of SEQ ID NO: 10, SEQ ID NO: 14, SEQ ID NO: 16, SEQ ID NO: 17, SEQ ID NO: 18 and SEQ ID NO: 19. Optionally, the VH of the first antigen binding domain comprises an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, including post-translational modifications of that sequence.

In one aspect, the VL of the first antigen binding domain comprises one or more light chain framework sequence (i.e. the FR1, FR2, FR3 and/or FR4 sequence) of the light chain variable region sequence of SEQ ID NO: 12. In one aspect, the VL of the first antigen binding domain comprises an amino acid sequence that is at least about 95%, 96%, 97%, 98%, or 99% identical to the amino acid sequence of SEQ ID NO: 12. In one aspect, the VL of the first antigen binding domain comprises an amino acid sequence that is at least about 95% identical to the amino acid sequence of SEQ ID NO: 12. In one aspect, the VL of the first antigen binding domain comprises an amino acid sequence that is at least about 98% identical to the amino acid sequence of SEQ ID NO: 12. In certain aspects, a VL sequence having at least 95%, 96%, 97%, 98%, or 99% identity contains substitutions (e.g., conservative substitutions), insertions, or deletions relative to the reference sequence, but an antibody comprising that sequence retains the ability to bind to CD3. In certain aspects, a total of 1 to 10 amino acids have been substituted, inserted and/or deleted in the amino acid sequence of SEQ ID NO: 12. In certain aspects, substitutions, insertions, or deletions occur in regions outside the CDRs (i.e., in the FRs). In one aspect, the VL of the first antigen binding domain comprises the amino acid sequence of SEQ ID NO: 12. Optionally, the VL of the first antigen binding domain comprises the amino acid sequence of SEQ ID NO: 12, including post-translational modifications of that sequence.

In one aspect, the VH of the first antigen binding domain comprises an amino acid sequence that is at least about 95%, 96%, 97%, 98%, or 99% identical to an amino acid sequence selected from the group consisting of SEQ ID NO: 10, SEQ ID NO: 14 and SEQ ID NO: 16, and/or the VL of the first antigen binding domain comprises an amino acid sequence that is at least about 95%, 96%, 97%, 98%, or 99% identical to the amino acid sequence of SEQ ID NO: 12. In one aspect, the VH of the first antigen binding domain comprises an amino acid sequence selected from the group consisting of SEQ ID NO: 10, SEQ ID NO: 14 and SEQ ID NO: 16, and/or the VL of the first antigen binding domain comprises the amino acid sequence of SEQ ID NO: 12.

In one aspect, the VH of the first antigen binding domain comprises amino acid substitutions L108S and S113G (Kabat numbering). Without wishing to be bound by theory, these mutations are predicted to reduce immunogenicity of the binder if placed in a VH/VL crossover Fab molecule. Accordingly, in one aspect, the VH of the first antigen binding domain comprises an amino acid sequence that is at least about 95%, 96%, 97%, 98%, or 99% identical to an amino acid sequence selected from the group consisting of SEQ ID NO: 17, SEQ ID NO: 18 and SEQ ID NO: 19, and/or the VL of the first antigen binding domain comprises an amino acid sequence that is at least about 95%, 96%, 97%, 98%, or 99% identical to the amino acid sequence of SEQ ID NO: 12. In one aspect, the VH of the first antigen binding domain comprises an amino acid sequence selected from the group consisting of SEQ ID NO: 17, SEQ ID NO: 18 and SEQ ID NO: 19, and/or the VL sequence of the first antigen binding domain comprises the amino acid sequence of SEQ ID NO: 12.

In one aspect, the VH of the first antigen binding domain comprises an amino acid sequence that is at least about 95%, 96%, 97%, 98%, or 99% identical to the amino acid sequence of SEQ ID NO: 10 or SEQ ID NO: 17, and/or the VL of the first antigen binding domain comprises an amino acid sequence that is at least about 95%, 96%, 97%, 98%, or 99% identical to the amino acid sequence of SEQ ID NO: 12. In one aspect, the VH of the first antigen binding domain comprises the amino acid sequence of SEQ ID NO: 10 or SEQ ID NO: 17, and/or the VL of the first antigen binding domain comprises the amino acid sequence of SEQ ID NO: 12.

In one aspect, the VH of the first antigen binding domain comprises an amino acid sequence that is at least about 95%, 96%, 97%, 98%, or 99% identical to the amino acid sequence of SEQ ID NO: 14 or SEQ ID NO: 18, and/or the VL of the first antigen binding domain comprises an amino acid sequence that is at least about 95%, 96%, 97%, 98%, or 99% identical to the amino acid sequence of SEQ ID NO: 12. In one aspect, the VH of the first antigen binding domain comprises the amino acid sequence of SEQ ID NO: 14 or SEQ ID NO: 18, and/or the VL of the first antigen binding domain comprises the amino acid sequence of SEQ ID NO: 12.

In one aspect, the VH of the first antigen binding domain comprises an amino acid sequence that is at least about 95%, 96%, 97%, 98%, or 99% identical to the amino acid sequence of SEQ ID NO: 16 or SEQ ID NO: 19, and/or the VL of the first antigen binding domain comprises an amino acid sequence that is at least about 95%, 96%, 97%, 98%, or 99% identical to the amino acid sequence of SEQ ID NO: 12. In one aspect, the VH of the first antigen binding domain comprises the amino acid sequence of SEQ ID NO: 16 or SEQ ID NO: 19, and/or the VL of the first antigen binding domain comprises the amino acid sequence of SEQ ID NO: 12.

In a further aspect, the invention provides an antibody that binds to CD3, wherein the antibody comprises a first antigen binding domain comprising a VH comprising an amino acid sequence selected from the group consisting of SEQ ID NO: 10, SEQ ID NO: 14 and SEQ ID NO: 16, and a VL comprising the amino acid sequence of SEQ ID NO: 12. In yet a further aspect, the invention provides an antibody that binds to CD3, wherein the antibody comprises a first antigen binding domain comprising a VH comprising an amino acid sequence selected from the group consisting of SEQ ID NO: 17, SEQ ID NO: 18 and SEQ ID NO: 19, and a VL comprising the amino acid sequence of SEQ ID NO: 12.

In one aspect, the invention provides an antibody that binds to CD3, wherein the antibody comprises a first antigen binding domain comprising a VH comprising the amino acid sequence of SEQ ID NO: 10 or SEQ ID NO: 17, and a VL comprising the amino acid sequence of SEQ ID NO: 12.

In one aspect, the invention provides an antibody that binds to CD3, wherein the antibody comprises a first antigen binding domain comprising a VH comprising the amino acid sequence of SEQ ID NO: 14 or SEQ ID NO: 18, and a VL comprising the amino acid sequence of SEQ ID NO: 12.

In one aspect, the invention provides an antibody that binds to CD3, wherein the antibody comprises a first antigen binding domain comprising a VH comprising the amino acid sequence of SEQ ID NO: 16 or SEQ ID NO: 19, and a VL comprising the amino acid sequence of SEQ ID NO: 12.

In a further aspect, the invention provides an antibody that binds to CD3, wherein the antibody comprises a first antigen binding domain comprising a VH sequence selected from the group consisting of SEQ ID NO: 10, SEQ ID NO: 14 and SEQ ID NO: 16, and a VL sequence of SEQ ID NO: 12. In yet a further aspect, the invention provides an antibody that binds to CD3, wherein the antibody comprises a first antigen binding domain comprising a VH sequence selected from the group consisting of SEQ ID NO: 17, SEQ ID NO: 18 and SEQ ID NO: 19, and a VL sequence of SEQ ID NO: 12.

In one aspect, the invention provides an antibody that binds to CD3, wherein the antibody comprises a first antigen binding domain comprising the VH sequence of SEQ ID NO: 10 or SEQ ID NO: 17, and a VL sequence of SEQ ID NO: 12.

In one aspect, the invention provides an antibody that binds to CD3, wherein the antibody comprises a first antigen binding domain comprising the VH sequence of SEQ ID NO: 14 or SEQ ID NO: 18, and a VL sequence of SEQ ID NO: 12.

In one aspect, the invention provides an antibody that binds to CD3, wherein the antibody comprises a first antigen binding domain comprising the VH sequence of SEQ ID NO: 16 or SEQ ID NO: 19, and a VL sequence of SEQ ID NO: 12.

In another aspect, the invention provides an antibody that binds to CD3, wherein the antibody comprises a first antigen binding domain comprising a VH comprising the heavy chain CDR sequences of a VH selected from the group consisting of SEQ ID NO: 10, SEQ ID NO: 14 and SEQ ID NO: 16, and a VL comprising the light chain CDR sequences of the VL of SEQ ID NO: 12.

In one aspect, the invention provides an antibody that binds to CD3, wherein the antibody comprises a first antigen binding domain comprising a VH comprising the heavy chain CDR sequences of the VH of SEQ ID NO: 10, and a VL comprising the light chain CDR sequences of the VL of SEQ ID NO: 12.

In one aspect, the invention provides an antibody that binds to CD3, wherein the antibody comprises a first antigen binding domain comprising a VH comprising the heavy chain CDR sequences of the VH of SEQ ID NO: 14, and a VL comprising the light chain CDR sequences of the VL of SEQ ID NO: 12.

In one aspect, the invention provides an antibody that binds to CD3, wherein the antibody comprises a first antigen binding domain comprising a VH comprising the heavy chain CDR sequences of the VH of SEQ ID NO: 16, and a VL comprising the light chain CDR sequences of the VL of SEQ ID NO: 12.

In a further aspect, the first antigen binding domain comprises the HCDR1, HCDR2 and HCDR3 amino acid sequences of a VH selected from the group consisting of SEQ ID NO: 10, SEQ ID NO: 14 and SEQ ID NO: 16, and the LCDR1, LCDR2 and LCDR3 amino acid sequences of the VL of SEQ ID NO: 12.

In one aspect, the first antigen binding domain comprises the HCDR1, HCDR2 and HCDR3 amino acid sequences of the VH of SEQ ID NO: 10, and the LCDR1, LCDR2 and LCDR3 amino acid sequences of the VL of SEQ ID NO: 12.

In one aspect, the first antigen binding domain comprises the HCDR1, HCDR2 and HCDR3 amino acid sequences of the VH of SEQ ID NO: 14, and the LCDR1, LCDR2 and LCDR3 amino acid sequences of the VL of SEQ ID NO: 12.

In one aspect, the first antigen binding domain comprises the HCDR1, HCDR2 and HCDR3 amino acid sequences of the VH of SEQ ID NO: 16, and the LCDR1, LCDR2 and LCDR3 amino acid sequences of the VL of SEQ ID NO: 12.

In one aspect, the VH of the first antigen binding domain comprises the heavy chain CDR sequences of a VH selected from the group consisting of SEQ ID NO: 10, SEQ ID NO: 14 and SEQ ID NO: 16, and a framework of at least 95%, 96%, 97%, 98% or 99% sequence identity to the framework sequence of a VH selected from the group consisting of SEQ ID NO: 10, SEQ ID NO: 14, SEQ ID NO: 16, SEQ ID NO: 17, SEQ ID NO: 18 and SEQ ID NO: 19. In one aspect, the VH of the first antigen binding domain comprises the heavy chain CDR sequences of a VH selected from the group consisting of SEQ ID NO: 10, SEQ ID NO: 14 and SEQ ID NO: 16, and a framework of at least 95% sequence identity to the framework sequence of a VH selected from the group consisting of SEQ ID NO: 10, SEQ ID NO: 14, SEQ ID NO: 16, SEQ ID NO: 17, SEQ ID NO: 18 and SEQ ID NO: 19. In another aspect, the VH of the first antigen binding domain comprises the heavy chain CDR sequences of a VH selected from the group consisting of SEQ ID NO: 10, SEQ ID NO: 14 and SEQ ID NO: 16, and a framework of at least 98% sequence identity to the framework sequence of a VH selected from the group consisting of SEQ ID NO: 10, SEQ ID NO: 14, SEQ ID NO: 16, SEQ ID NO: 17, SEQ ID NO: 18 and SEQ ID NO: 19.

In one aspect, the VH of the first antigen binding domain comprises the heavy chain CDR sequences of the VH of SEQ ID NO: 10, and a framework of at least 95%, 96%, 97%, 98% or 99% sequence identity to the framework sequence of the VH of SEQ ID NO: 10 or SEQ ID NO: 17. In one aspect, the VH of the first antigen binding domain comprises the heavy chain CDR sequences of the VH of SEQ ID NO: 10, and a framework of at least 95% sequence identity to the framework sequence of the VH of SEQ ID NO: 10 or SEQ ID NO: 17. In another aspect, the VH of the first antigen binding domain comprises the heavy chain CDR sequences of the VH of SEQ ID NO: 10, and a framework of at least 98% sequence identity to the framework sequence of the VH of SEQ ID NO: 10 or SEQ ID NO: 17.

In one aspect, the VH of the first antigen binding domain comprises the heavy chain CDR sequences of the VH of SEQ ID NO: 14, and a framework of at least 95%, 96%, 97%, 98% or 99% sequence identity to the framework sequence of the VH of SEQ ID NO: 14 or SEQ ID NO: 18. In one aspect, the VH of the first antigen binding domain comprises the heavy chain CDR sequences of the VH of SEQ ID NO: 14, and a framework of at least 95% sequence identity to the framework sequence of the VH of SEQ ID NO: 14 or SEQ ID NO: 18. In another aspect, the VH of the first antigen binding domain comprises the heavy chain CDR sequences of the VH of SEQ ID NO: 14, and a framework of at least 98% sequence identity to the framework sequence of the VH of SEQ ID NO: 14 or SEQ ID NO: 18.

In one aspect, the VH of the first antigen binding domain comprises the heavy chain CDR sequences of the VH of SEQ ID NO: 16, and a framework of at least 95%, 96%, 97%, 98% or 99% sequence identity to the framework sequence of the VH of SEQ ID NO: 16 or SEQ ID NO: 19. In one aspect, the VH of the first antigen binding domain comprises the heavy chain CDR sequences of the VH of SEQ ID NO: 16, and a framework of at least 95% sequence identity to the framework sequence of the VH of SEQ ID NO: 16 or SEQ ID NO: 19. In another aspect, the VH of the first antigen binding domain comprises the heavy chain CDR sequences of the VH of SEQ ID NO: 16, and a framework of at least 98% sequence identity to the framework sequence of the VH of SEQ ID NO: 16 or SEQ ID NO: 19.

In one aspect, the VL of the first antigen binding domain comprises the light chain CDR sequences of the VL of SEQ ID NO: 12 and a framework of at least 95%, 96%, 97%, 98% or 99% sequence identity to the framework sequence of the VL of SEQ ID NO: 12. In one aspect, the VL of the first antigen binding domain comprises the light chain CDR sequences of the VL of SEQ ID NO: 12 and a framework of at least 95% sequence identity to the framework sequence of the VL of SEQ ID NO: 12. In another aspect, the VL of the first antigen binding domain comprises the light chain CDR sequences of the VL of SEQ ID NO: 12 and a framework of at least 98% sequence identity to the framework sequence of the VL of SEQ ID NO: 12.

In one aspect, the invention provides an antibody that binds to CD3, wherein the antibody comprises a first antigen binding domain comprising a VH sequence as in any of the aspects provided above, and a VL sequence as in any of the aspects provided above.

In one aspect, the antibody comprises a human constant region. In one aspect, the antibody is an immunoglobulin molecule comprising a human constant region, particularly an IgG class immunoglobulin molecule comprising a human CH1, CH2, CH3 and/or CL domain. Exemplary sequences of human constant domains are given in SEQ ID NOs 39 and 40 (human kappa and lambda CL domains, respectively) and SEQ ID NO: 41 (human IgG₁ heavy chain constant domains CH1-CH2-CH3). In one aspect, the antibody comprises a light chain constant region comprising an amino acid sequence that is at least about 95%, 96%, 97%, 98%, 99% or 100% identical to the amino acid sequence of SEQ ID NO: 39 or SEQ ID NO: 40, particularly the amino acid sequence of SEQ ID NO: 39. In one aspect, the antibody comprises a heavy chain constant region comprising an amino acid sequence that is at least about 95%, 96%, 97%, 98%, 99% or 100% identical to the amino acid sequence of SEQ ID NO: 41. Particularly, the heavy chain constant region may comprise amino acid mutations in the Fc domain as described herein.

In one aspect, the first antigen binding domain comprises a human constant region. In one aspect, the first antigen binding domain is a Fab molecule comprising a human constant region, particularly a human CH1 and/or CL domain. In one aspect, the first antigen binding domain comprises a light chain constant region comprising an amino acid sequence that is at least about 95%, 96%, 97%, 98%, 99% or 100% identical to the amino acid sequence of SEQ ID NO: 39 or SEQ ID NO: 40, particularly the amino acid sequence of SEQ ID NO: 39. Particularly, the light chain constant region may comprise amino acid mutations as described herein under “charge modifications” and/or may comprise deletion or substitutions of one or more (particularly two) N-terminal amino acids if in a crossover Fab molecule. In some aspects, the first antigen binding domain comprises a heavy chain constant region comprising an amino acid sequence that is at least about 95%, 96%, 97%, 98%, 99% or 100% identical to the CH1 domain sequence comprised in the amino acid sequence of SEQ ID NO: 41. Particularly, the heavy chain constant region (specifically CH1 domain) may comprise amino acid mutations as described herein under “charge modifications”.

In one aspect, the antibody is a monoclonal antibody.

In one aspect, the antibody is an IgG, particularly an IgG₁, antibody. In one aspect, the antibody is a full-length antibody.

In another aspect, the antibody is an antibody fragment selected from the group of an Fv molecule, a scFv molecule, a Fab molecule, and a F(ab′)₂ molecule; particularly a Fab molecule. In another aspect, the antibody fragment is a diabody, a triabody or a tetrabody.

In one aspect, the first antigen binding domain is a Fab molecule. In a preferred aspect the first antigen binding domain is a Fab molecule wherein the variable domains VL and VH or the constant domains CL and CH1, particularly the variable domains VL and VH, of the Fab light chain and the Fab heavy chain are replaced by each other (i.e. the first antigen binding domain is a crossover Fab molecule).

In a further aspect, the antibody according to any of the above aspects may incorporate any of the features, singly or in combination, as described in sections II. A. 1.-8. below.

In a preferred aspect, the antibody comprises an Fc domain, particularly an IgG Fc domain, more particularly an IgG1 Fc domain. In one aspect the Fc domain is a human Fc domain. In one aspect, the Fc domain is a human IgG₁ Fc domain. The Fc domain is composed of a first and a second subunit and may incorporate any of the features, singly or in combination, described hereinbelow in relation to Fc domain variants (section II. A. 8).

In another preferred aspect, the antibody comprises a second and optionally a third antigen binding domain that binds to a second antigen (i.e. the antibody is a multispecific antibody, as further described hereinbelow (section II. A. 7).

### 1. Antibody Fragments

In certain aspects, an antibody provided herein is an antibody fragment.

In one aspect, the antibody fragment is a Fab, Fab′, Fab′-SH, or F(ab′)₂ molecule, in particular a Fab molecule as described herein. “Fab′ molecule” differ from Fab molecules by the addition of residues at the carboxy terminus of the CH1 domain including one or more cysteines from the antibody hinge region. Fab′-SH are Fab′ molecules in which the cysteine residue(s) of the constant domains bear a free thiol group. Pepsin treatment yields an F(ab′)₂ molecule that has two antigen-binding sites (two Fab molecules) and a part of the Fc region.

In another aspect, the antibody fragment is a diabody, a triabody or a tetrabody. “Diabodies” are antibody fragments with two antigen-binding sites that may be bivalent or bispecific. Sec, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab molecule. A “single chain Fab molecule” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab molecules are stabilized via the natural disulfide bond between the CL domain and the CH1 domain. In addition, these single chain Fab molecules might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 2. Humanized Antibodies

In certain aspects, an antibody provided herein is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 3. Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e.g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### 4. Cysteine Engineered Antibody Variants

In certain aspects, it may be desirable to create cysteine engineered antibodies, e.g., THIOMAB™ antibodies, in which one or more residues of an antibody are substituted with cysteine residues. In preferred aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### 5. Antibody Derivatives

In certain aspects, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 6. Immunoconjugates

The invention also provides immunoconjugates comprising an anti-CD3 antibody herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody of the invention conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody of the invention conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc^{99m} or I¹²³, or a spin label for nuclear magnetic resonance (NMR) imaging (also known as magnetic resonance imaging, MRI), such as I¹²³, I¹³¹, In¹¹¹, F¹⁹, C¹³, N¹⁵, O¹⁷, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### 7. Multispecific Antibodies

In certain aspects, an antibody provided herein is a multispecific antibody, particularly a bispecific antibody. Multispecific antibodies are monoclonal antibodies that have binding specificities for at least two different antigenic determinants (e.g., two different proteins, or two different epitopes on the same protein). In certain aspects, the multispecific antibody has three or more binding specificities. In certain aspects, one of the binding specificities is for CD3 and the other specificity is for any other antigen. In certain aspects, multispecific antibodies may bind to two (or more) different epitopes of CD3. Multispecific (e.g., bispecific) antibodies may also be used to localize cytotoxic agents or cells to cells which express CD3. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229:81 (1985)); using leucine zippers to produce bi-specific antibodies (see, e.g., Kostelny et al., J. Immunol., 148 (5): 1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The multispecific antibody or antigen binding fragment thereof also includes a “Dual Acting FAb” or “DAF” comprising an antigen binding site that binds to CD3 as well as another different antigen, or two different epitopes of CD3 (see, e.g., US 2008/0069820 and WO 2015/095539).

Multi-specific antibodies may also be provided in an asymmetric form with a domain crossover in one or more binding arms of the same antigen specificity (so-called “CrossMab” technology), i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in preferred aspects, an antibody provided herein is a multispecific antibody, particularly a bispecific antibody, wherein one of the binding specificities is for CD3 and the other is for target cell antigen.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5 (8) (2016) e1203498.

Preferred aspects of the multispecific antibodies of the present invention are described in the following.

In one aspect, the invention provides an antibody that binds to CD3, comprising a first antigen binding domain that binds to CD3, as described herein, and comprising a second and optionally a third antigen binding domain that binds to a second antigen.

According to preferred aspects of the invention, the antigen binding domains comprised in the antibody are Fab molecules (i.e. antigen binding domains composed of a heavy and a light chain, each comprising a variable and a constant domain). In one aspect, the first, the second and/or, where present, the third antigen binding domain is a Fab molecule. In one aspect, said Fab molecule is human. In a preferred aspect, said Fab molecule is humanized. In another preferred aspect, said Fab molecule comprises human heavy and light chain constant domains.

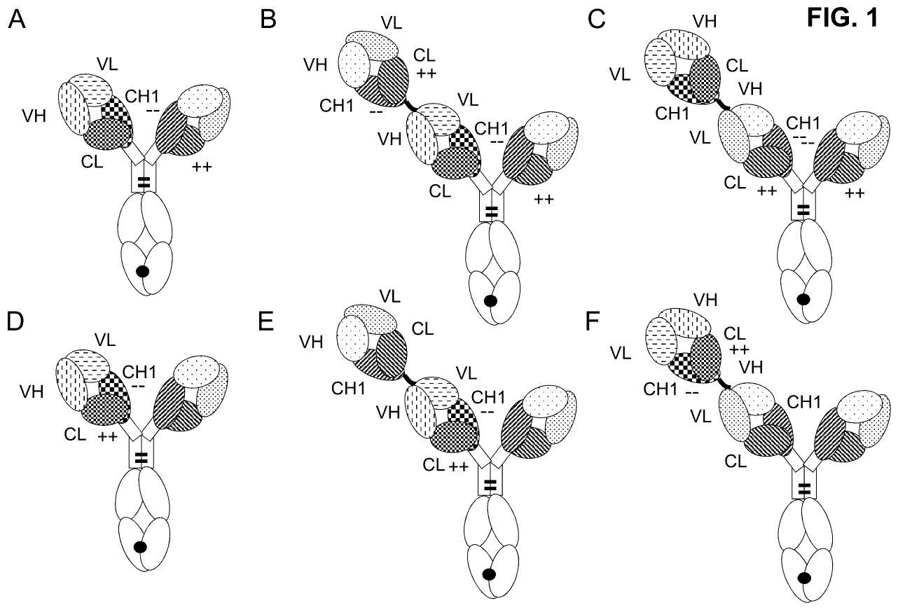

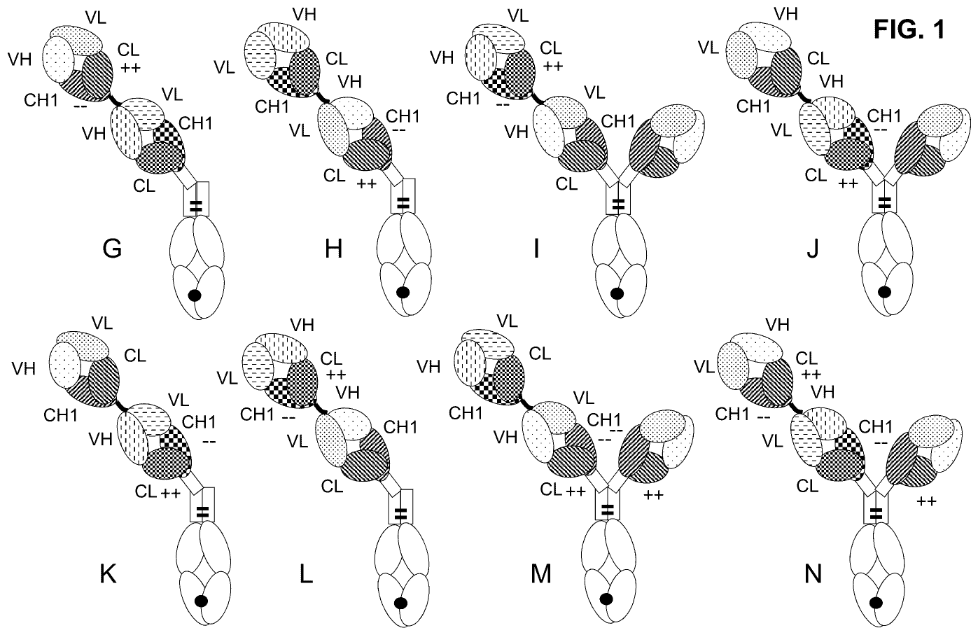

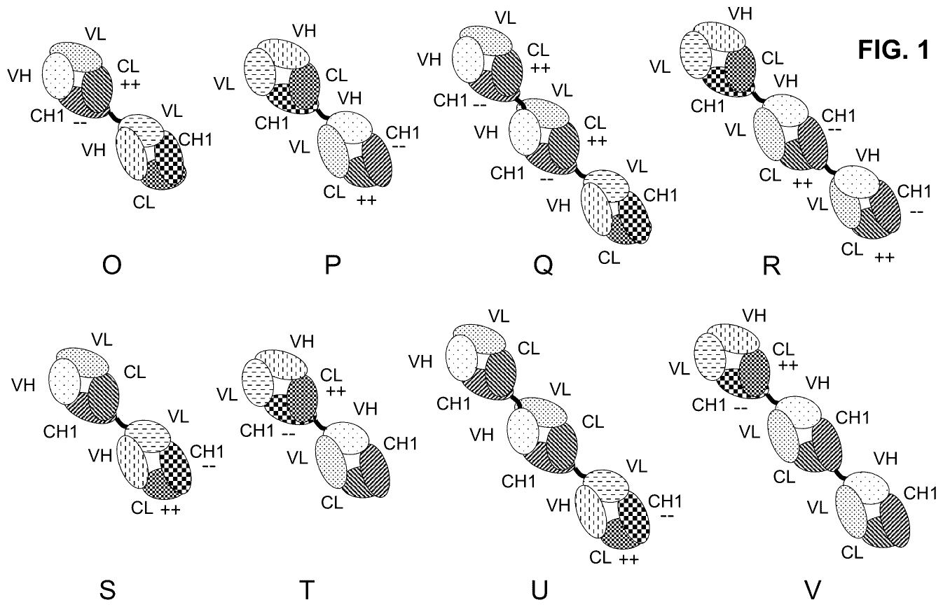

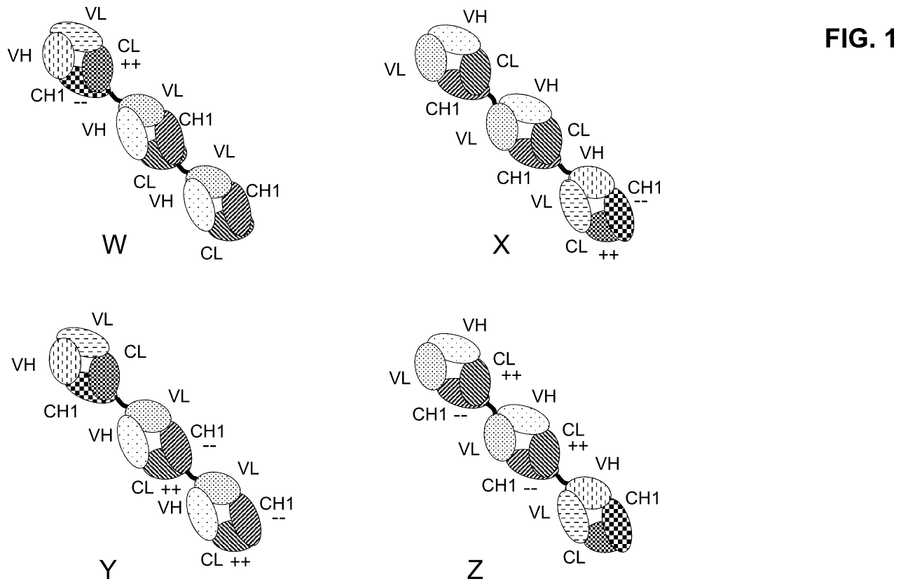

Preferably, at least one of the antigen binding domains is a crossover Fab molecule. Such modification reduces mispairing of heavy and light chains from different Fab molecules, thereby improving the yield and purity of the (multispecific) antibody of the invention in recombinant production. In a preferred crossover Fab molecule useful for the (multispecific) antibody of the invention, the variable domains of the Fab light chain and the Fab heavy chain (VL and VH, respectively) are exchanged. Even with this domain exchange, however, the preparation of the (multispecific) antibody may comprise certain side products due to a so-called Bence Jones-type interaction between mispaired heavy and light chains (see Schaefer et al, PNAS, 108 (2011) 11187-11191). To further reduce mispairing of heavy and light chains from different Fab molecules and thus increase the purity and yield of the desired (multispecific) antibody, charged amino acids with opposite charges may be introduced at specific amino acid positions in the CH1 and CL domains of either the Fab molecule binding to the first antigen (CD3), or the Fab molecule(s) binding to the second antigen (e.g. a target cell antigen such as HER2), as further described herein. Charge modifications are made either in the conventional Fab molecule(s) comprised in the (multispecific) antibody (such as shown e.g. in FIGS. 1A-1C, FIGS. 1G-1J), or in the VH/VL crossover Fab molecule(s) comprised in the (multispecific) antibody (such as shown e.g. in FIGS. 1D-IF, FIGS. 1K-N) (but not in both). In preferred aspects, the charge modifications are made in the conventional Fab molecule(s) comprised in the (multispecific) antibody (which in preferred aspects bind(s) to the second antigen, e.g. a target cell antigen such as HER2).

In a preferred aspect according to the invention, the (multispecific) antibody is capable of simultaneous binding to the first antigen (i.e. CD3), and the second antigen (e.g. a target cell antigen such as HER2). In one aspect, the (multispecific) antibody is capable of crosslinking a T cell and a target cell by simultaneous binding to CD3 and a target cell antigen. In an even more preferred aspect, such simultaneous binding results in lysis of the target cell, particularly a target cell antigen (e.g. HER2)-expressing tumor cell. In one aspect, such simultaneous binding results in activation of the T cell. In other aspects, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one aspect, binding of the (multispecific) antibody to CD3 without simultaneous binding to the target cell antigen does not result in T cell activation.

In one aspect, the (multispecific) antibody is capable of re-directing cytotoxic activity of a T cell to a target cell. In a preferred aspect, said re-direction is independent of MHC-mediated peptide antigen presentation by the target cell and and/or specificity of the T cell.

Preferably, a T cell according to any of the aspects of the invention is a cytotoxic T cell. In some aspects the T cell is a CD4⁺ or a CD8⁺ T cell, particularly a CD8⁺ T cell.

### a) First Antigen Binding Domain

The (multispecific) antibody of the invention comprises at least one antigen binding domain (the first antigen binding domain) that binds to CD3. In preferred aspects, CD3 is human CD3 (SEQ ID NO: 36) or cynomolgus CD3 (SEQ ID NO: 37) most particularly human CD3. In one aspect the first antigen binding domain is cross-reactive for (i.e. specifically binds to) human and cynomolgus CD3. In some aspects, CD3 is the epsilon subunit of CD3 (CD3 epsilon).

In a preferred aspect, the (multispecific) antibody comprises not more than one antigen binding domain that binds to CD3. In one aspect the (multispecific) antibody provides monovalent binding to CD3.

In one aspect, the antigen binding domain that binds to CD3 is an antibody fragment selected from the group of an Fv molecule, a scFv molecule, a Fab molecule, and a F(ab′)₂ molecule. In a preferred aspect, the antigen binding domain that binds to CD3 is a Fab molecule.

In preferred aspects, the antigen binding domain that binds to CD3 is a crossover Fab molecule as described herein, i.e. a Fab molecule wherein the variable domains VH and VL or the constant domains CH1 and CL of the Fab heavy and light chains are exchanged/replaced by each other. In such aspects, the antigen binding domain(s) that binds to the second antigen (e.g. a target cell antigen such as HER2) is preferably a conventional Fab molecule. In aspects where there is more than one antigen binding domain, particularly Fab molecule, that binds to a second antigen comprised in the (multispecific) antibody, the antigen binding domain that binds to CD3 preferably is a crossover Fab molecule and the antigen binding domain that bind to the second antigen are conventional Fab molecules.

In alternative aspects, the antigen binding domain that binds to CD3 is a conventional Fab molecule. In such aspects, the antigen binding domain(s) that binds to the second antigen (e.g. a target cell antigen such as HER2) is a crossover Fab molecule as described herein, i.e. a Fab molecule wherein the variable domains VH and VL or the constant domains CH1 and CL of the Fab heavy and light chains are exchanged/replaced by each other. In aspects where there is more than one antigen binding domain, particularly Fab molecule, that binds to CD3 comprised in the (multispecific) antibody, the antigen binding domain that binds to the second antigen preferably is a crossover Fab molecule and the antigen binding domains that bind to CD3 are conventional Fab molecules.

In preferred aspects, the first antigen binding domain is a Fab molecule wherein the variable domains VL and VH or the constant domains CL and CH1, particularly the variable domains VL and VH, of the Fab light chain and the Fab heavy chain are replaced by each other (i.e. according to such aspect, the first antigen binding domain is a crossover Fab molecule wherein the variable or constant domains of the Fab light chain and the Fab heavy chain are exchanged). In one such aspect, the second (and the third, if any) antigen binding domain is a conventional Fab molecule.

In one aspect, not more than one antigen binding domain that binds to CD3 is present in the (multispecific) antibody (i.e. the antibody provides monovalent binding to CD3).

### b) Second (and Third) Antigen Binding Domain

In certain aspects, the (multispecific) antibody of the invention comprises at least one antigen binding domain, particularly a Fab molecule, that binds to a second antigen. The second antigen preferably is not CD3, i.e. different from CD3. In one aspect, the second antigen is an antigen expressed on a different cell than CD3 (e.g. expressed on a cell other than a T cell). In one aspect, the second antigen is a target cell antigen, particularly a tumor cell antigen. In a specific aspect, the second antigen is HER2. The second antigen binding domain is able to direct the (multispecific) antibody to a target site, for example to a specific type of tumor cell that expresses the second antigen.

In one aspect, the antigen binding domain that binds to the second antigen is an antibody fragment selected from the group of an Fv molecule, a scFv molecule, a Fab molecule, and a F(ab′)₂ molecule. In a preferred aspect, the antigen binding domain that binds to the second antigen is a Fab molecule.

In certain aspects, the (multispecific) antibody comprises two antigen binding domains, particularly Fab molecules, that bind to the second antigen. In a preferred such aspect, each of these antigen binding domains binds to the same antigenic determinant. In an even more preferred aspect, all of these antigen binding domains are identical, i.e. they have the same molecular format (e.g. conventional or crossover Fab molecule) and comprise the same amino acid sequences including the same amino acid substitutions in the CH1 and CL domain as described herein (if any). In one aspect, the (multispecific) antibody comprises not more than two antigen binding domains, particularly Fab molecules, that bind to the second antigen.

In preferred aspects, the antigen binding domain(s) that bind to the second antigen is/are a conventional Fab molecule. In such aspects, the antigen binding domain(s) that binds to CD3 is a crossover Fab molecule as described herein, i.e. a Fab molecule wherein the variable domains VH and VL or the constant domains CH1 and CL of the Fab heavy and light chains are exchanged/replaced by each other.

In alternative aspects, the antigen binding domain(s) that bind to the second antigen is/are a crossover Fab molecule as described herein, i.e. a Fab molecule wherein the variable domains VH and VL or the constant domains CH1 and CL of the Fab heavy and light chains are exchanged/replaced by each other. In such aspects, the antigen binding domain(s) that binds to CD3 is a conventional Fab molecule.

In one aspect, the second (and, where present, the third) antigen binding domain comprises a human constant region. In one aspect, the second (and, where present, the third) antigen binding domain is a Fab molecule comprising a human constant region, particularly a human CH1 and/or CL domain. Exemplary sequences of human constant domains are given in SEQ ID NOs 39 and 40 (human kappa and lambda CL domains, respectively) and SEQ ID NO: 41 (human IgG1 heavy chain constant domains CH1-CH2-CH3). In one aspect, the second (and, where present, the third) antigen binding domain comprises a light chain constant region comprising an amino acid sequence that is at least about 95%, 96%, 97%, 98%, 99% or 100% identical to the amino acid sequence of SEQ ID NO: 39 or SEQ ID NO: 40, particularly the amino acid sequence of SEQ ID NO: 39. Particularly, the light chain constant region may comprise amino acid mutations as described herein under “charge modifications” and/or may comprise deletion or substitutions of one or more (particularly two) N-terminal amino acids if in a crossover Fab molecule. In some aspects, the second (and, where present, the third) antigen binding domain comprises a heavy chain constant region comprising an amino acid sequence that is at least about 95%, 96%, 97%, 98%, 99% or 100% identical to the CH1 domain sequence comprised in the amino acid sequence of SEQ ID NO: 41. Particularly, the heavy chain constant region (specifically CH1 domain) may comprise amino acid mutations as described herein under “charge modifications”.

In some aspects, the second antigen is HER2, particularly human HER2.

In one aspect, the second (and, where present, the third) antigen binding domain comprises a heavy chain variable region (VH) comprising a heavy chain complementarity determining region (HCDR) 1 of SEQ ID NO: 20, a HCDR 2 of SEQ ID NO: 21, and a HCDR 3 of SEQ ID NO: 22, and a light chain variable region (VL) comprising a light chain complementarity determining region (LCDR) 1 of SEQ ID NO: 24, a LCDR 2 of SEQ ID NO: 25 and a LCDR 3 of SEQ ID NO: 26.

In one aspect, the second (and, where present, the third) antigen binding domain is (derived from) a humanized antibody. In one aspect, the second (and, where present, the third) antigen binding domain is a humanized antigen binding domain (i.e. an antigen binding domain of a humanized antibody). In one aspect, the VH and/or the VL of the second (and, where present, the third) antigen binding domain is a humanized variable region.

In one aspect, the VH and/or the VL of the second (and, where present, the third) antigen binding domain comprises an acceptor human framework, e.g. a human immunoglobulin framework or a human consensus framework.

In one aspect, the VH of the second (and, where present, the third) antigen binding domain comprises one or more heavy chain framework sequence (i.e. the FR1, FR2, FR3 and/or FR4 sequence) of the heavy chain variable region sequence of SEQ ID NO: 23. In one aspect, the VH comprises an amino acid sequence that is at least about 95%, 96%, 97%, 98%, or 99% identical to the amino acid sequence of SEQ ID NO: 23. In one aspect, the VH comprises an amino acid sequence that is at least about 95% identical to the amino acid sequence of SEQ ID NO: 23. In one aspect, the VH comprises an amino acid sequence that is at least about 98% identical to the amino acid sequence of SEQ ID NO: 23. In certain aspects, a VH sequence having at least 95%, 96%, 97%, 98%, or 99% identity contains substitutions (e.g., conservative substitutions), insertions, or deletions relative to the reference sequence, but an antibody comprising that sequence retains the ability to bind to HER2. In certain aspects, a total of 1 to 10 amino acids have been substituted, inserted and/or deleted in the amino acid sequence of SEQ ID NO: 23. In certain aspects, substitutions, insertions, or deletions occur in regions outside the CDRs (i.e., in the FRs). In one aspect, the VH comprises the amino acid sequence of SEQ ID NO: 23. Optionally, the VH comprises the amino acid sequence of SEQ ID NO: 23, including post-translational modifications of that sequence.

In one aspect, the VL of the second (and, where present, the third) antigen binding domain comprises one or more light chain framework sequence (i.e. the FR1, FR2, FR3 and/or FR4 sequence) of the light chain variable region sequence of SEQ ID NO: 27. In one aspect, the VL comprises an amino acid sequence that is at least about 95%, 96%, 97%, 98%, or 99% identical to the amino acid sequence of SEQ ID NO: 27. In one aspect, the VL comprises an amino acid sequence that is at least about 95% identical to the amino acid sequence of SEQ ID NO: 27. In one aspect, the VL comprises an amino acid sequence that is at least about 98% identical to the amino acid sequence of SEQ ID NO: 27. In certain aspects, a VL sequence having at least 95%, 96%, 97%, 98%, or 99% identity contains substitutions (e.g., conservative substitutions), insertions, or deletions relative to the reference sequence, but an antibody comprising that sequence retains the ability to bind to HER2. In certain aspects, a total of 1 to 10 amino acids have been substituted, inserted and/or deleted in the amino acid sequence of SEQ ID NO: 27. In certain aspects, substitutions, insertions, or deletions occur in regions outside the CDRs (i.e., in the FRs). In one aspect, the VL comprises the amino acid sequence of SEQ ID NO: 27. Optionally, the VL comprises the amino acid sequence of SEQ ID NO: 27, including post-translational modifications of that sequence.

In one aspect, the VH of the second (and, where present, the third) antigen binding domain comprises an amino acid sequence that is at least about 95%, 96%, 97%, 98%, or 99% identical to the amino acid sequence of SEQ ID NO: 23, and the VL of the second (and, where present, the third) antigen binding domain comprises an amino acid sequence that is at least about 95%, 96%, 97%, 98%, or 99% identical to the amino acid sequence of SEQ ID NO: 27. In one aspect, the VH comprises the amino acid sequence of SEQ ID NO: 23 and the VL comprises the amino acid sequence of SEQ ID NO: 27.

In a further aspect, the second (and, where present, the third) antigen binding domain comprises a VH comprising the amino acid sequence of SEQ ID NO: 23 and a VL comprising the amino acid sequence of SEQ ID NO: 27.

In a further aspect, the second (and, where present, the third) antigen binding domain comprises a VH sequence of SEQ ID NO: 23 and a VL sequence of SEQ ID NO: 27.

In another aspect, the second (and, where present, the third) antigen binding domain comprises a VH comprising the heavy chain CDR sequences of the VH of SEQ ID NO: 23, and a VL comprising the light chain CDR sequences of the VL of SEQ ID NO: 27.

In a further aspect, the second (and, where present, the third) antigen binding domain comprises the HCDR1, HCDR2 and HCDR3 amino acid sequences of the VH of SEQ ID NO: 23 and the LCDR1, LCDR2 and LCDR3 amino acid sequences of the VL of SEQ ID NO: 27.

In one aspect, the VH of the second (and, where present, the third) antigen binding domain comprises the heavy chain CDR sequences of the VH of SEQ ID NO: 23 and a framework of at least 95%, 96%, 97%, 98% or 99% sequence identity to the framework sequence of the VH of SEQ ID NO: 23. In one aspect, the VH comprises the heavy chain CDR sequences of the VH of SEQ ID NO: 23 and a framework of at least 95% sequence identity to the framework sequence of the VH of SEQ ID NO: 23. In another aspect, the VH comprises the heavy chain CDR sequences of the VH of SEQ ID NO: 23 and a framework of at least 98% sequence identity to the framework sequence of the VH of SEQ ID NO: 23.

In one aspect, the VL of the second (and, where present, the third) antigen binding domain comprises the light chain CDR sequences of the VL of SEQ ID NO: 27 and a framework of at least 95%, 96%, 97%, 98% or 99% sequence identity to the framework sequence of the VL of SEQ ID NO: 27. In one aspect, the VL comprises the light chain CDR sequences of the VL of SEQ ID NO: 27 and a framework of at least 95% sequence identity to the framework sequence of the VL of SEQ ID NO: 27. In another aspect, the VL comprises the light chain CDR sequences of the VL of SEQ ID NO: 27 and a framework of at least 98% sequence identity to the framework sequence of the VL of SEQ ID NO: 27.

### c) Charge Modifications

The (multispecific) antibody of the invention may comprise amino acid substitutions in Fab molecules comprised therein which are particularly efficient in reducing mispairing of light chains with non-matching heavy chains (Bence-Jones-type side products), which can occur in the production of Fab-based multispecific antibodies with a VH/VL exchange in one (or more, in case of molecules comprising more than two antigen-binding Fab molecules) of their binding arms (see also PCT publication no. WO 2015/150447, particularly the examples therein, incorporated herein by reference in its entirety). The ratio of a desired (multispecific) antibody compared to undesired side products, in particular Bence Jones-type side products occurring in multispecific antibodies with a VH/VL domain exchange in one of their binding arms, can be improved by the introduction of charged amino acids with opposite charges at specific amino acid positions in the CH1 and CL domains (sometimes referred to herein as “charge modifications”).

Accordingly, in some aspects wherein the first and the second (and, where present, the third) antigen binding domain of the (multispecific) antibody are both Fab molecules, and in one of the antigen binding domains (particularly the first antigen binding domain) the variable domains VL and VH of the Fab light chain and the Fab heavy chain are replaced by each other,
i) in the constant domain CL of the second (and, where present, the third) antigen binding domain the amino acid at position 124 is substituted by a positively charged amino acid (numbering according to Kabat), and wherein in the constant domain CH1 of the second (and, where present, the third) antigen binding domain the amino acid at position 147 or the amino acid at position 213 is substituted by a negatively charged amino acid (numbering according to Kabat EU index); or
ii) in the constant domain CL of the first antigen binding domain the amino acid at position 124 is substituted by a positively charged amino acid (numbering according to Kabat), and wherein in the constant domain CH1 of the first antigen binding domain the amino acid at position 147 or the amino acid at position 213 is substituted by a negatively charged amino acid (numbering according to Kabat EU index).

The (multispecific) antibody does not comprise both modifications mentioned under i) and ii). The constant domains CL and CH1 of the antigen binding domain having the VH/VL exchange are not replaced by each other (i.e. remain unexchanged).

In a more specific aspect,
i) in the constant domain CL of the second (and, where present, the third) antigen binding domain the amino acid at position 124 is substituted independently by lysine (K), arginine (R) or histidine (H) (numbering according to Kabat), and in the constant domain CH1 of the second (and, where present, the third) antigen binding domain the amino acid at position 147 or the amino acid at position 213 is substituted independently by glutamic acid (E), or aspartic acid (D) (numbering according to Kabat EU index); or
ii) in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 or the amino acid at position 213 is substituted independently by glutamic acid (E), or aspartic acid (D) (numbering according to Kabat EU index).

In one such aspect, in the constant domain CL of the second (and, where present, the third) antigen binding domain the amino acid at position 124 is substituted independently by lysine (K), arginine (R) or histidine (H) (numbering according to Kabat), and in the constant domain CH1 of the second (and, where present, the third) antigen binding domain the amino acid at position 147 or the amino acid at position 213 is substituted independently by glutamic acid (E), or aspartic acid (D) (numbering according to Kabat EU index).

In a further aspect, in the constant domain CL of the second (and, where present, the third) antigen binding domain the amino acid at position 124 is substituted independently by lysine (K), arginine (R) or histidine (H) (numbering according to Kabat), and in the constant domain CH1 of the second (and, where present, the third) antigen binding domain the amino acid at position 147 is substituted independently by glutamic acid (E), or aspartic acid (D) (numbering according to Kabat EU index).

In a preferred aspect, in the constant domain CL of the second (and, where present, the third) antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second (and, where present, the third) antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more preferred aspect, in the constant domain CL of the second (and, where present, the third) antigen binding domain the amino acid at position 124 is substituted by lysine (K) (numbering according to Kabat) and the amino acid at position 123 is substituted by lysine (K) (numbering according to Kabat), and in the constant domain CH1 of the second (and, where present, the third) antigen binding domain the amino acid at position 147 is substituted by glutamic acid (E) (numbering according to Kabat EU index) and the amino acid at position 213 is substituted by glutamic acid (E) (numbering according to Kabat EU index).

In an even more preferred aspect, in the constant domain CL of the second (and, where present, the third) antigen binding domain the amino acid at position 124 is substituted by lysine (K) (numbering according to Kabat) and the amino acid at position 123 is substituted by arginine (R) (numbering according to Kabat), and in the constant domain CH1 of the second (and, where present, the third) antigen binding domain the amino acid at position 147 is substituted by glutamic acid (E) (numbering according to Kabat EU index) and the amino acid at position 213 is substituted by glutamic acid (E) (numbering according to Kabat EU index).

In preferred aspects, if amino acid substitutions according to the above aspects are made in the constant domain CL and the constant domain CH1 of the second (and, where present, the third) antigen binding domain, the constant domain CL of the second (and, where present, the third) antigen binding domain is of kappa isotype.

Alternatively, the amino acid substitutions according to the above aspects may be made in the constant domain CL and the constant domain CH1 of the first antigen binding domain instead of in the constant domain CL and the constant domain CH1 of the second (and, where present, the third) antigen binding domain. In preferred such aspects, the constant domain CL of the first antigen binding domain is of kappa isotype.

Accordingly, in one aspect, in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 or the amino acid at position 213 is substituted independently by glutamic acid (E), or aspartic acid (D) (numbering according to Kabat EU index).

In a further aspect, in the constant domain CL of the first antigen binding domain the amino acid at position 124 is substituted independently by lysine (K), arginine (R) or histidine (H) (numbering according to Kabat), and in the constant domain CH1 of the first antigen binding domain the amino acid at position 147 is substituted independently by glutamic acid (E), or aspartic acid (D) (numbering according to Kabat EU index).

In still another aspect, in the constant domain CL of the first antigen binding domain the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the first antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the constant domain CL of the first antigen binding domain the amino acid at position 124 is substituted by lysine (K) (numbering according to Kabat) and the amino acid at position 123 is substituted by lysine (K) (numbering according to Kabat), and in the constant domain CH1 of the first antigen binding domain the amino acid at position 147 is substituted by glutamic acid (E) (numbering according to Kabat EU index) and the amino acid at position 213 is substituted by glutamic acid (E) (numbering according to Kabat EU index).

In another aspect, in the constant domain CL of the first antigen binding domain the amino acid at position 124 is substituted by lysine (K) (numbering according to Kabat) and the amino acid at position 123 is substituted by arginine (R) (numbering according to Kabat), and in the constant domain CH1 of the first antigen binding domain the amino acid at position 147 is substituted by glutamic acid (E) (numbering according to Kabat EU index) and the amino acid at position 213 is substituted by glutamic acid (E) (numbering according to Kabat EU index).

In a preferred aspect, the (multispecific) antibody of the invention comprises
(A) a first antigen binding domain that binds to CD3, wherein the first antigen binding domain is a Fab molecule wherein the variable domains VL and VH of the Fab light chain and the Fab heavy chain are replaced by each other, and comprises (i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7 and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12, and
(B) a second and optionally a third antigen binding domain that binds to a second antigen;
wherein in the constant domain CL of the second (and, where present, the third) antigen binding domain the amino acid at position 124 is substituted independently by lysine (K), arginine (R) or histidine (H) (numbering according to Kabat) (in a preferred aspect independently by lysine (K) or arginine (R)) and the amino acid at position 123 is substituted independently by lysine (K), arginine (R) or histidine (H) (numbering according to Kabat) (in a preferred aspect independently by lysine (K) or arginine (R)), and in the constant domain CH1 of the second (and, where present, the third) antigen binding domain the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

### d) Multispecific Antibody Formats

The (multispecific) antibody according to the present invention can have a variety of configurations. Exemplary configurations are depicted in FIGS. 1A-1Z.

In preferred aspects, the antigen binding domains comprised in the (multispecific) antibody are Fab molecules. In such aspects, the first, second, third etc. antigen binding domain may be referred to herein as first, second, third etc. Fab molecule, respectively.

In one aspect, the first and the second antigen binding domain of the (multispecific) antibody are fused to each other, optionally via a peptide linker. In preferred aspects, the first and the second antigen binding domain are each a Fab molecule. In one such aspect, the first antigen binding domain is fused at the C-terminus of the Fab heavy chain to the N-terminus of the Fab heavy chain of the second antigen binding domain. In another such aspect, the second antigen binding domain is fused at the C-terminus of the Fab heavy chain to the N-terminus of the Fab heavy chain of the first antigen binding domain. In aspects wherein either (i) the first antigen binding domain is fused at the C-terminus of the Fab heavy chain to the N-terminus of the Fab heavy chain of the second antigen binding domain or (ii) the second antigen binding domain is fused at the C-terminus of the Fab heavy chain to the N-terminus of the Fab heavy chain of the first antigen binding domain, additionally the Fab light chain of the first antigen binding domain and the Fab light chain of the second antigen binding domain may be fused to each other, optionally via a peptide linker.

A (multispecific) antibody with a single antigen binding domain (such as a Fab molecule) capable of specific binding to a second antigen, e.g. a target cell antigen such as HER2, (for example as shown in FIG. 1A, FIG. 1D, FIG. 1G, FIG. 1H, FIG. 1K, FIG. 1L) is useful, particularly in cases where internalization of the second antigen is to be expected following binding of a high affinity antigen binding domain. In such cases, the presence of more than one antigen binding domain specific for the second antigen may enhance internalization of the second antigen, thereby reducing its availability.

In other cases, however, it will be advantageous to have a (multispecific) antibody comprising two or more antigen binding domains (such as Fab molecules) specific for a second antigen, e.g. a target cell antigen (see examples shown in FIG. 1B, FIG. 1C, FIG. 1E, FIG. 1F, FIG. 1I, FIG. 1J, FIG. 1M or FIG. 1N), for example to optimize targeting to the target site or to allow crosslinking of target cell antigens.

Accordingly, in preferred aspects, the (multispecific) antibody according to the present invention comprises a third antigen binding domain.

In one aspect, the third antigen binding domain binds to the second antigen, e.g. a target cell antigen such as HER2. In one aspect, the third antigen binding domain is a Fab molecule.

In one aspect, the third antigen binding domain is identical to the second antigen binding domain.

In some aspects, the third and the second antigen binding domain are each a Fab molecule and the third antigen binding domain is identical to the second antigen binding domain. Thus, in these aspects, the second and the third antigen binding domain comprise the same heavy and light chain amino acid sequences and have the same arrangement of domains (i.e. conventional or crossover). Furthermore, in these aspects, the third antigen binding domain comprises the same amino acid substitutions, if any, as the second antigen binding domain. For example, the amino acid substitutions described herein as “charge modifications” will be made in the constant domain CL and the constant domain CH1 of each of the second antigen binding domain and the third antigen binding domain. Alternatively, said amino acid substitutions may be made in the constant domain CL and the constant domain CH1 of the first antigen binding domain (which in preferred aspects is also a Fab molecule), but not in the constant domain CL and the constant domain CH1 of the second antigen binding domain and the third antigen binding domain.

Like the second antigen binding domain, the third antigen binding domain preferably is a conventional Fab molecule. Aspects wherein the second and the third antigen binding domains are crossover Fab molecules (and the first antigen binding domain is a conventional Fab molecule) are, however, also contemplated. Thus, in preferred aspects, the second and the third antigen binding domains are each a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In other aspects, the second and the third antigen binding domains are each a crossover Fab molecule and the first antigen binding domain is a conventional Fab molecule.

If a third antigen binding domain is present, in a preferred aspect the first antigen domain binds to CD3, and the second and third antigen binding domain bind to a second antigen, particularly a target cell antigen, such as HER2.

In preferred aspects, the (multispecific) antibody of the invention comprises an Fc domain composed of a first and a second subunit. The first and the second subunit of the Fc domain are capable of stable association.

The (multispecific) antibody according to the invention can have different configurations, i.e. the first, second (and optionally third) antigen binding domain may be fused to each other and to the Fc domain in different ways. The components may be fused to each other directly or, preferably, via one or more suitable peptide linkers. Where fusion of a Fab molecule is to the N-terminus of a subunit of the Fc domain, it is typically via an immunoglobulin hinge region.

In some aspects, the first and the second antigen binding domain are each a Fab molecule and the first antigen binding domain is fused at the C-terminus of the Fab heavy chain to the N-terminus of the first or the second subunit of the Fc domain. In such aspects, the second antigen binding domain may be fused at the C-terminus of the Fab heavy chain to the N-terminus of the Fab heavy chain of the first antigen binding domain or to the N-terminus of the other one of the subunits of the Fc domain. In preferred such aspects, the second antigen binding domain is a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In other such aspects, the second antigen binding domain is a crossover Fab molecule and the first antigen binding domain is a conventional Fab molecule.

In one aspect, the first and the second antigen binding domain are each a Fab molecule, the first antigen binding domain is fused at the C-terminus of the Fab heavy chain to the N-terminus of the first or the second subunit of the Fc domain, and the second antigen binding domain is fused at the C-terminus of the Fab heavy chain to the N-terminus of the Fab heavy chain of the first antigen binding domain. In a specific aspect, the (multispecific) antibody essentially consists of the first and the second Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first or the second subunit of the Fc domain. Such a configuration is schematically depicted in FIG. 1G and FIG. 1K (with the first antigen binding domain in these examples being a VH/VL crossover Fab molecule). Optionally, the Fab light chain of the first Fab molecule and the Fab light chain of the second Fab molecule may additionally be fused to each other.

In another aspect, the first and the second antigen binding domain are each a Fab molecule and the first and the second antigen binding domain are each fused at the C-terminus of the Fab heavy chain to the N-terminus of one of the subunits of the Fc domain. In a specific aspect, the (multispecific) antibody essentially consists of the first and the second Fab molecule, the Fc domain composed of a first and a second subunit, and optionally one or more peptide linkers, wherein the first and the second Fab molecule are each fused at the C-terminus of the Fab heavy chain to the N-terminus of one of the subunits of the Fc domain. Such a configuration is schematically depicted in FIG. 1A and FIG. 1D (in these examples with the first antigen binding domain being a VH/VL crossover Fab molecule and the second antigen binding domain being a conventional Fab molecule). The first and the second Fab molecule may be fused to the Fc domain directly or through a peptide linker. In a preferred aspect the first and the second Fab molecule are each fused to the Fc domain through an immunoglobulin hinge region. In a specific aspect, the immunoglobulin hinge region is a human IgG₁ hinge region, particularly where the Fc domain is an IgG₁ Fc domain.

In some aspects, the first and the second antigen binding domain are each a Fab molecule and the second antigen binding domain is fused at the C-terminus of the Fab heavy chain to the N-terminus of the first or the second subunit of the Fc domain. In such aspects, the first antigen binding domain may be fused at the C-terminus of the Fab heavy chain to the N-terminus of the Fab heavy chain of the first antigen binding domain or (as described above) to the N-terminus of the other one of the subunits of the Fc domain. In preferred such aspects, said second antigen binding domain is a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In other such aspects, said second antigen binding domain is a crossover Fab molecule and the first antigen binding domain is a conventional Fab molecule.

In one aspect, the first and the second antigen binding domain are each a Fab molecule, the second antigen binding domain is fused at the C-terminus of the Fab heavy chain to the N-terminus of the first or the second subunit of the Fc domain, and the first antigen binding domain is fused at the C-terminus of the Fab heavy chain to the N-terminus of the Fab heavy chain of the second antigen binding domain. In a specific aspect, the (multispecific) antibody essentially consists of the first and the second Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first or the second subunit of the Fc domain. Such a configuration is schematically depicted in FIG. 1H and FIG. 1L (in these examples with the first antigen binding domain being a VH/VL crossover Fab molecule and the second antigen binding domain being a conventional Fab molecule). Optionally, the Fab light chain of the first Fab molecule and the Fab light chain of the second Fab molecule may additionally be fused to each other.

In some aspects, a third antigen binding domain, particularly a third Fab molecule, is fused at the C-terminus of the Fab heavy chain to the N-terminus of the first or second subunit of the Fc domain. In preferred such aspects, said second and third antigen binding domains are each a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In other such aspects, said second and third antigen binding domains are each a crossover Fab molecule and the first antigen binding domain is a conventional Fab molecule.

In a preferred such aspect, the first and the third antigen binding domain are each fused at the C-terminus of the Fab heavy chain to the N-terminus of one of the subunits of the Fc domain, and the second antigen binding domain is fused at the C-terminus of the Fab heavy chain to the N-terminus of the Fab heavy chain of the first Fab molecule. In a specific aspect, the (multispecific) antibody essentially consists of the first, the second and the third Fab molecule, the Fc domain composed of a first and a second subunit, and optionally one or more peptide linkers, wherein the second Fab molecule is fused at the C-terminus of the Fab heavy chain to the N-terminus of the Fab heavy chain of the first Fab molecule, and the first Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 1B and FIG. 1E (in these examples with the first antigen binding domain being a VH/VL crossover Fab molecule, and the second and the third antigen binding domain being a conventional Fab molecule), and FIG. 1J and FIG. 1N (in these examples with the first antigen binding domain being a conventional Fab molecule, and the second and the third antigen binding domain being a VH/VL crossover Fab molecule). The first and the third Fab molecule may be fused to the Fc domain directly or through a peptide linker. In a preferred aspect, the first and the third Fab molecule are each fused to the Fc domain through an immunoglobulin hinge region. In a specific aspect, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In another such aspect, the second and the third antigen binding domain are each fused at the C-terminus of the Fab heavy chain to the N-terminus of one of the subunits of the Fc domain, and the first antigen binding domain is fused at the C-terminus of the Fab heavy chain to the N-terminus of the Fab heavy chain of the second antigen binding domain. In a specific aspect, the (multispecific) antibody essentially consists of the first, the second and the third Fab molecule, the Fc domain composed of a first and a second subunit, and optionally one or more peptide linkers, wherein the first Fab molecule is fused at the C-terminus of the Fab heavy chain to the N-terminus of the Fab heavy chain of the second Fab molecule, and the second Fab molecule is fused at the C-terminus of the Fab heavy chain to the N-terminus of the first subunit of the Fc domain, and wherein the third Fab molecule is fused at the C-terminus of the Fab heavy chain to the N-terminus of the second subunit of the Fc domain. Such a configuration is schematically depicted in FIG. 1C and FIG. 1F (in these examples with the first antigen binding domain being a VH/VL crossover Fab molecule, and the second and the third antigen binding domain being a conventional Fab molecule) and in FIG. 1I and FIG. 1M (in these examples with the first antigen binding domain being a conventional Fab molecule, and the second and the third antigen binding domain being a VH/VL crossover Fab molecule). The second and the third Fab molecule may be fused to the Fc domain directly or through a peptide linker. In a preferred aspect the second and the third Fab molecule are each fused to the Fc domain through an immunoglobulin hinge region. In a specific aspect, the immunoglobulin hinge region is a human IgG₁ hinge region, particularly where the Fc domain is an IgG₁ Fc domain. Optionally, the Fab light chain of the first Fab molecule and the Fab light chain of the second Fab molecule may additionally be fused to each other.

In configurations of the (multispecific) antibody wherein a Fab molecule is fused at the C-terminus of the Fab heavy chain to the N-terminus of each of the subunits of the Fc domain through an immunoglobulin hinge region, the two Fab molecules, the hinge regions and the Fc domain essentially form an immunoglobulin molecule. In a preferred aspect the immunoglobulin molecule is an IgG class immunoglobulin. In an even more preferred aspect the immunoglobulin is an IgG₁ subclass immunoglobulin. In another aspect the immunoglobulin is an IgG₄ subclass immunoglobulin. In a further preferred aspect the immunoglobulin is a human immunoglobulin. In other aspects the immunoglobulin is a chimeric immunoglobulin or a humanized immunoglobulin. In one aspect, the immunoglobulin comprises a human constant region, particularly a human Fc region.

In some of the (multispecific) antibodies of the invention, the Fab light chain of the first Fab molecule and the Fab light chain of the second Fab molecule are fused to each other, optionally via a peptide linker. Depending on the configuration of the first and the second Fab molecule, the Fab light chain of the first Fab molecule may be fused at its C-terminus to the N-terminus of the Fab light chain of the second Fab molecule, or the Fab light chain of the second Fab molecule may be fused at its C-terminus to the N-terminus of the Fab light chain of the first Fab molecule. Fusion of the Fab light chains of the first and the second Fab molecule further reduces mispairing of unmatched Fab heavy and light chains, and also reduces the number of plasmids needed for expression of some of the (multispecific) antibody of the invention.

The antigen binding domains may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ, G4 (SG₄)ₙ or (G₄S)ₙG₅ peptide linkers. “n” is generally an integer from 1 to 10, typically from 1 to 4. In one aspect said peptide linker has a length of at least 5 amino acids, in one aspect a length of 5 to 100, in a further aspect of 10 to 50 amino acids. In one aspect said peptide linker is (GxS)ₙ or (GxS)ₙGₘ with G=glycine, S=serine, and (x=3, n=3, 4, 5 or 6, and m=0, 1, 2 or 3) or (x=4, n=1, 2, 3, 4 or 5 and m=0, 1, 2, 3, 4 or 5), in one aspect x=4 and n=2 or 3, in a further aspect x=4 and n=2, in yet a further aspect x=4, n=1 and m=5. In one aspect said peptide linker is (G₄S) 2. In another aspect, said peptide linker is G₄SG₅. In still another aspect, said peptide linker is G₄SG₄. A particularly suitable peptide linker for fusing the Fab light chains of the first and the second Fab molecule to each other is G₄SG₄. An exemplary peptide linker suitable for connecting the Fab heavy chains of the first and the second Fab fragments comprises the sequence (D)-(G₄S)₂ (SEQ ID NOs 42 and 43). Another suitable such linker comprises the sequence (D)-G₄SG₅ (SEQ ID NOs 44 and 45). A particular such linker comprises the sequence G₄SG₄ (SEQ ID NO: 48).

Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In certain aspects the (multispecific) antibody according to the invention comprises a polypeptide wherein the Fab light chain variable region of the first Fab molecule shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region), which in turn shares a carboxy-terminal peptide bond with an Fc domain subunit (VL₍₁₎-CH1₍₁₎-CH2-CH3(-CH4)), and a polypeptide wherein the Fab heavy chain of the second Fab molecule shares a carboxy-terminal peptide bond with an Fc domain subunit (VH₍₂₎-CH1₍₂₎-CH2-CH3(-CH4)). In some aspects the (multispecific) antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎). In certain aspects the polypeptides are covalently linked, e.g., by a disulfide bond.

In certain aspects the (multispecific) antibody according to the invention comprises a polypeptide wherein the Fab heavy chain variable region of the first Fab molecule shares a carboxy-terminal peptide bond with the Fab light chain constant region of the first Fab molecule (i.e. the first Fab molecule comprises a crossover Fab heavy chain, wherein the heavy chain constant region is replaced by a light chain constant region), which in turn shares a carboxy-terminal peptide bond with an Fc domain subunit (VH₍₁₎-CL₍₁₎-CH2-CH3(-CH4)), and a polypeptide wherein the Fab heavy chain of the second Fab molecule shares a carboxy-terminal peptide bond with an Fc domain subunit (VH₍₂₎-CH1₍₂₎-CH2-CH3(-CH4)). In some aspects the (multispecific) antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎). In certain aspects the polypeptides are covalently linked, e.g., by a disulfide bond.

In some aspects, the (multispecific) antibody comprises a polypeptide wherein the Fab light chain variable region of the first Fab molecule shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region), which in turn shares a carboxy-terminal peptide bond with the Fab heavy chain of the second Fab molecule, which in turn shares a carboxy-terminal peptide bond with an Fc domain subunit (VL₍₁₎-CH1₍₁₎-VH₍₂₎-CH1₍₂₎-CH2-CH3(-CH4)). In other aspects, the (multispecific) antibody comprises a polypeptide wherein the Fab heavy chain of the second Fab molecule shares a carboxy-terminal peptide bond with the Fab light chain variable region of the first Fab molecule which in turn shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region), which in turn shares a carboxy-terminal peptide bond with an Fc domain subunit (VH₍₂₎-CH1₍₂₎-VL₍₁₎-CH1₍₁₎-CH2-CH3(-CH4)). In some of these aspects the (multispecific) antibody further comprises a crossover Fab light chain polypeptide of the first Fab molecule, wherein the Fab heavy chain variable region of the first Fab molecule shares a carboxy-terminal peptide bond with the Fab light chain constant region of the first Fab molecule (VH₍₁₎-CL₍₁₎), and the Fab light chain polypeptide of the second Fab molecule (VL₍₂₎-CL₍₂₎). In others of these aspects the (multispecific) antibody further comprises a polypeptide wherein the Fab heavy chain variable region of the first Fab molecule shares a carboxy-terminal peptide bond with the Fab light chain constant region of the first Fab molecule which in turn shares a carboxy-terminal peptide bond with the Fab light chain polypeptide of the second Fab molecule (VH₍₁₎-CL₍₁₎-VL₍₂₎-CL₍₂₎), or a polypeptide wherein the Fab light chain polypeptide of the second Fab molecule shares a carboxy-terminal peptide bond with the Fab heavy chain variable region of the first Fab molecule which in turn shares a carboxy-terminal peptide bond with the Fab light chain constant region of the first Fab molecule (VL₍₂₎-CL₍₂₎-VH₍₁₎-CL₍₁₎), as appropriate. The (multispecific) antibody according to these aspects may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain aspects the polypeptides are covalently linked, e.g., by a disulfide bond.

In some aspects, the (multispecific) antibody comprises a polypeptide wherein the Fab heavy chain variable region of the first Fab molecule shares a carboxy-terminal peptide bond with the Fab light chain constant region of the first Fab molecule (i.e. the first Fab molecule comprises a crossover Fab heavy chain, wherein the heavy chain constant region is replaced by a light chain constant region), which in turn shares a carboxy-terminal peptide bond with the Fab heavy chain of the second Fab molecule, which in turn shares a carboxy-terminal peptide bond with an Fc domain subunit (VH₍₁₎-CL₍₁₎-VH₍₂₎-CH1₍₂₎-CH2-CH3(-CH4)). In other aspects, the (multispecific) antibody comprises a polypeptide wherein the Fab heavy chain of the second Fab molecule shares a carboxy-terminal peptide bond with the Fab heavy chain variable region of the first Fab molecule which in turn shares a carboxy-terminal peptide bond with the Fab light chain constant region of the first Fab molecule (i.e. the first Fab molecule comprises a crossover Fab heavy chain, wherein the heavy chain constant region is replaced by a light chain constant region), which in turn shares a carboxy-terminal peptide bond with an Fc domain subunit (VH₍₂₎-CH1₍₂₎-VH₍₁₎-CL₍₁₎-CH2-CH3(-CH4)). In some of these aspects the (multispecific) antibody further comprises a crossover Fab light chain polypeptide of the first Fab molecule, wherein the Fab light chain variable region of the first Fab molecule shares a carboxy-terminal peptide bond with the Fab heavy chain constant region of the first Fab molecule (VL₍₁₎-CH1₍₁₎), and the Fab light chain polypeptide of the second Fab molecule (VL₍₂₎-CL₍₂₎). In others of these aspects the (multispecific) antibody further comprises a polypeptide wherein the Fab light chain variable region of the first Fab molecule shares a carboxy-terminal peptide bond with the Fab heavy chain constant region of the first Fab molecule which in turn shares a carboxy-terminal peptide bond with the Fab light chain polypeptide of the second Fab molecule (VL₍₁₎-CH1₍₁₎-VL₍₂₎-CL₍₂₎), or a polypeptide wherein the Fab light chain polypeptide of the second Fab molecule shares a carboxy-terminal peptide bond with the Fab heavy chain variable region of the first Fab molecule which in turn shares a carboxy-terminal peptide bond with the Fab light chain constant region of the first Fab molecule (VL₍₂₎-CL₍₂₎-VH₍₁₎-CL₍₁₎), as appropriate. The (multispecific) antibody according to these aspects may further comprise (i) an Fc domain subunit polypeptide (CH2-CH3(-CH4)), or (ii) a polypeptide wherein the Fab heavy chain of a third Fab molecule shares a carboxy-terminal peptide bond with an Fc domain subunit (VH₍₃₎-CH1₍₃₎-CH2-CH3(-CH4)) and the Fab light chain polypeptide of a third Fab molecule (VL₍₃₎-CL₍₃₎). In certain aspects the polypeptides are covalently linked, e.g., by a disulfide bond.

In certain aspects, the (multispecific) antibody does not comprise an Fc domain. In preferred such aspects, said second and, if present, third antigen binding domains are each a conventional Fab molecule, and the first antigen binding domain is a crossover Fab molecule as described herein, i.e. a Fab molecule wherein the variable domains VH and VL or the constant domains CL and CH1 of the Fab heavy and light chains are exchanged/replaced by each other. In other such aspects, said second and, if present, third antigen binding domains are each a crossover Fab molecule and the first antigen binding domain is a conventional Fab molecule.

In one such aspect, the (multispecific) antibody essentially consists of the first and the second antigen binding domain, and optionally one or more peptide linkers, wherein the first and the second antigen binding domain are both Fab molecules and the second antigen binding domain is fused at the C-terminus of the Fab heavy chain to the N-terminus of the Fab heavy chain of the first antigen binding domain. Such a configuration is schematically depicted in FIG. 1O and FIG. 1S (in these examples with the first antigen binding domain being a VH/VL crossover Fab molecule and the second antigen binding domain being a conventional Fab molecule).

In another such aspect, the (multispecific) antibody essentially consists of the first and the second antigen binding domain, and optionally one or more peptide linkers, wherein the first and the second antigen binding domain are both Fab molecules and the first antigen binding domain is fused at the C-terminus of the Fab heavy chain to the N-terminus of the Fab heavy chain of the second antigen binding domain. Such a configuration is schematically depicted in FIG. 1P and FIG. 1T (in these examples with the first antigen binding domain being a VH/VL crossover Fab molecule and the second antigen binding domain being a conventional Fab molecule).

In some aspects, the second Fab molecule is fused at the C-terminus of the Fab heavy chain to the N-terminus of the Fab heavy chain of the first Fab molecule, and the (multispecific) antibody further comprises a third antigen binding domain, particularly a third Fab molecule, wherein said third Fab molecule is fused at the C-terminus of the Fab heavy chain to the N-terminus of the Fab heavy chain of the second Fab molecule. In certain such aspects, the (multispecific) antibody essentially consists of the first, the second and the third Fab molecule, and optionally one or more peptide linkers, wherein the second Fab molecule is fused at the C-terminus of the Fab heavy chain to the N-terminus of the Fab heavy chain of the first Fab molecule, and the third Fab molecule is fused at the C-terminus of the Fab heavy chain to the N-terminus of the Fab heavy chain of the second Fab molecule. Such a configuration is schematically depicted in FIG. 1Q and FIG. 1U (in these examples with the first antigen binding domain being a VH/VL crossover Fab molecule and the second and the third antigen binding domain each being a conventional Fab molecule), or FIG. 1X and FIG. 1Z (in these examples with the first antigen binding domain being a conventional Fab molecule and the second and the third antigen binding domain each being a VH/VL crossover Fab molecule).

In some aspects, the first Fab molecule is fused at the C-terminus of the Fab heavy chain to the N-terminus of the Fab heavy chain of the second Fab molecule, and the (multispecific) antibody further comprises a third antigen binding domain, particularly a third Fab molecule, wherein said third Fab molecule is fused at the N-terminus of the Fab heavy chain to the C-terminus of the Fab heavy chain of the second Fab molecule. In certain such aspects, the (multispecific) antibody essentially consists of the first, the second and the third Fab molecule, and optionally one or more peptide linkers, wherein the first Fab molecule is fused at the C-terminus of the Fab heavy chain to the N-terminus of the Fab heavy chain of the second Fab molecule, and the third Fab molecule is fused at the N-terminus of the Fab heavy chain to the C-terminus of the Fab heavy chain of the second Fab molecule. Such a configuration is schematically depicted in FIG. 1R and FIG. 1V (in these examples with the first antigen binding domain being a VH/VL crossover Fab molecule and the second and the third antigen binding domain each being a conventional Fab molecule), or FIG. 1W and FIG. 1Y (in these examples with the first antigen binding domain being a conventional Fab molecule and the second and the third antigen binding domain each being a VH/VL crossover Fab molecule).

In certain aspects the (multispecific) antibody according to the invention comprises a polypeptide wherein the Fab heavy chain of the second Fab molecule shares a carboxy-terminal peptide bond with the Fab light chain variable region of the first Fab molecule, which in turn shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region) (VH₍₂₎-CH1₍₂₎-VL₍₁₎-CH1₍₁₎). In some aspects the (multispecific) antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎).

In certain aspects the (multispecific) antibody according to the invention comprises a polypeptide wherein the Fab light chain variable region of the first Fab molecule shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region), which in turn shares a carboxy-terminal peptide bond with the Fab heavy chain of the second Fab molecule (VL₍₁₎-CH1₍₁₎-VH₍₂₎-CH1₍₂₎). In some aspects the (multispecific) antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎).

In certain aspects the (multispecific) antibody according to the invention comprises a polypeptide wherein the Fab heavy chain of the second Fab molecule shares a carboxy-terminal peptide bond with the Fab heavy chain variable region of the first Fab molecule, which in turn shares a carboxy-terminal peptide bond with the Fab light chain constant region of the first Fab molecule (i.e. the first Fab molecule comprises a crossover Fab heavy chain, wherein the heavy chain constant region is replaced by a light chain constant region) (VH₍₂₎-CH1₍₂₎-VH₍₁₎-CL₍₁₎). In some aspects the (multispecific) antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎).

In certain aspects the (multispecific) antibody according to the invention comprises a polypeptide wherein the Fab heavy chain variable region of the first Fab molecule shares a carboxy-terminal peptide bond with the Fab light chain constant region of the first Fab molecule (i.e. the first Fab molecule comprises a crossover Fab heavy chain, wherein the heavy chain constant region is replaced by a light chain constant region), which in turn shares a carboxy-terminal peptide bond with the Fab heavy chain of the second Fab molecule (VH₍₁₎-CL₍₁₎-VH₍₂₎-CH1₍₂₎). In some aspects the (multispecific) antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎).

In certain aspects the (multispecific) antibody according to the invention comprises a polypeptide wherein the Fab heavy chain of a third Fab molecule shares a carboxy-terminal peptide bond with the Fab heavy chain of the second Fab molecule, which in turn shares a carboxy-terminal peptide bond with the Fab light chain variable region of the first Fab molecule, which in turn shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region) (VH₍₃₎-CH1₍₃₎-VH₍₂₎-CH1₍₂₎-VL₍₁₎-CH1₍₁₎). In some aspects the (multispecific) antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎). In some aspects the (multispecific) antibody further comprises the Fab light chain polypeptide of a third Fab molecule (VL₍₃₎-CL₍₃₎).

In certain aspects the (multispecific) antibody according to the invention comprises a polypeptide wherein the Fab heavy chain of a third Fab molecule shares a carboxy-terminal peptide bond with the Fab heavy chain of the second Fab molecule, which in turn shares a carboxy-terminal peptide bond with the Fab heavy chain variable region of the first Fab molecule, which in turn shares a carboxy-terminal peptide bond with the Fab light chain constant region of the first Fab molecule (i.e. the first Fab molecule comprises a crossover Fab heavy chain, wherein the heavy chain constant region is replaced by a light chain constant region) (VH₍₃₎-CH1₍₃₎-VH₍₂₎-CH1₍₂₎-VH₍₁₎-CL₍₁₎). In some aspects the (multispecific) antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎). In some aspects the (multispecific) antibody further comprises the Fab light chain polypeptide of a third Fab molecule (VL₍₃₎-CL₍₃₎).

In certain aspects the (multispecific) antibody according to the invention comprises a polypeptide wherein the Fab light chain variable region of the first Fab molecule shares a carboxy-terminal peptide bond with the Fab heavy chain constant region of the first Fab molecule (i.e. the first Fab molecule comprises a crossover Fab heavy chain, wherein the heavy chain variable region is replaced by a light chain variable region), which in turn shares a carboxy-terminal peptide bond with the Fab heavy chain of the second Fab molecule, which in turn shares a carboxy-terminal peptide bond with the Fab heavy chain of a third Fab molecule (VL₍₁₎-CH1₍₁₎-VH₍₂₎-CH1₍₂₎-VH₍₃₎-CH1₍₃₎). In some aspects the (multispecific) antibody further comprises a polypeptide wherein the Fab heavy chain variable region of the first Fab molecule shares a carboxy-terminal peptide bond with the Fab light chain constant region of the first Fab molecule (VH₍₁₎-CL₍₁₎) and the Fab light chain polypeptide of the second Fab molecule (VL₍₂₎-CL₍₂₎). In some aspects the (multispecific) antibody further comprises the Fab light chain polypeptide of a third Fab molecule (VL₍₃₎-CL₍₃₎).

In certain aspects the (multispecific) antibody according to the invention comprises a polypeptide wherein the Fab heavy chain variable region of the first Fab molecule shares a carboxy-terminal peptide bond with the Fab light chain constant region of the first Fab molecule (i.e. the first Fab molecule comprises a crossover Fab heavy chain, wherein the heavy chain constant region is replaced by a light chain constant region), which in turn shares a carboxy-terminal peptide bond with the Fab heavy chain of the second Fab molecule, which in turn shares a carboxy-terminal peptide bond with the Fab heavy chain of a third Fab molecule (VH₍₁₎-CL₍₁₎-VH₍₂₎-CH1₍₂₎-VH₍₃₎-CH1₍₃₎). In some aspects the (multispecific) antibody further comprises a polypeptide wherein the Fab light chain variable region of the first Fab molecule shares a carboxy-terminal peptide bond with the Fab heavy chain constant region of the first Fab molecule (VL₍₁₎-CH1₍₁₎) and the Fab light chain polypeptide of the second Fab molecule (VL₍₂₎-CL₍₂₎). In some aspects the (multispecific) antibody further comprises the Fab light chain polypeptide of a third Fab molecule (VL₍₃₎-CL₍₃₎).

In certain aspects the (multispecific) antibody according to the invention comprises a polypeptide wherein the Fab heavy chain of the first Fab molecule shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab light chain variable region of a third Fab molecule, which in turn shares a carboxy-terminal peptide bond with the Fab heavy chain constant region of a third Fab molecule (i.e. the third Fab molecule comprises a crossover Fab heavy chain, wherein the heavy chain variable region is replaced by a light chain variable region) (VH₍₁₎-CH1₍₁₎-VL₍₂₎-CH1₍₂₎-VL₍₃₎-CH1₍₃₎). In some aspects the (multispecific) antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some aspects the (multispecific) antibody further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain aspects the (multispecific) antibody according to the invention comprises a polypeptide wherein the Fab heavy chain of the first Fab molecule shares a carboxy-terminal peptide bond with the Fab heavy chain variable region of the second Fab molecule, which in turn shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain variable region of a third Fab molecule, which in turn shares a carboxy-terminal peptide bond with the Fab light chain constant region of a third Fab molecule (i.e. the third Fab molecule comprises a crossover Fab heavy chain, wherein the heavy chain constant region is replaced by a light chain constant region) (VH₍₁₎-CH1₍₁₎-VH₍₂₎-CL₍₂₎-VH₍₃₎-CL₍₃₎). In some aspects the (multispecific) antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some aspects the (multispecific) antibody further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

In certain aspects the (multispecific) antibody according to the invention comprises a polypeptide wherein the Fab light chain variable region of a third Fab molecule shares a carboxy-terminal peptide bond with the Fab heavy chain constant region of a third Fab molecule (i.e. the third Fab molecule comprises a crossover Fab heavy chain, wherein the heavy chain variable region is replaced by a light chain variable region), which in turn shares a carboxy-terminal peptide bond with the Fab light chain variable region of the second Fab molecule, which in turn shares a carboxy-terminal peptide bond with the Fab heavy chain constant region of the second Fab molecule (i.e. the second Fab molecule comprises a crossover Fab heavy chain, wherein the heavy chain variable region is replaced by a light chain variable region), which in turn shares a carboxy-terminal peptide bond with the Fab heavy chain of the first Fab molecule (VL₍₃₎-CH1₍₃₎-VL₍₂₎-CH1₍₂₎-VH₍₁₎-CH1₍₁₎). In some aspects the (multispecific) antibody further comprises a polypeptide wherein the Fab heavy chain variable region of the second Fab molecule shares a carboxy-terminal peptide bond with the Fab light chain constant region of the second Fab molecule (VH₍₂₎-CL₍₂₎) and the Fab light chain polypeptide of the first Fab molecule (VL₍₁₎-CL₍₁₎). In some aspects the (multispecific) antibody further comprises a polypeptide wherein the Fab heavy chain variable region of a third Fab molecule shares a carboxy-terminal peptide bond with the Fab light chain constant region of a third Fab molecule (VH₍₃₎-CL₍₃₎).

In certain aspects the (multispecific) antibody according to the invention comprises a polypeptide wherein the Fab heavy chain variable region of a third Fab molecule shares a carboxy-terminal peptide bond with the Fab light chain constant region of a third Fab molecule (i.e. the third Fab molecule comprises a crossover Fab heavy chain, wherein the heavy chain constant region is replaced by a light chain constant region), which in turn shares a carboxy-terminal peptide bond with the Fab heavy chain variable region of the second Fab molecule, which in turn shares a carboxy-terminal peptide bond with the Fab light chain constant region of the second Fab molecule (i.e. the second Fab molecule comprises a crossover Fab heavy chain, wherein the heavy chain constant region is replaced by a light chain constant region), which in turn shares a carboxy-terminal peptide bond with the Fab heavy chain of the first Fab molecule (VH₍₃₎-CL₍₃₎-VH₍₂₎-CL₍₂₎-VH₍₁₎-CH1₍₁₎). In some aspects the (multispecific) antibody further comprises a polypeptide wherein the Fab light chain variable region of the second Fab molecule shares a carboxy-terminal peptide bond with the Fab heavy chain constant region of the second Fab molecule (VL₍₂₎-CH1₍₂₎) and the Fab light chain polypeptide of the first Fab molecule (VL₍₁₎-CL₍₁₎). In some aspects the (multispecific) antibody further comprises a polypeptide wherein the Fab light chain variable region of a third Fab molecule shares a carboxy-terminal peptide bond with the Fab heavy chain constant region of a third Fab molecule (VL₍₃₎-CH1₍₃₎).

In one aspect, the invention provides a (multispecific) antibody comprising
(A) a first antigen binding domain that binds to CD3, wherein the first antigen binding domain is a Fab molecule wherein the variable domains VL and VH or the constant domains CL and CH1 of the Fab light chain and the Fab heavy chain are replaced by each other, and comprises (i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7; and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12;
(B) a second antigen binding domain that binds to a second antigen, particularly a target cell antigen, more particularly HER2, wherein the second antigen binding domain is a (conventional) Fab molecule;
(C) an Fc domain composed of a first and a second subunit;
wherein
(i) the first antigen binding domain under (A) is fused at the C-terminus of the Fab heavy chain to the N-terminus of the Fab heavy chain of the second antigen binding domain under (B), and the second antigen binding domain under (B) is fused at the C-terminus of the Fab heavy chain to the N-terminus of one of the subunits of the Fc domain under (C), or
(ii) the second antigen binding domain under (B) is fused at the C-terminus of the Fab heavy chain to the N-terminus of the Fab heavy chain of the first antigen binding domain under (A), and the first antigen binding domain under (A) is fused at the C-terminus of the Fab heavy chain to the N-terminus of one of the subunits of the Fc domain under (C).

In a preferred aspect, the invention provides a (multispecific) antibody comprising
(A) a first antigen binding domain that binds to CD3, wherein the first antigen binding domain is a Fab molecule wherein the variable domains VL and VH or the constant domains CL and CH1 of the Fab light chain and the Fab heavy chain are replaced by each other, and comprises (i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7; and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12;
(B) a second and a third antigen binding domain that bind to a second antigen, particularly a target cell antigen, more particularly HER2, wherein the second and the third antigen binding domain are each a (conventional) Fab molecule; and
(C) an Fc domain composed of a first and a second subunit;
wherein
(i) the first antigen binding domain under (A) is fused at the C-terminus of the Fab heavy chain to the N-terminus of the Fab heavy chain of the second antigen binding domain under (B), and the second antigen binding domain under (B) and the third antigen binding domain under (B) are each fused at the C-terminus of the Fab heavy chain to the N-terminus of one of the subunits of the Fc domain under (C), or
(ii) the second antigen binding domain under (B) is fused at the C-terminus of the Fab heavy chain to the N-terminus of the Fab heavy chain of the first antigen binding domain under (A), and the first antigen binding domain under (A) and the third antigen binding domain under (B) are each fused at the C-terminus of the Fab heavy chain to the N-terminus of one of the subunits of the Fc domain under (C).

In another aspect, the invention provides a (multispecific) antibody comprising
(A) a first antigen binding domain that binds to CD3, wherein the first antigen binding domain is a Fab molecule wherein the variable domains VL and VH or the constant domains CL and CH1 of the Fab light chain and the Fab heavy chain are replaced by each other, and comprises (i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7; and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12;
(B) a second antigen binding domain that binds to a second antigen, particularly a target cell antigen, more particularly HER2, wherein the second antigen binding domain is a (conventional) Fab molecule;
(C) an Fc domain composed of a first and a second subunit;
wherein
(i) the first antigen binding domain under (A) and the second antigen binding domain under (B) are each fused at the C-terminus of the Fab heavy chain to the N-terminus of one of the subunits of the Fc domain under (C).

In all of the different configurations of the (multispecific) antibody according to the invention, the amino acid substitutions (“charge modifications”) described herein, if present, may either be in the CH1 and CL domains of the second and (if present) the third antigen binding domain/Fab molecule, or in the CH1 and CL domains of the first antigen binding domain/Fab molecule. Preferably, they are in the CH1 and CL domains of the second and (if present) the third antigen binding domain/Fab molecule. In accordance with the concept of the invention, if amino acid substitutions as described herein are made in the second (and, if present, the third) antigen binding domain/Fab molecule, no such amino acid substitutions are made in the first antigen binding domain/Fab molecule. Conversely, if amino acid substitutions as described herein are made in the first antigen binding domain/Fab molecule, no such amino acid substitutions are made in the second (and, if present, the third) antigen binding domain/Fab molecule. Amino acid substitutions are preferably made in (multispecific) antibodies comprising a Fab molecule wherein the variable domains VL and VH1 of the Fab light chain and the Fab heavy chain are replaced by each other.

In preferred aspects of the (multispecific) antibody according to the invention, particularly wherein amino acid substitutions as described herein are made in the second (and, if present, the third) antigen binding domain/Fab molecule, the constant domain CL of the second (and, if present, the third) Fab molecule is of kappa isotype. In other aspects of the (multispecific) antibody according to the invention, particularly wherein amino acid substitutions as described herein are made in the first antigen binding domain/Fab molecule, the constant domain CL of the first antigen binding domain/Fab molecule is of kappa isotype. In some aspects, the constant domain CL of the second (and, if present, the third) antigen binding domain/Fab molecule and the constant domain CL of the first antigen binding domain/Fab molecule are of kappa isotype.

In one aspect, the invention provides a (multispecific) antibody comprising
(A) a first antigen binding domain that binds to CD3, wherein the first antigen binding domain is a Fab molecule wherein the variable domains VL and VH of the Fab light chain and the Fab heavy chain are replaced by each other, and comprises (i) a heavy chain variable region 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7; and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12;
(B) a second antigen binding domain that binds to a second antigen, particularly a target cell antigen, more particularly HER2, wherein the second antigen binding domain is a (conventional) Fab molecule;
(C) an Fc domain composed of a first and a second subunit;
wherein in the constant domain CL of the second antigen binding domain under (B) the amino acid at position 124 is substituted by lysine (K) (numbering according to Kabat) and the amino acid at position 123 is substituted by lysine (K) or arginine (R) (numbering according to Kabat) (most preferably by arginine (R)), and wherein in the constant domain CH1 of the second antigen binding domain under (B) the amino acid at position 147 is substituted by glutamic acid (E) (numbering according to Kabat EU index) and the amino acid at position 213 is substituted by glutamic acid (E) (numbering according to Kabat EU index); and
wherein
(i) the first antigen binding domain under (A) is fused at the C-terminus of the Fab heavy chain to the N-terminus of the Fab heavy chain of the second antigen binding domain under (B), and the second antigen binding domain under (B) is fused at the C-terminus of the Fab heavy chain to the N-terminus of one of the subunits of the Fc domain under (C), or
(ii) the second antigen binding domain under (B) is fused at the C-terminus of the Fab heavy chain to the N-terminus of the Fab heavy chain of the first antigen binding domain under (A), and the first antigen binding domain under (A) is fused at the C-terminus of the Fab heavy chain to the N-terminus of one of the subunits of the Fc domain under (C).

In a preferred aspect, the invention provides a (multispecific) antibody comprising
(A) a first antigen binding domain that binds to CD3, wherein the first antigen binding domain is a Fab molecule wherein the variable domains VL and VH of the Fab light chain and the Fab heavy chain are replaced by each other, and comprises (i) a heavy chain variable region 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7; and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12;
(B) a second and a third antigen binding domain that bind to a second antigen, particularly a target cell antigen, more particularly HER2, wherein the second and third antigen binding domain are each a (conventional) Fab molecule; and
(C) an Fc domain composed of a first and a second subunit;
wherein in the constant domain CL of the second antigen binding domain under (B) and the third antigen binding domain under (B) the amino acid at position 124 is substituted by lysine (K) (numbering according to Kabat) and the amino acid at position 123 is substituted by lysine (K) or arginine (R) (numbering according to Kabat) (most preferably by arginine (R)), and wherein in the constant domain CH1 of the second antigen binding domain under (B) and the third antigen binding domain under (B) the amino acid at position 147 is substituted by glutamic acid (E) (numbering according to Kabat EU index) and the amino acid at position 213 is substituted by glutamic acid (E) (numbering according to Kabat EU index); and wherein
(i) the first antigen binding domain under (A) is fused at the C-terminus of the Fab heavy chain to the N-terminus of the Fab heavy chain of the second antigen binding domain under (B), and the second antigen binding domain under (B) and the third antigen binding domain under (B) are each fused at the C-terminus of the Fab heavy chain to the N-terminus of one of the subunits of the Fc domain under (C), or
(ii) the second antigen binding domain under (B) is fused at the C-terminus of the Fab heavy chain to the N-terminus of the Fab heavy chain of the first antigen binding domain under (A), and the first antigen binding domain under (A) and the third antigen binding domain under (B) are each fused at the C-terminus of the Fab heavy chain to the N-terminus of one of the subunits of the Fc domain under (C).

In another aspect, the invention provides a (multispecific) antibody comprising
(A) a first antigen binding domain that binds to CD3, wherein the first antigen binding domain is a Fab molecule wherein the variable domains VL and VH of the Fab light chain and the Fab heavy chain are replaced by each other, and comprises (i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7; and/or (ii) a VH comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a VL comprising the amino acid sequence of SEQ ID NO: 12;
(B) a second antigen binding domain that binds to a second antigen, particularly a target cell antigen, more particularly HER2, wherein the second antigen binding domain is a (conventional) Fab molecule;
(C) an Fc domain composed of a first and a second subunit;
wherein in the constant domain CL of the second antigen binding domain under (B) the amino acid at position 124 is substituted by lysine (K) (numbering according to Kabat) and the amino acid at position 123 is substituted by lysine (K) or arginine (R) (numbering according to Kabat) (most preferably by arginine (R)), and wherein in the constant domain CH1 of the second antigen binding domain under (B) the amino acid at position 147 is substituted by glutamic acid (E) (numbering according to Kabat EU index) and the amino acid at position 213 is substituted by glutamic acid (E) (numbering according to Kabat EU index); and
wherein the first antigen binding domain under (A) and the second antigen binding domain under (B) are each fused at the C-terminus of the Fab heavy chain to the N-terminus of one of the subunits of the Fc domain under (C).

According to any of the above aspects, components of the (multispecific) antibody (e.g. Fab molecules, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ, G₄(SG₄)ₙ or (G₄S)ₙG₅ peptide linkers, wherein n is generally an integer from 1 to 10, typically from 1 to 4.

In one aspect, the invention provides a (multispecific) antibody comprising
(A) a first antigen binding domain that binds CD3, wherein the first antigen binding domain is a Fab molecule wherein the variable domains VL and VH of the Fab light chain and the Fab heavy chain are replaced by each other, and comprises a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3, and a light chain variable region (VL) comprising a light chain complementarity determining region (LCDR) 1 of SEQ ID NO: 5, a LCDR 2 of SEQ ID NO: 11 and a LCDR 3 of SEQ ID NO: 7;
(B) a second and a third antigen binding domain that bind to HER2, wherein the second and the third antigen binding domain are each a (conventional) Fab molecule, and comprise a heavy chain variable region (VH) comprising a heavy chain complementarity determining region (HCDR) 1 of SEQ ID NO: 20, a HCDR 2 of SEQ ID NO: 21, and a HCDR 3 of SEQ ID NO: 22, and a light chain variable region (VL) comprising a light chain complementarity determining region (LCDR) 1 of SEQ ID NO: 24, a LCDR 2 of SEQ ID NO: 25 and a LCDR 3 of SEQ ID NO: 26;
(C) an Fc domain composed of a first and a second subunit;
wherein
in the constant domain CL of the second and the third antigen binding domain under (B) the amino acid at position 124 is substituted by lysine (K) (numbering according to Kabat) and the amino acid at position 123 is substituted by lysine (K) or arginine (R) (numbering according to Kabat) (most preferably by arginine (R)), and wherein in the constant domain CH1 of the second and the third antigen binding domain under (B) the amino acid at position 147 is substituted by glutamic acid (E) (numbering according to Kabat EU index) and the amino acid at position 213 is substituted by glutamic acid (E) (numbering according to Kabat EU index);
and wherein further
the second antigen binding domain under (B) is fused at the C-terminus of the Fab heavy chain to the N-terminus of the Fab heavy chain of the first antigen binding domain under (A), and the first antigen binding domain under (A) and the third antigen binding domain under (B) are each fused at the C-terminus of the Fab heavy chain to the N-terminus of one of the subunits of the Fc domain under (C).

In a further aspect, the invention provides a (multispecific) antibody comprising
(A) a first antigen binding domain that binds to CD3, wherein the first antigen binding domain is a Fab molecule wherein the variable domains VL and VH of the Fab light chain and the Fab heavy chain are replaced by each other, and comprises a heavy chain variable region comprising an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and a light chain variable region comprising the amino acid sequence of SEQ ID NO: 12;
(B) a second and a third antigen binding domain that bind to HER2, wherein the second and the third antigen binding domain are each a (conventional) Fab molecule, and comprise a heavy chain variable region comprising the amino acid sequence of SEQ ID NO: 23 and a light chain variable region comprising the amino acid sequence of SEQ ID NO: 27;
(C) an Fc domain composed of a first and a second subunit;
wherein
in the constant domain CL of the second and the third antigen binding domain under (B) the amino acid at position 124 is substituted by lysine (K) (numbering according to Kabat) and the amino acid at position 123 is substituted by lysine (K) or arginine (R) (numbering according to Kabat) (most preferably by arginine (R)), and wherein in the constant domain CH1 of the second and the third antigen binding domain under (B) the amino acid at position 147 is substituted by glutamic acid (E) (numbering according to Kabat EU index) and the amino acid at position 213 is substituted by glutamic acid (E) (numbering according to Kabat EU index);
and wherein further
the second antigen binding domain under (B) is fused at the C-terminus of the Fab heavy chain to the N-terminus of the Fab heavy chain of the first antigen binding domain under (A), and the first antigen binding domain under (A) and the third antigen binding domain under (B) are each fused at the C-terminus of the Fab heavy chain to the N-terminus of one of the subunits of the Fc domain under (C).

In one aspect according to these aspects of the invention, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index).

In a further aspect according to these aspects of the invention, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index).

In still a further aspect according to these aspects of the invention, in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In still a further aspect according to these aspects of the invention, the Fc domain is a human IgG₁ Fc domain.

In a specific aspect, the (multispecific) antibody comprises a polypeptide comprising an amino acid sequence that is at least 95%, 96%, 97%, 98%, or 99% identical to a sequence selected from the group consisting of SEQ ID NO: 32, SEQ ID NO: 33, and SEQ ID NO: 34, a polypeptide (particularly two polypeptides) comprising an amino acid sequence that is at least 95%, 96%, 97%, 98%, or 99% identical to the sequence of SEQ ID NO: 31, a polypeptide comprising an amino acid sequence that is at least 95%, 96%, 97%, 98%, or 99% identical to the sequence of SEQ ID NO: 46, and a polypeptide comprising an amino acid sequence that is at least 95%, 96%, 97%, 98%, or 99% identical to the sequence of SEQ ID NO: 29. In a further specific aspect, the (multispecific) antibody comprises a polypeptide comprising an amino acid sequence selected from the group consisting of SEQ ID NO: 32, SEQ ID NO: 33, and SEQ ID NO: 34, a polypeptide (particularly two polypeptides) comprising the amino acid sequence of SEQ ID NO: 31, a polypeptide comprising the amino acid sequence of SEQ ID NO: 46, and a polypeptide comprising the amino acid sequence of SEQ ID NO: 29.

In one aspect the invention provides a (multispecific) antibody that binds to CD3 and HER2, comprising a polypeptide comprising an amino acid sequence that is at least 95%, 96%, 97%, 98%, or 99% identical to a sequence selected from the group consisting of SEQ ID NO: 32, SEQ ID NO: 33, and SEQ ID NO: 34, a polypeptide (particularly two polypeptides) comprising an amino acid sequence that is at least 95%, 96%, 97%, 98%, or 99% identical to the sequence of SEQ ID NO: 31, a polypeptide comprising an amino acid sequence that is at least 95%, 96%, 97%, 98%, or 99% identical to the sequence of SEQ ID NO: 46, and a polypeptide comprising an amino acid sequence that is at least 95%, 96%, 97%, 98%, or 99% identical to the sequence of SEQ ID NO: 29. In one aspect the invention provides a (multispecific) antibody that binds to CD3 and HER2, comprising a polypeptide comprising an amino acid sequence selected from the group consisting of SEQ ID NO: 32, SEQ ID NO: 33, and SEQ ID NO: 34, a polypeptide (particularly two polypeptides) comprising the amino acid sequence of SEQ ID NO: 31, a polypeptide comprising the amino acid sequence of SEQ ID NO: 46, and a polypeptide comprising the amino acid sequence of SEQ ID NO: 29.

In one aspect according to these aspects of the invention, the VH of the first antigen binding domain (comprised in SEQ ID NOs 32, 33 and 34) comprises amino acid substitutions L108S and S113G (Kabat numbering). Accordingly, the (multispecific) antibody according to these aspects of the invention may comprise a polypeptide comprising an amino acid sequence of SEQ ID NO: 32, SEQ ID NO: 33 or SEQ ID NO: 34, wherein said polypeptide comprises the above amino acid substitutions. Furthermore, in one aspect according to these aspects of the invention, the amino acid sequence immediately adjacent to the C-terminus of the VL of the first antigen binding domain (comprised in SEQ ID NO: 46) is modified from SSAST to SEAEK (positions 342-346 of SEQ ID NO: 46). Without wishing to be bound by theory, these mutations are predicted to reduce immunogenicity of the binder if placed in a VH/VL crossover Fab molecule. Accordingly, the multispecific according to these aspects of the invention may comprise a polypeptide comprising an amino acid sequence of SEQ ID NO: 46, wherein said polypeptide comprises the above modification. An exemplary such (multispecific) antibody that binds to CD3 and HER2 comprises a polypeptide comprising an amino acid sequence that is at least 95%, 96%, 97%, 98%, 99% or 100% identical to the sequence of SEQ ID NO: 35, a polypeptide (particularly two polypeptides) comprising an amino acid sequence that is at least 95%, 96%, 97%, 98%, 99% or 100% identical to the sequence of SEQ ID NO: 31, a polypeptide comprising an amino acid sequence that is at least 95%, 96%, 97%, 98%, 99% or 100% identical to the sequence of SEQ ID NO: 47, and a polypeptide comprising an amino acid sequence that is at least 95%, 96%, 97%, 98%, 99% or 100% identical to the sequence of SEQ ID NO: 29.

### 8. Fc Domain Variants

In preferred aspects, the (multispecific) antibody of the invention comprises an Fc domain composed of a first and a second subunit.

The Fc domain of the (multispecific) antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one aspect, the (multispecific) antibody of the invention comprises not more than one Fc domain.

In one aspect, the Fc domain of the (multispecific) antibody is an IgG Fc domain. In a preferred aspect, the Fc domain is an IgG₁ Fc domain. In another aspect the Fc domain is an IgG₄ Fc domain. In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further preferred aspect, the Fc domain is a human Fc domain. In an even more preferred aspect, the Fc domain is a human IgG₁ Fc domain. An exemplary sequence of a human IgG₁ Fc region is given in SEQ ID NO: 38.

### a) Fc Domain Modifications Promoting Heterodimerization

(Multispecific) antibodies according to the invention comprise different antigen binding domains, which may be fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of (multispecific) antibodies in recombinant production, it will thus be advantageous to introduce in the Fc domain of the (multispecific) antibody a modification promoting the association of the desired polypeptides.

Accordingly, in preferred aspects, the Fc domain of the (multispecific) antibody according to the invention comprises a modification promoting the association of the first and the second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homdimers between the two first or the two second CH3 domains are formed). These different approaches for improved heavy chain heterodimerization are contemplated as different alternatives in combination with the heavy-light chain modifications (e.g. VH and VL exchange/replacement in one binding arm and the introduction of substitutions of charged amino acids with opposite charges in the CH1/CL interface) in the (multispecific) antibody which reduce heavy/light chain mispairing and Bence Jones-type side products.

In a specific aspect said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a preferred aspect, in the CH3 domain of the first subunit of the Fc domain of the (multispecific) antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine(S), threonine (T), and valine (V).

The protuberance and cavity can be made by altering the nucleic acid encoding the polypeptides, e.g. by site-specific mutagenesis, or by peptide synthesis.

In a specific aspect, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In a preferred aspect the antigen binding domain that binds to CD3 is fused (optionally via the second antigen binding domain, that binds to a second antigen, and/or a peptide linker) to the first subunit of the Fc domain (comprising the “knob” modification). Without wishing to be bound by theory, fusion of the antigen binding domain that binds CD3 to the knob-containing subunit of the Fc domain will (further) minimize the generation of antibodies comprising two antigen binding domains that bind to CD3 (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one aspect, the heterodimerization approach described in EP 1870459, is used alternatively. This approach is based on the introduction of charged amino acids with opposite charges at specific amino acid positions in the CH3/CH3 domain interface between the two subunits of the Fc domain. A particular aspect for the (multispecific) antibody of the invention are amino acid mutations R409D; K370E in one of the two CH3 domains (of the Fc domain) and amino acid mutations D399K; E357K in the other one of the CH3 domains of the Fc domain (numbering according to Kabat EU index).

In another aspect, the (multispecific) antibody of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another aspect, the (multispecific) antibody of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said (multispecific) antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one aspect, the heterodimerization approach described in WO 2013/157953 is used alternatively. In one aspect, a first CH3 domain comprises amino acid mutation T366K and a second CH3 domain comprises amino acid mutation L351D (numberings according to Kabat EU index). In a further aspect, the first CH3 domain comprises further amino acid mutation L351K. In a further aspect, the second CH3 domain comprises further an amino acid mutation selected from Y349E, Y349D and L368E (particularly L368E) (numberings according to Kabat EU index).

In one aspect, the heterodimerization approach described in WO 2012/058768 is used alternatively. In one aspect a first CH3 domain comprises amino acid mutations L351Y, Y407A and a second CH3 domain comprises amino acid mutations T366A, K409F. In a further aspect the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F4051, F405M, F405T, F405S, F405V or F405W, c) N390R, N390K or N390D, f) K392V, K392M, K392R, K392L, K392F or K392E (numberings according to Kabat EU index). In a further aspect a first CH3 domain comprises amino acid mutations L351Y, Y407A and a second CH3 domain comprises amino acid mutations T366V, K409F. In a further aspect, a first CH3 domain comprises amino acid mutation Y407A and a second CH3 domain comprises amino acid mutations T366A, K409F. In a further aspect, the second CH3 domain further comprises amino acid mutations K392E, T411E, D399R and S400R (numberings according to Kabat EU index).

In one aspect, the heterodimerization approach described in WO 2011/143545 is used alternatively, e.g. with the amino acid modification at a position selected from the group consisting of 368 and 409 (numbering according to Kabat EU index).

In one aspect, the heterodimerization approach described in WO 2011/090762, which also uses the knobs-into-holes technology described above, is used alternatively. In one aspect a first CH3 domain comprises amino acid mutation T366W and a second CH3 domain comprises amino acid mutation Y407A. In one aspect, a first CH3 domain comprises amino acid mutation T366Y and a second CH3 domain comprises amino acid mutation Y407T (numberings according to Kabat EU index).

In one aspect, the (multispecific) antibody or its Fc domain is of IgG₂ subclass and the heterodimerization approach described in WO 2010/129304 is used alternatively.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such aspect, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In a further aspect, the first CH3 domain further comprises amino acid substitution of K409 or R409 with a negatively charged amino acid (e.g. glutamic acid (E), or aspartic acid (D), particularly K409D or R409D). In a further aspect the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further aspect, the heterodimerization approach described in WO 2007/147901 is used alternatively. In one aspect, a first CH3 domain comprises amino acid mutations K253E, D282K, and K322D and a second CH3 domain comprises amino acid mutations D239K, E240K, and K292D (numberings according to Kabat EU index).

In still another aspect, the heterodimerization approach described in WO 2007/110205 can be used alternatively.

In one aspect, the first subunit of the Fc domain comprises amino acid substitutions K392D and K409D, and the second subunit of the Fc domain comprises amino acid substitutions D356K and D399K (numbering according to Kabat EU index).

b) Fc Domain Modifications Reducing Fc Receptor Binding and/or Effector Function

The Fc domain confers to the (multispecific) antibody favorable pharmacokinetic properties, including a long serum half-life which contributes to good accumulation in the target tissue and a favorable tissue-blood distribution ratio. At the same time it may, however, lead to undesirable targeting of the (multispecific) antibody to cells expressing Fc receptors rather than to the preferred antigen-bearing cells. Moreover, the co-activation of Fc receptor signaling pathways may lead to cytokine release which, in combination with the T cell activating properties and the long half-life of the (multispecific) antibody, results in excessive activation of cytokine receptors and severe side effects upon systemic administration. Activation of (Fc receptor-bearing) immune cells other than T cells may even reduce efficacy of the (multispecific) antibody due to the potential destruction of T cells e.g. by NK cells.

Accordingly, in preferred aspects, the Fc domain of the (multispecific) antibody according to the invention exhibits reduced binding affinity to an Fc receptor and/or reduced effector function, as compared to a native IgG₁ Fc domain. In one such aspect the Fc domain (or the (multispecific) antibody comprising said Fc domain) exhibits less than 50%, particularly less than 20%, more particularly less than 10% and most particularly less than 5% of the binding affinity to an Fc receptor, as compared to a native IgG₁ Fc domain (or a (multispecific) antibody comprising a native IgG₁ Fc domain), and/or less than 50%, particularly less than 20%, more particularly less than 10% and most particularly less than 5% of the effector function, as compared to a native IgG Fc domain domain (or a (multispecific) antibody comprising a native IgG₁ Fc domain). In one aspect, the Fc domain domain (or the (multispecific) antibody comprising said Fc domain) does not substantially bind to an Fc receptor and/or induce effector function. In a preferred aspect the Fc receptor is an Fcγ receptor. In one aspect the Fc receptor is a human Fc receptor. In one aspect the Fc receptor is an activating Fc receptor. In a specific aspect the Fc receptor is an activating human Fcγ receptor, more specifically human FcγRIIIa, FcγRI or FcγRIIa, most specifically human FcγRIIIa. In one aspect the effector function is one or more selected from the group of CDC, ADCC, ADCP, and cytokine secretion. In a preferred aspect, the effector function is ADCC. In one aspect, the Fc domain domain exhibits substantially similar binding affinity to neonatal Fc receptor (FcRn), as compared to a native IgG₁ Fc domain domain. Substantially similar binding to FcRn is achieved when the Fc domain (or the (multispecific) antibody comprising said Fc domain) exhibits greater than about 70%, particularly greater than about 80%, more particularly greater than about 90% of the binding affinity of a native IgG₁ Fc domain (or the (multispecific) antibody comprising a native IgG₁ Fc domain) to FcRn.

In certain aspects the Fc domain is engineered to have reduced binding affinity to an Fc receptor and/or reduced effector function, as compared to a non-engineered Fc domain. In preferred aspects, the Fc domain of the (multispecific) antibody comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In one aspect, the amino acid mutation reduces the binding affinity of the Fc domain to an Fc receptor by at least 2-fold, at least 5-fold, or at least 10-fold. In aspects where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one aspect the (multispecific) antibody comprising an engineered Fc domain exhibits less than 20%, particularly less than 10%, more particularly less than 5% of the binding affinity to an Fc receptor as compared to a (multispecific) antibody comprising a non-engineered Fc domain. In a preferred aspect, the Fc receptor is an Fcγ receptor. In some aspects, the Fc receptor is a human Fc receptor. In some aspects, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the (multispecific) antibody comprising said Fc domain) exhibits greater than about 70% of the binding affinity of a non-engineered form of the Fc domain (or the (multispecific) antibody comprising said non-engineered form of the Fc domain) to FcRn. The Fc domain, or (multispecific) antibodies of the invention comprising said Fc domain, may exhibit greater than about 80% and even greater than about 90% of such affinity. In certain aspects, the Fc domain of the (multispecific) antibody is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one aspect, the reduced effector function is one or more selected from the group of reduced CDC, reduced ADCC, reduced ADCP, and reduced cytokine secretion. In a preferred aspect, the reduced effector function is reduced ADCC. In one aspect the reduced ADCC is less than 20% of the ADCC induced by a non-engineered Fc domain (or a (multispecific) antibody comprising a non-engineered Fc domain).

In one aspect, the amino acid mutation that reduces the binding affinity of the Fc domain to an Fc receptor and/or effector function is an amino acid substitution. In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In preferred aspects, the Fc domain comprises amino acid substitutions at positions P329, L234 and L235 (numberings according to Kabat EU index). In more preferred aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one such aspect, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some aspects, the Fc domain of the (multispecific) antibodies of the invention is an IgG₄ Fc domain, particularly a human IgG₄ Fc domain. In one aspect, the IgG₄ Fc domain comprises an amino acid substitution at position S228, specifically the amino acid substitution S228P (numberings according to Kabat EU index). To further reduce its binding affinity to an Fc receptor and/or its effector function, in one aspect, the IgG₄ Fc domain comprises an amino acid substitution at position L235, specifically the amino acid substitution L235E (numberings according to Kabat EU index). In another aspect, the IgG₄ Fc domain comprises an amino acid substitution at position P329, specifically the amino acid substitution P329G (numberings according to Kabat EU index). In a preferred aspect, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In a preferred aspect, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain aspects, N-glycosylation of the Fc domain has been eliminated. In one such aspect, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or (multispecific) antibodies comprising an Fc domain for Fc receptors may be evaluated using cell lines known to express particular Fc receptors, such as human NK cells expressing FcγIIIa receptor.

Effector function of an Fc domain, or a (multispecific) antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some aspects, binding of the Fc domain to a complement component, specifically to C1q, is reduced. Accordingly, in some aspects wherein the Fc domain is engineered to have reduced effector function, said reduced effector function includes reduced CDC. C1q binding assays may be carried out to determine whether the Fc domain, or the (multispecific) antibody comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18 (12): 1759-1769 (2006); WO 2013/120929).

### B. Polynucleotides

The invention further provides an isolated polynucleotide encoding an antibody of the invention. Said isolated polynucleotide may be a single polynucleotide or a plurality of polynucleotides.

The polynucleotides encoding (multispecific) antibodies of the invention may be expressed as a single polynucleotide that encodes the entire antibody or as multiple (e.g., two or more) polynucleotides that are co-expressed. Polypeptides encoded by polynucleotides that are co-expressed may associate through, e.g., disulfide bonds or other means to form a functional antibody. For example, the light chain portion of an antibody may be encoded by a separate polynucleotide from the portion of the antibody comprising the heavy chain of the antibody. When co-expressed, the heavy chain polypeptides will associate with the light chain polypeptides to form the antibody. In another example, the portion of the antibody comprising one of the two Fc domain subunits and optionally (part of) one or more Fab molecules could be encoded by a separate polynucleotide from the portion of the antibody comprising the other of the two Fc domain subunits and optionally (part of) a Fab molecule. When co-expressed, the Fc domain subunits will associate to form the Fc domain.

In some aspects, the isolated polynucleotide encodes the entire antibody molecule according to the invention as described herein. In other aspects, the isolated polynucleotide encodes a polypeptide comprised in the antibody according to the invention as described herein.

In certain aspects the polynucleotide or nucleic acid is DNA. In other aspects, a polynucleotide of the present invention is RNA, for example, in the form of messenger RNA (mRNA). RNA of the present invention may be single stranded or double stranded.

### C. Recombinant Methods

Antibodies of the invention may be obtained, for example, by solid-state peptide synthesis (e.g. Merrifield solid phase synthesis) or recombinant production. For recombinant production one or more polynucleotide encoding the antibody, e.g., as described above, is isolated and inserted into one or more vectors for further cloning and/or expression in a host cell. Such polynucleotide may be readily isolated and sequenced using conventional procedures. In one aspect a vector, particularly an expression vector, comprising the polynucleotide (i.a. a single polynucleotide or a plurality of polynucleotides) of the invention is provided. Methods which are well known to those skilled in the art can be used to construct expression vectors containing the coding sequence of an antibody along with appropriate transcriptional/translational control signals. These methods include in vitro recombinant DNA techniques, synthetic techniques and in vivo recombination/genetic recombination. Sec, for example, the techniques described in Maniatis et al., Molecular Cloning: A Laboratory Manual, Cold Spring Harbor Laboratory, N.Y. (1989); and Ausubel et al., Current Protocols in Molecular Biology, Greene Publishing Associates and Wiley Interscience, N. Y (1989). The expression vector can be part of a plasmid, virus, or may be a nucleic acid fragment. The expression vector includes an expression cassette into which the polynucleotide encoding the antibody (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the antibody of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible by tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the antibody is desired, DNA encoding a signal sequence may be placed upstream of the nucleic acid encoding an antibody of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain aspects, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the antibody may be included within or at the ends of the antibody (fragment) encoding polynucleotide.

In a further aspect, a host cell comprising a polynucleotide (i.e. a single polynucleotide or a plurality of polynucleotides) of the invention is provided. In certain aspects a host cell comprising a vector of the invention is provided. The polynucleotides and vectors may incorporate any of the features, singly or in combination, described herein in relation to polynucleotides and vectors, respectively. In one such aspect a host cell comprises (e.g. has been transformed or transfected with) one or more vector comprising one or more polynucleotide that encodes (part of) an antibody of the invention. As used herein, the term “host cell” refers to any kind of cellular system which can be engineered to generate the antibody of the invention or fragments thereof. Host cells suitable for replicating and for supporting expression of antibodies are well known in the art. Such cells may be transfected or transduced as appropriate with the particular expression vector and large quantities of vector containing cells can be grown for seeding large scale fermenters to obtain sufficient quantities of the antibody for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one aspect, the host cell is a eukaryotic cell, particularly a mammalian cell, such as a Chinese Hamster Ovary (CHO) cell, a human embryonic kidney (HEK) cell or a lymphoid cell (e.g., YO, NS0, Sp20 cell). In one aspect, the host cell is not a cell within a human body.

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one aspect, a method of producing an antibody according to the invention is provided, wherein the method comprises culturing a host cell comprising a polynucleotide encoding the antibody, as provided herein, under conditions suitable for expression of the antibody, and optionally recovering the antibody from the host cell (or host cell culture medium).

The components of the (multispecific) antibody of the invention may be genetically fused to each other. The (multispecific) antibody can be designed such that its components are fused directly to each other or indirectly through a linker sequence. The composition and length of the linker may be determined in accordance with methods well known in the art and may be tested for efficacy. Examples of linker sequences between different components of (multispecific) antibodies are provided herein. Additional sequences may also be included to incorporate a cleavage site to separate the individual components of the fusion if desired, for example an endopeptidase recognition sequence.

Antibodies prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification, an antibody, ligand, receptor or antigen can be used to which the antibody binds. For example, for affinity chromatography purification of antibodies of the invention, a matrix with protein A or protein G may be used. Sequential Protein A or G affinity chromatography and size exclusion chromatography can be used to isolate an antibody essentially as described in the Examples. The purity of the antibody can be determined by any of a variety of well-known analytical methods including gel electrophoresis, high pressure liquid chromatography, and the like.

### D. Assays

Antibodies provided herein may be identified, screened for, or characterized for their physical/chemical properties and/or biological activities by various assays known in the art.

### 1. Binding Assays

The binding (affinity) of the antibody to an Fc receptor or a target antigen can be determined for example by surface plasmon resonance (SPR), using standard instrumentation such as a BIAcore instrument (Cytiva), and receptors or target proteins such as may be obtained by recombinant expression. Alternatively, binding of antibodies to different receptors or target antigens may be evaluated using cell lines expressing the particular receptor or target antigen, for example by flow cytometry (FACS). A specific illustrative and exemplary aspect for measuring binding activity to CD3 is described in the following.

In one aspect, the binding activity to CD3 is determined by SPR as follows:

SPR is performed on a Biacore T200 instrument (Cytiva) at 25° C. with HBS-P (10 mM HEPES, 150 mM NaCl pH 7.4, 0.05% Surfactant P20) as running and dilution buffer. Biotinylated human CD3ε/δ (a heterodimer of CD3 delta and CD3 epsilon ectodomains fused to a human Fc domain with knob-into-hole modifications and a C-terminal Avi-tag; see SEQ ID NOs 49 and 50) as well as biotinylated anti-huIgG (Capture Select, Thermo Scientific, #7103262100) is immobilized on a Series S Sensor Chip SA (Cytiva, #29104992), resulting in surface densities of at least 1000 resonance units (RU). Anti-CD3 antibodies with a concentration of 2 μg/ml are injected for 30 s at a flow rate of 5 μl/min, and dissociation is monitored for 120 s. The surface is regenerated by injecting 10 mM glycine pH 1.5 for 60 s. Bulk refractive index differences are corrected by subtracting blank injections and by subtracting the response obtained from a blank control flow cell. For evaluation, the binding response 5 seconds after injection end is taken. To normalize the binding signal, the CD3 binding is divided by the anti-huIgG response (the signal (RU) obtained upon capture of the anti-CD3 antibody on the immobilized anti-huIgG antibody). The binding activity to CD3 of an antibody after a certain treatment, relative to the binding activity to CD3 of the antibody after a different treatment is calculated by referencing the binding activity of a sample of the antibody after the certain treatment to the binding activity of a corresponding sample of the antibody after the different treatment.

### 2. Activity Assays

Biological activity of the (multispecific) antibodies of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

### E. Compositions, Formulations, and Routes of Administration

In a further aspect, the invention provides pharmaceutical compositions comprising any of the antibodies provided herein, e.g., for use in any of the below therapeutic methods. In one aspect, a pharmaceutical composition comprises an antibody according to the invention and a pharmaceutically acceptable carrier. In another aspect, a pharmaceutical composition comprises an antibody according to the invention and at least one additional therapeutic agent, e.g., as described below.

Further provided is a method of producing an antibody of the invention in a form suitable for administration in vivo, the method comprising (a) obtaining an antibody according to the invention, and (b) formulating the antibody with at least one pharmaceutically acceptable carrier, whereby a preparation of antibody is formulated for administration in vivo.

Pharmaceutical compositions of the present invention comprise an effective amount of antibody dissolved or dispersed in a pharmaceutically acceptable carrier. The phrase “pharmaceutically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains an antibody and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

An antibody of the invention (and any additional therapeutic agent) can be administered by any suitable means, including parenteral, intrapulmonary, and intranasal, and, if desired for local treatment, intralesional administration. Parenteral administration includes intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the antibodies of the invention may be formulated in aqueous solutions, particularly in physiologically compatible buffers such as Hanks' solution, Ringer's solution, or physiological saline buffer. The solution may contain formulatory agents such as suspending, stabilizing and/or dispersing agents. Alternatively, the antibodies may be in powder form for constitution with a suitable vehicle, e.g., sterile pyrogen-free water, before use. Sterile injectable solutions are prepared by incorporating the antibodies of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Pharmaceutical compositions comprising the antibodies of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The antibodies may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### F. Therapeutic Methods and Compositions

Any of the antibodies provided herein may be used in therapeutic methods. Antibodies of the invention may be used as immunotherapeutic agents, for example in the treatment of cancers.

For use in therapeutic methods, antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, antibodies of the invention for use as a medicament are provided. In further aspects, antibodies of the invention for use in treating a disease are provided. In certain aspects, antibodies of the invention for use in a method of treatment are provided. In one aspect, the invention provides an antibody of the invention for use in the treatment of a disease in an individual in need thereof. In certain aspects, the invention provides an antibody for use in a method of treating an individual having a disease comprising administering to the individual an effective amount of the antibody. In certain aspects the disease to be treated is a proliferative disorder. In certain aspects the disease is cancer, particularly a cancer expressing the second antigen that the antibody of the invention binds to. In certain aspects the method further comprises administering to the individual an effective amount of at least one additional therapeutic agent, e.g., an anti-cancer agent if the disease to be treated is cancer. In further aspects, the invention provides an antibody of the invention for use in inducing lysis of a target cell, particularly a target cell expressing the second antigen that the antibody of the invention binds to. In certain aspects, the invention provides an antibody of the invention for use in a method of inducing lysis of a target cell, particularly a target cell expressing the second antigen that the antibody of the invention binds to, in an individual comprising administering to the individual an effective amount of the antibody to induce lysis of a target cell. An “individual” according to any of the above aspects is a mammal, preferably a human.

In a further aspect, the invention provides for the use of an antibody of the invention in the manufacture or preparation of a medicament. In one aspect the medicament is for the treatment of a disease in an individual in need thereof. In a further aspect, the medicament is for use in a method of treating a disease comprising administering to an individual having the disease an effective amount of the medicament. In certain aspects the disease to be treated is a proliferative disorder. In certain aspects the disease is cancer, particularly a cancer expressing the second antigen that the antibody of the invention binds to. In certain aspects the method further comprises administering to the individual an effective amount of at least one additional therapeutic agent, e.g., an anti-cancer agent if the disease to be treated is cancer. In a further aspect, the medicament is for inducing lysis of a target cell, particularly a target cell expressing the second antigen that the antibody of the invention binds to. In still a further aspect, the medicament is for use in a method of inducing lysis of a target cell, particularly a target cell expressing the second antigen that the antibody of the invention binds to, in an individual comprising administering to the individual an effective amount of the medicament to induce lysis of a target cell. An “individual” according to any of the above aspects may be a mammal, preferably a human.

In a further aspect, the invention provides a medicament (adapted) for the treatment of a disease, comprising the antibody of the invention. In one aspect the medicament is (adapted) for the treatment of a disease in an individual in need thereof. In a further aspect, the medicament is (adapted) for use in a method of treating a disease comprising administering to an individual having the disease an effective amount of the medicament. In certain aspects the disease is a proliferative disorder. In certain aspects the disease is cancer, particularly a cancer expressing the second antigen that the antibody of the invention binds to. In one aspect, the method further comprises administering to the individual an effective amount of at least one additional therapeutic agent, e.g., an anti-cancer agent if the disease to be treated is cancer. In a further aspect, the medicament is for inducing lysis of a target cell, particularly a cancer cell. In still a further aspect, the medicament is for use in a method of inducing lysis of a target cell, particularly a target cell expressing the second antigen that the antibody of the invention binds to, in an individual comprising administering to the individual an effective amount of the medicament to induce lysis of a target cell. An “individual” according to any of the above aspects may be a mammal, preferably a human.

In a further aspect, the invention provides a method for treating a disease. In one aspect, the method comprises administering to an individual having such disease an effective amount of an antibody of the invention. In one aspect a composition is administered to said individual, comprising the antibody of the invention in a pharmaceutically acceptable form. In certain aspects the disease is a proliferative disorder. In certain aspects the disease is cancer, particularly a cancer expressing the second antigen that the antibody of the invention binds to. In certain aspects the method further comprises administering to the individual an effective amount of at least one additional therapeutic agent, e.g., an anti-cancer agent if the disease to be treated is cancer. An “individual” according to any of the above aspects may be a mammal, preferably a human.

In a further aspect, the invention provides a method for inducing lysis of a target cell, particularly a target cell expressing the second antigen that the antibody of the invention binds to. In one aspect the method comprises contacting a target cell with an antibody of the invention in the presence of a T cell, particularly a cytotoxic T cell. In a further aspect, a method for inducing lysis of a target cell, particularly a target cell expressing the second antigen that the antibody of the invention binds to, in an individual is provided. In one such aspect, the method comprises administering to the individual an effective amount of an antibody of the invention to induce lysis of a target cell. In one aspect, an “individual” is a human.

In certain aspects, the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using an antibody of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In one aspect, in particular wherein the antibody is a multispecific antibody binding to HER2 as the second antigen, the cancer is a cancer expressing HER2. In one aspect, in particular wherein the antibody is a multispecific antibody binding to HER2 as the second antigen, the cancer is a cancer selected from the group consisting of breast cancer, gastrointestinal cancer, and lung cancer.

A skilled artisan readily recognizes that in many cases the antibody may not provide a cure but may only provide partial benefit. In some aspects, a physiological change having some benefit is also considered therapeutically beneficial. Thus, in some aspects, an amount of antibody that provides a physiological change is considered an “effective amount”. The subject, patient, or individual in need of treatment is typically a mammal, more specifically a human.

In some aspects, an effective amount of an antibody of the invention is administered to an individual for the treatment of disease.

For the prevention or treatment of disease, the appropriate dosage of an antibody of the invention (when used alone or in combination with one or more other additional therapeutic agents) will depend on the type of disease to be treated, the route of administration, the body weight of the patient, the type of antibody, the severity and course of the disease, whether the antibody is administered for preventive or therapeutic purposes, previous or concurrent therapeutic interventions, the patient's clinical history and response to the antibody, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. The progress of this therapy is easily monitored by conventional techniques and assays.

The antibodies of the invention will generally be used in an amount effective to achieve the intended purpose. For use to treat or prevent a disease condition, the antibodies of the invention, or pharmaceutical compositions thereof, are administered or applied in an effective amount.

For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art.

Dosage amount and interval may be adjusted individually to provide plasma levels of the antibodies which are sufficient to maintain therapeutic effect. Therapeutically effective plasma levels may be achieved by administering multiple doses each day. Levels in plasma may be measured, for example, by HPLC.

An effective dose of the antibodies of the invention will generally provide therapeutic benefit without causing substantial toxicity. Toxicity and therapeutic efficacy of an antibody can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Antibodies that exhibit large therapeutic indices are preferred. In one aspect, the antibody according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with antibodies of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The antibodies of the invention may be administered in combination with one or more other agents in therapy. For instance, an antibody of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular disease being treated, preferably those with complementary activities that do not adversely affect each other.

In certain aspects, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers.

In some aspects, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of antibody used, the type of disorder or treatment, and other factors discussed above. The antibodies are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the antibody of the invention can occur prior to, simultaneously, and/or following, administration of the additional therapeutic agent and/or adjuvant. Antibodies of the invention may also be used in combination with radiation therapy.

### G. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### H. Methods and Compositions for Diagnostics and Detection

In certain aspects, any of the antibodies provided herein is useful for detecting the presence of its target (e.g. HER2) in a biological sample. The term “detecting” as used herein encompasses quantitative or qualitative detection. In certain aspects, a biological sample comprises a cell or tissue, such as tumour tissue.

In one aspect, an antibody according to the invention for use in a method of diagnosis or detection is provided. In a further aspect, a method of detecting the presence of CD3 or HER2 in a biological sample is provided. In certain aspects, the method comprises contacting the biological sample with an antibody of the present invention under conditions permissive for binding of the antibody to CD3 or HER2, and detecting whether a complex is formed between the antibody and CD3 or HER2. Such method may be an in vitro or in vivo method. In one aspect, an antibody of the invention is used to select subjects eligible for therapy with an antibody that binds CD3 and/or HER2, e.g. where CD3 and/or HER2 is a biomarker for selection of patients.

Exemplary disorders that may be diagnosed using an antibody of the invention include cancer, particularly breast cancer.

In certain aspects, an antibody according to the present invention is provided, wherein the antibody is labelled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32p, 14C, 1251, 3H, and 1311, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

III. SEQUENCES

| | | SEQ ID |
|---|---|---|
| | Amino Acid Sequence | NO |
| CD3 HCDR1 | SYAMN | 1 |
| (CD3_{orig}, | | |
| P1AI7912, | | |
| P1AI7913, | | |
| P1AI7915) | | |
| CD3 HCDR2 | RIRSKYNNYATYYADSVKG | 2 |
| (CD3orig) | | |
| CD3 HCDR3 | ASNFPASYVSYFAY | 3 |
| (CD3_{orig}, | | |
| P1AI7912, | | |
| P1AI7913, | | |
| P1AI7915) | | |
| CD3 VH | EVQLLESGGGLVQPGGSLRLSCAASGFTFSSYAMNWVRQAPGKGLEWVSRIRSKYN | 4 |
| (CD3_{orig}) | NYATYYADSVKGRFTISRDDSKNTLYLQMNSLRAEDTAVYYCVRASNFPASYVSYF | |
| | AYWGQGTLVTVSS | |
| CD3 LCDR1 | GSSTGAVTTSNYAN | 5 |
| (CD3_{orig}, | | |
| P1AI7912, | | |
| P1AI7913, | | |
| P1AI7915) | | |
| CD3 LCDR2 | GTNKRAP | 6 |
| (CD3_{orig}) | | |
| CD3 LCDR3 | ALWYSNLWV | 7 |
| (CD3_{orig}, | | |
| P1AI7912, | | |
| P1AI7913, | | |
| P1AI7915) | | |
| CD3 VL | QAVVTQEPSLTVSPGGTVTLTCGSSTGAVTTSNYANWVQEKPGQAFRGLIGGINKR | 8 |
| (CD3_{orig}) | APGTPARFSGSLLGGKAALTLSGAQPEDEAEYYCALWYSNLWVFGGGTKLTVL | |
| CD3 HCDR2 | RVRTTYNNREPYYADSVKG | 9 |
| (P1AI7912) | | |
| CD3 VH | EVQLLESGGGLVQPGGSLRLSCAASGFTFSSYAMNWVRQAPGKGLEWVGRVRTTYN | 10 |
| (P1AI7912) | NREPYYADSVKGRFTISRDDSKNTLYLQMNSLRAEDTAVYYCVRASNFPASYVSYF | |
| | AYWGQGTLVTVSS | |
| CD3 LCDR2 | GTNKLAP | 11 |
| (P1AI7912, | | |
| P1AI7913, | | |
| P1AI7915) | | |
| CD3 VL | QAVVTQEPSLTVSPGGTVTLTCGSSTGAVTTSNYANWVQEKPGQPPRGLIGGINKL | 12 |
| (P1AI7912, | APGTPARFSGSLLGGKAALTLSGAQPEDEAEYYCALWYSNLWVFGGGTKLTVL | |
| P1AI7913, | | |
| P1AI7915) | | |
| CD3 HCDR2 | RVRTKYNNREPYYADSVKG | 13 |
| (P1AI7913) | | |
| CD3 VH | EVQLLESGGGLVQPGGSLRLSCAASGFTFSSYAMNWVRQAPGKGLEWVGRVRTKYN | 14 |
| (P1AI7913) | NREPYYADSVKGRFTISRDDSKNTLYLQMNSLRAEDTAVYYCVRASNFPASYVSYF | |
| | AYWGQGTLVTVSS | |
| CD3 HCDR2 | RQRTQYNNREDYYADSVKG | 15 |
| (P1AI7915) | | |
| CD3 VH | EVQLLESGGGLVQPGGSLRLSCAASGFTFSSYAMNWVRQAPGKGLEWVGRQRTQYN | 16 |
| (P1AI7915) | NREDYYADSVKGRFTISRDDSKNTLYLQMNSLRAEDTAVYYCVRASNFPASYVSYF | |
| | AYWGQGTLVTVSS | |
| CD3 VH | EVQLLESGGGLVQPGGSLRLSCAASGFTFSSYAMNWVRQAPGKGLEWVGRVRTTYN | 17 |
| (P1AI7912) | NREPYYADSVKGRFTISRDDSKNTLYLQMNSLRAEDTAVYYCVRASNFPASYVSYF | |
| L108S | AYWGQGTSVTVSG | |
| /S113G | | |
| CD3 VH | EVQLLESGGGLVQPGGSLRLSCAASGFTFSSYAMNWVRQAPGKGLEWVGRVRTKYN | 18 |
| (P1AI7913) | NREPYYADSVKGRFTISRDDSKNTLYLQMNSLRAEDTAVYYCVRASNFPASYVSYF | |
| L108S | AYWGQGTSVTVSG | |
| /S113G | | |
| CD3 VH | EVQLLESGGGLVQPGGSLRLSCAASGFTFSSYAMNWVRQAPGKGLEWVGRQRTQYN | 19 |
| (P1AI7915) | NREDYYADSVKGRFTISRDDSKNTLYLQMNSLRAEDTAVYYCVRASNFPASYVSYF | |
| L108S | AYWGQGTSVTVSG | |
| /S113G | | |
| HER2 | DTYIH | 20 |
| HCDR1 | | |
| HER2 | RIYPTNGYTRYADSVKG | 21 |
| HCDR2 | | |
| HER2 | WGGEGFYAMDY | 22 |
| HCDR3 | | |
| HER2 VH | EVQLVESGGGLVQPGGSLRLSCAASGFNIKDTYIHWVRQAPGKGLEWVARIYPING | 23 |
| | YTRYADSVKGRFTISADTSKNTAYLQMNSLRAEDTAVYYCSRWGGEGFYAMDYWGQ | |
| | GTLVTVSS | |
| HER2 | RASQDVNTAVA | 24 |
| LCDR1 | | |
| HER2 | SASFLYS | 25 |
| LCDR2 | | |
| HER2 | QQHYTTPPT | 26 |
| LCDR3 | | |
| HER2 VL | DIQMTQSPSSLSASVGDRVTITCRASQDVNTAVAWYQQKPGKAPKLLIYSASFLYS | 27 |
| | GVPSRFSGSRSGTDFTLTISSLQPEDFATYYCQQHYTTPPTFGQGTKVEIK | |
| HER2 VH- | EVQLVESGGGLVQPGGSLRLSCAASGFNIKDTYIHWVRQAPGKGLEWVARIYPING | 28 |
| CH1-CD3 | YTRYADSVKGRFTISADTSKNTAYLQMNSLRAEDTAVYYCSRWGGEGFYAMDYWGQ | |
| (CD3_{orig}) VL- | GTLVTVSSASTKGPSVFPLAPSSKSTSGGTAALGCLVEDYFPEPVTVSWNSGALTS | |
| CH1-Fc | GVHTFPAVLQSSGLYSLSSVVTVPSSSLGTQTYICNVNHKPSNTKVDEKVEPKSCG | |
| (knob, | GGGSGGGGQAVVTQEPSLTVSPGGTVTLTCGSSTGAVTTSNYANWVQEKPGQAFRG | |
| PGLALA) | LIGGTNKRAPGTPARFSGSLLGGKAALTLSGAQPEDEAEYYCALWYSNLWVFGGGT | |
| | KLTVLSSASTKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSCDK | |
| | THTCPPCPAPEAAGGPSVFLFPPKPKDTLMISRTPEVTCVVVDVSHEDPEVKENWY | |
| | VDGVEVHNAKTKPREEQYNSTYRVVSVLTVLHQDWLNGKEYKCKVSNKALGAPIEK | |
| | TISKAKGQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKSLSLSP | |
| HER2 VH- | EVQLVESGGGLVQPGGSLRLSCAASGFNIKDTYIHWVRQAPGKGLEWVARIYPING | 29 |
| CH1-Fc | YTRYADSVKGRFTISADTSKNTAYLQMNSLRAEDTAVYYCSRWGGEGFYAMDYWGQ | |
| (hole, | GTLVTVSSASTKGPSVFPLAPSSKSTSGGTAALGCLVEDYFPEPVTVSWNSGALTS | |
| PGLALA) | GVHTFPAVLQSSGLYSLSSVVTVPSSSLGTQTYICNVNHKPSNTKVDEKVEPKSCD | |
| | KTHTCPPCPAPEAAGGPSVFLFPPKPKDTLMISRTPEVTCVVVDVSHEDPEVKENW | |
| | YVDGVEVHNAKTKPREEQYNSTYRVVSVLTVLHQDWLNGKEYKCKVSNKALGAPIE | |
| | KTISKAKGQPREPQVCTLPPSRDELTKNQVSLSCAVKGFYPSDIAVEWESNGQPEN | |
| | NYKTTPPVLDSDGSFFLVSKLTVDKSRWQQGNVFSCSVMHEALHNHYTQKSLSLSP | |
| CD3 (CD3_{orig}) | EVQLLESGGGLVQPGGSLRLSCAASGFTFSSYAMNWVRQAPGKGLEWVSRIRSKYN | 30 |
| VH-CL | NYATYYADSVKGRFTISRDDSKNTLYLQMNSLRAEDTAVYYCVRASNFPASYVSYF | |
| | AYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| HER2 VL-CL | DIQMTQSPSSLSASVGDRVTITCRASQDVNTAVAWYQQKPGKAPKLLIYSASFLYS | 31 |
| | GVPSRFSGSRSGTDFTLTISSLQPEDFATYYCQQHYTTPPTFGQGTKVEIKRTVAA | |
| | PSVFIFPPSDRKLKSGTASVVCLLNNFYPREAKVQWKVDNALQSGNSQESVTEQDS | |
| | KDSTYSLSSTLTLSKADYEKHKVYACEVTHQGLSSPVTKSENRGEC | |
| CD3 | EVQLLESGGGLVQPGGSLRLSCAASGFTFSSYAMNWVRQAPGKGLEWVGRVRTTYN | 32 |
| (P1AI7912) | NREPYYADSVKGRFTISRDDSKNTLYLQMNSLRAEDTAVYYCVRASNFPASYVSYF | |
| VH-CL | AYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| CD3 | EVQLLESGGGLVQPGGSLRLSCAASGFTFSSYAMNWVRQAPGKGLEWVGRVRTKYN | 33 |
| (P1AI7913) | NREPYYADSVKGRFTISRDDSKNTLYLQMNSLRAEDTAVYYCVRASNFPASYVSYF | |
| VH-CL | AYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| CD3 | EVQLLESGGGLVQPGGSLRLSCAASGFTFSSYAMNWVRQAPGKGLEWVGRQRTQYN | 34 |
| (P1AI7915) | NREDYYADSVKGRFTISRDDSKNTLYLQMNSLRAEDTAVYYCVRASNFPASYVSYF | |
| VH-CL | AYWGQGTLVTVSSASVAAPSVFIFPPSDEQLKSGTASVVCLLNNFYPREAKVQWKV | |
| | DNALQSGNSQESVTEQDSKDSTYSLSSTLTLSKADYEKHKVYACEVTHQGLSSPVT | |
| | KSFNRGEC | |
| CD3 | EVQLLESGGGLVQPGGSLRLSCAASGFTFSSYAMNWVRQAPGKGLEWVGRQRTQYN | 35 |
| (P1AI7915) | NREDYYADSVKGRFTISRDDSKNTLYLQMNSLRAEDTAVYYCVRASNFPASYVSYF | |
| VH-CL | AYWGQGTSVTVSGASVAAPSVFIFPPSDEQLKSGTASVVCLLNNFYPREAKVQWKV | |
| L108S/ | DNALQSGNSQESVTEQDSKDSTYSLSSTLTLSKADYEKHKVYACEVTHQGLSSPVT | |
| S113G | KSFNRGEC | |
| Human CD3 | QDGNEEMGGITQTPYKVSISGTTVILTCPQYPGSEILWQHNDKNIGGDEDDKNIGS | 36 |
| | DEDHLSLKEFSELEQSGYYVCYPRGSKPEDANFYLYLRARVCENCMEMDVMSVATI | |
| | VIVDICITGGLLLLVYYWSKNRKAKAKPVTRGAGAGGRQRGQNKERPPPVPNPDYE | |
| | PIRKGQRDLYSGLNQRRI | |
| Cynomolgus | QDGNEEMGSITQTPYQVSISGTTVILTCSQHLGSEAQWQHNGKNKEDSGDRLFLPE | 37 |
| CD3 | FSEMEQSGYYVCYPRGSNPEDASHHLYLKARVCENCMEMDVMAVATIVIVDICITL | |
| | GLLLLVYYWSKNRKAKAKPVTRGAGAGGRQRGQNKERPPPVPNPDYEPIRKGQQDL | |
| | YSGLNQRRI | |
| hIgG₁ Fc | DKTHTCPPCPAPELLGGPSVFLFPPKPKDTLMISRTPEVTCVVVDVSHEDPEVKEN | 38 |
| region | WYVDGVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | P | |
| Human kappa | RTVAAPSVFIFPPSDEQLKSGTASVVCLLNNFYPREAKVQWKVDNALQSGNSQESV | 39 |
| CL domain | TEQDSKDSTYSLSSTLTLSKADYEKHKVYACEVTHQGLSSPVTKSENRGEC | |
| Human | QPKAAPSVTLFPPSSEELQANKATLVCLISDFYPGAVTVAWKADSSPVKAGVETTT | 40 |
| lambda CL | PSKQSNNKYAASSYLSLTPEQWKSHRSYSCQVTHEGSTVEKTVAPTECS | |
| domain | | |
| Human IgG₁ | ASTKGPSVFPLAPSSKSTSGGTAALGCLVKDYFPEPVTVSWNSGALTSGVHTFPAV | 41 |
| heavy chain | LQSSGLYSLSSVVTVPSSSLGTQTYICNVNHKPSNTKVDKKVEPKSCDKTHTCPPC | |
| constant | PAPELLGGPSVFLFPPKPKDTLMISRTPEVTCVVVDVSHEDPEVKFNWYVDGVEVH | |
| region (CH1- | NAKTKPREEQYNSTYRVVSVLTVLHQDWLNGKEYKCKVSNKALPAPIEKTISKAKG | |
| CH2-CH3) | QPREPQVYTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSP | |
| Linker | GGGGSGGGGS | 42 |
| Linker | DGGGGSGGGGS | 43 |
| Linker | GGGGSGGGGG | 44 |
| Linker | DGGGGSGGGGG | 45 |
| HER2 VH- | EVQLVESGGGLVQPGGSLRLSCAASGFNIKDTYIHWVRQAPGKGLEWVARIYPING | 46 |
| CH1-CD3 | YTRYADSVKGRFTISADTSKNTAYLQMNSLRAEDTAVYYCSRWGGEGFYAMDYWGQ | |
| (P1AI7912, | GTLVTVSSASTKGPSVFPLAPSSKSTSGGTAALGCLVEDYFPEPVTVSWNSGALTS | |
| P1AI7913, | GVHTFPAVLQSSGLYSLSSVVTVPSSSLGTQTYICNVNHKPSNTKVDEKVEPKSCG | |
| P1AI7915) | GGGSGGGGQAVVTQEPSLTVSPGGTVTLTCGSSTGAVTTSNYANWVQEKPGQPPRG | |
| VL-CH1-Fc | LIGGTNKLAPGTPARFSGSLLGGKAALTLSGAQPEDEAEYYCALWYSNLWVFGGGT | |
| (knob, | KLTVLSSASTKGPSVFPLAPSSKSTSGGTAALGCLVKDYFPEPVTVSWNSGALTSG | |
| PGLALA) | VHTFPAVLQSSGLYSLSSVVTVPSSSLGTQTYICNVNHKPSNTKVDKKVEPKSCDK | |
| | THTCPPCPAPEAAGGPSVFLFPPKPKDTLMISRTPEVTCVVVDVSHEDPEVKENWY | |
| | VDGVEVHNAKTKPREEQYNSTYRVVSVLTVLHQDWLNGKEYKCKVSNKALGAPIEK | |
| | TISKAKGQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKSLSLSP | |
| HER2 VH- | EVQLVESGGGLVQPGGSLRLSCAASGFNIKDTYIHWVRQAPGKGLEWVARIYPING | 47 |
| CH1-CD3 | YTRYADSVKGRFTISADTSKNTAYLQMNSLRAEDTAVYYCSRWGGEGFYAMDYWGQ | |
| (P1AI7915) | GTLVTVSSASTKGPSVFPLAPSSKSTSGGTAALGCLVEDYFPEPVTVSWNSGALTS | |
| VL-CH1-Fc | GVHTFPAVLQSSGLYSLSSVVTVPSSSLGTQTYICNVNHKPSNTKVDEKVEPKSCG | |
| (knob, | GGGSGGGGQAVVTQEPSLTVSPGGTVTLTCGSSTGAVTTSNYANWVQEKPGQPPRG | |
| PGLALA) | LIGGTNKLAPGTPARFSGSLLGGKAALTLSGAQPEDEAEYYCALWYSNLWVFGGGT | |
| “SEAEK” | KLTVLSEAEKKGPSVFPLAPSSKSTSGGTAALGCLVKDYFPEPVTVSWNSGALTSG | |
| | VHTFPAVLQSSGLYSLSSVVTVPSSSLGTQTYICNVNHKPSNTKVDKKVEPKSCDK | |
| | THTCPPCPAPEAAGGPSVFLFPPKPKDTLMISRTPEVTCVVVDVSHEDPEVKENWY | |
| | VDGVEVHNAKTKPREEQYNSTYRVVSVLTVLHQDWLNGKEYKCKVSNKALGAPIEK | |
| | TISKAKGQPREPQVYTLPPCRDELTKNQVSLWCLVKGFYPSDIAVEWESNGQPENN | |
| | YKTTPPVLDSDGSFFLYSKLTVDKSRWQQGNVFSCSVMHEALHNHYTQKSLSLSP | |
| Linker | GGGGSGGGG | 48 |
| Human CD3 | QDGNEEMGGITQTPYKVSISGTTVILTCPQYPGSEILWQHNDKNIGGDEDDKNIGS | 49 |
| epsilon stalk- | DEDHLSLKEFSELEQSGYYVCYPRGSKPEDANFYLYLRARVSENCVDEQLYFQGGS | |
| Fc(knob)- | PKSADKTHTCPPCPAPELLGGPSVFLFPPKPKDTLMISRTPEVTCVVVDVSHEDPE | |
| Avi | VKFNWYVDGVEVHNAKTKPREEQYNSTYRVVSVLTVLHQDWLNGKEYKCKVSNKAL | |
| | PAPIEKTISKAKGQPREPQVYTLPPCRDELTKNQVSLWCLVKGFYPSDIAVEWESN | |
| | GQPENNYKTTPPVLDSDGSFFLYSKLTVDKSRWQQGNVFSCSVMHEALHNHYTQKS | |
| | LSLSPGKSGGLNDIFEAQKIEWHE | |
| Human CD3 | FKIPIEELEDRVFVNCNTSITWVEGTVGTLLSDITRLDLGKRILDPRGIYRCNGTD | 50 |
| delta stalk- | IYKDKESTVQVHYRMCRSEQLYFQGDKTHTCPPCPAPELLGGPSVFLFPPKPKDTL | |
| Fc (hole)- | MISRTPEVTCVVVDVSHEDPEVKFNWYVDGVEVHNAKTKPREEQYNSTYRVVSVLT | |
| Avi | VLHQDWLNGKEYKCKVSNKALPAPIEKTISKAKGQPREPQVCTLPPSRDELTKNQV | |
| | SLSCAVKGFYPSDIAVEWESNGQPENNYKTTPPVLDSDGSFFLVSKLTVDKSRWQQ | |
| | GNVFSCSVMHEALHNHYTQKSLSLSPGKSGGLNDIFEAQKIEWHE | |
| Cynomolgus | QDGNEEMGSITQTPYQVSISGTTVILTCSQHLGSEAQWQHNGKNKEDSGDRLFLPE | 51 |
| CD3 epsilon | FSEMEQSGYYVCYPRGSNPEDASHHLYLKARVSENCVDEQLYFQGGSPKSADKTHT | |
| stalk-Fc | CPPCPAPELLGGPSVFLFPPKPKDTLMISRTPEVTCVVVDVSHEDPEVKFNWYVDG | |
| (knob)-Avi | VEVHNAKTKPREEQYNSTYRVVSVLTVLHQDWLNGKEYKCKVSNKALPAPIEKTIS | |
| | KAKGQPREPQVYTLPPCRDELTKNQVSLWCLVKGFYPSDIAVEWESNGQPENNYKT | |
| | TPPVLDSDGSFFLYSKLTVDKSRWQQGNVFSCSVMHEALHNHYTQKSLSLSPGKSG | |
| | GLNDIFEAQKIEWHE | |
| Cynomolgus | FKIPVEELEDRVFVKCNTSVTWVEGTVGILLINNTRLDLGKRILDPRGIYRCNGTD | 52 |
| CD3 delta | IYKDKESAVQVHYRMSQNCVDEQLYFQGGSPKSADKTHTCPPCPAPELLGGPSVFL | |
| stalk-Fc | FPPKPKDTLMISRTPEVTCVVVDVSHEDPEVKFNWYVDGVEVHNAKTKPREEQYNS | |
| (hole)-Avi | TYRVVSVLTVLHQDWLNGKEYKCKVSNKALPAPIEKTISKAKGQPREPQVCTLPPS | |
| | RDELTKNQVSLSCAVKGFYPSDIAVEWESNGQPENNYKTTPPVLDSDGSFFLVSKL | |
| | TVDKSRWQQGNVFSCSVMHEALHNHYTQKSLSLSPGKSGGLNDIFEAQKIEWHE | |

### IV. EXAMPLES

The following are examples of methods and compositions of the invention. It is understood that various other aspects may be practiced, given the general description provided above.

### Example 1-Preparation of Optimized Anti-CD3 (Multispecific) Antibodies

For the generation of an optimized CD3 binder variant, we used structure-based calculations to identify positively-charged and hydrophobic patches (MOE, Discovery Studio) and sequence-based predictions (NetMHCIIpan 4.0) to predict potential T-cell epitopes. Besides the parental binder CD3_{orig}, we analyzed the data of more than 1000 different CD3 binder variants tested before. For the subsequent design rounds, the approach revolved around substituting solvent-exposed and non-antigen binding variable region amino acids containing positively charged or hydrophobic side chains by residues with preferably negatively-charged or polar side chains, while constraining the sequence space such that the number of predicted MHC-II binding peptides in the designed sequences was minimized.

We used PIAH4929 (termed CD3_{orig} herein, and comprising the VH and VL sequences of SEQ ID NOs 4 and 8, respectively) as a parental CD3 binder backbone (see WO 2021/255142, incorporated herein by reference in its entirety).

We focused on phenotypes with beneficial specificity, reduced immunogenicity risk, reduced hydrophobic or positive patchiness, and moderate affinity, while maintaining other desirable features such as stability, humanness etc. Our goal in engineering increased specificity was, besides other considerations, the reduction of unwanted off-target side-effects. By reducing positive patchiness, we aimed for reduced nonspecific uptake/presentation of the antibody in addition to potentially increased half-life. The reduction of strong potential T-cell epitopes was done to yield less recognition of MHC II presented peptides by T-cells and potentially immunogenic reactions caused by the antibody which might result in anti-drug antibody (ADA) formation in patients. By reducing affinity of CD3_{orig}, we aimed to reach a sweet spot of potency and cytokine release. It was recently described that by using CD38 antibodies with reduced CD38 affinity, T cell killing can be uncoupled from cytokine secretion, resulting in superior safety and prevention or reduction of cytokine release. (see e.g. Staflin et al. JCI Insight 5, doi: 10.1172/jci.insight.133757 (2020)).

We underwent three consecutive design/make/test cycles to generate a large number of >100 one-armed IgG anti-CD3 antibodies by combinatorial protein engineering methods known in the art. The clones were subjected to a screening cascade for productivity, purity, and binding. Interesting clones were analyzed further by biochemical and in vitro methods. After two initial screening rounds with >100 variants, from a pool of 35 third-round molecules, we selected three that fulfilled our criteria best.

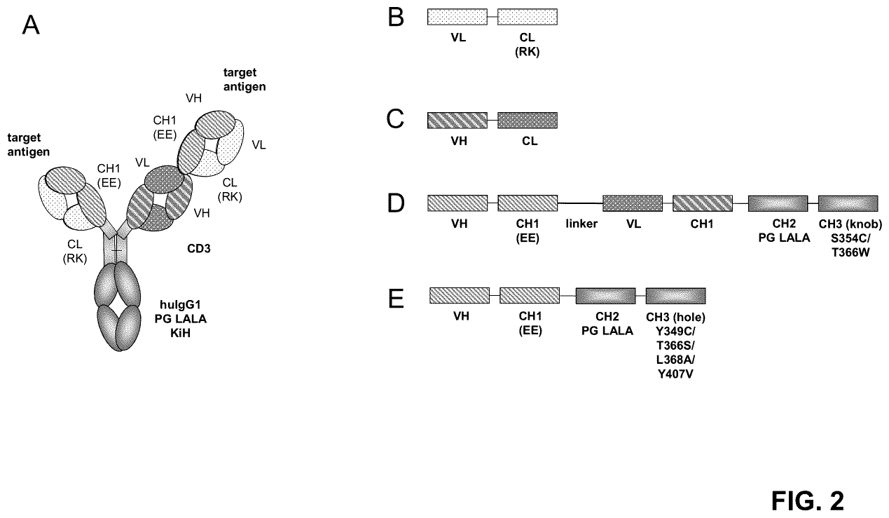

For direct comparison, all binders were produced as 2+1 IgG Crossfab “inverted” T-cell bispecific antibody (TCB) molecules, as depicted in FIG. 2A, using a HER2 antibody as an exemplary target cell antigen binding domain (SEQ ID NOs 20-27). The variable regions of heavy and light chain DNA sequences were subcloned in frame with either the constant heavy chain or the constant light chain pre-inserted into the respective recipient mammalian expression vectors as shown in FIGS. 2B-2E.

Sequences of the optimized anti-CD3 antibodies are given in the SEQ ID NOS indicated in Table 1.

TABLE 1
Sequences of optimized anti-CD3 antibodies generated in the present Examples.

| | | | | VH | | | | |
|---|---|---|---|---|---|---|---|---|
| | HCDR1 | HCDR2 | HCDR3 | (SEQ | LCDR1 | LCDR2 | LCDR3 | |
| | (SEQ ID | (SEQ ID | (SEQ ID | ID | (SEQ ID | (SEQ ID | (SEQ ID | VL |
| | NO) | NO) | NO) | NO) | NO) | NO) | NO) | (SEQ ID NO) |
| P1AI7912 | 1 | 9 | 3 | 10 | 5 | 11 | 7 | 12 |
| | | | | 17* | | | | |
| P1AI7913 | 1 | 13 | 3 | 14 | 5 | 11 | 7 | 12 |
| | | | | 18* | | | | |
| P1AI7915 | 1 | 15 | 3 | 16 | 5 | 11 | 7 | 12 |
| | | | | 19* | | | | |
| P1AH4929 | 1 | 2 | 3 | 4 | 5 | 6 | 7 | 8 |
| CD3_{orig} | | | | | | | | |

*with “crossover mutations”: L108S and S113G (Kabat numbering) (these mutations are predicted to reduce immunogenicity of the binder if placed in a VH/VL crossover Fab molecule)

To improve correct pairing of the light chains with the corresponding heavy chains, mutations were introduced in the human CL (E123R, Q124K) and the human CH1 (K147E, K213E) of the HER2 binding Fab molecule.

For correct pairing of the heavy chains (formation of a heterodimeric molecule), knob-into-hole mutations were introduced in the constant region of the antibody heavy chains (T366W/S354C and T366S/L368A/Y407V/Y349C, respectively).

Furthermore, the P329G, L234A and L235A mutations were introduced in the constant region of the antibody heavy chains to abrogate binding to Fcγ receptors.

Full sequences of the prepared TCB molecules are given in the SEQ ID NOs indicated in Table 2. A corresponding molecule comprising CD3_{orig} as CD3 binder was also prepared.

TABLE 2
Sequences of TCB molecules generated in the present Examples.

| | HC1 | HC2 | LC1 | LC2 |
|---|---|---|---|---|
| | (knob) | (hole) | (CD3) | (HER2) |
| | (SEQ | (SEQ | (SEQ | (SEQ |
| Binder | ID NO) | ID NO) | ID NO) | ID NO) |
| P1AI7912 | 46 | 29 | 32 | 31 |
| P1AI7913 | 46 | 29 | 33 | 31 |
| P1AI7915 | 46 | 29 | 34 | 31 |
| P1AI7915* | 47 | 29 | 35 | 31 |
| P1AH4929 | 28 | 29 | 30 | 31 |
| CD3_{orig} | | | | |

*with “crossover mutations”: L108S and S113G (Kabat numbering) in the CD3 VH and the amino acid sequence SEAEK immediately adjacent to the C-terminus of the CD3 VL (these mutations are predicted to reduce immunogenicity of a TCB molecule with a VH/VL crossover Fab molecule)

Antibodies and bispecific antibodies were generated by transient transfection of Expi293F cells. Cells were seeded in Expi293 media (Gibco, #1435101) at a density of 2.5×10⁶/ml. Expression vectors and ExpiFectamine (Gibco, ExpiFectamine transfection kit, #13385544) were separately mixed in OptiMEM (Gibco, #11520386). After 5 minutes both solutions were combined, mixed by pipetting and incubated for 15-20 minutes at room temperature. Cells were added to the vector/ExpiFectamine solution and incubated for 24 hours at 37° C. in a shaking incubator with a 5% CO₂ atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below. Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (#UFC903096), and aggregated protein was separated from monomeric protein by size exclusion chromatography in 20 mM histidine, 140 mM sodium chloride, pH 6.0.

The concentrations of purified proteins were determined by measuring the absorption at 280 nm using the mass extinction coefficient calculated on the basis of the amino acid sequence according to Pace et al., Protein Science 4, 2411-1423 (1995). Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K₂HPO₄, 125 mM NaCl, 200 mM L-arginine monohydrocloride, pH 6.7 or 200 mM KH₂PO₄, 250 mM KCl pH 6.2, respectively).

Results from the biochemical and biophysical analysis of the prepared TCB molecules are given in Table 3. All TCB molecules could be produced in good quality.

TABLE 3
Biochemical and biophysical analysis of anti-CD3
antibodies in TCB format.

| | | analytical size exclusion | | | CE-SDS |
|---|---|---|---|---|---|
| | | chromatography | | | (main |
| anti-CD3 | yield | [%] | | | peak) |
| antibody | [mg/l] | HMW | monomer | LMW | [%] |
| P1AI7912 | 40.7 | 0.7 | 96.1 | 3.2 | 97.1 |
| P1AI7913 | 34.3 | 1.2 | 96 | 2.8 | 98.0 |
| P1AI7915 | 33.5 | 0.4 | 98.5 | 1.2 | 90.7 |
| P1AH4929 | 10.3 | 0 | 99 | 1 | 98.37 |
| CD3_{orig} | | | | | |

### Example 2-Determination of Thermal Stability of Optimized Anti-CD3 (Multispecific) Antibodies

Thermal stability of the anti-CD3 antibodies prepared in Example 1 (in TCB format) was monitored by Dynamic Light Scattering (DLS) and by monitoring of temperature dependent intrinsic protein fluorescence by applying a temperature ramp using an Optim 2 instrument (Avacta Analytical, UK).

10 μg of filtered protein sample with a protein concentration of 1 mg/ml was applied in duplicate to the Optim 2. The temperature was ramped from 25 to 85° C. at 0.1° C./min, with the ratio of fluorescence intensity at 350 nm/330 nm and scattering intensity at 266 nm being collected.

The results are shown in Table 4. The midpoint of the observed temperature induced unfolding transition (Tₘ) of all the optimized CD3 binders produced in Example 1 is comparable to CD3_{orig}.

TABLE 4
Thermal stability of anti-CD3 antibodies in TCB format
as measured by dynamic light scattering and change of
temperature dependent intrinsic protein fluorescence.

| anti-CD3 antibody | Tₘ [° C.] |
|---|---|
| P1AI7912 | 65 |
| P1AI7913 | 65 |
| P1AI7915 | 65 |
| P1AH4929 CD3_{orig} | 66 |

### Example 3-Functional Characterization of Optimized Anti-CD3 (Multispecific) Antibodies by Surface Plasmon Resonance (SPR)

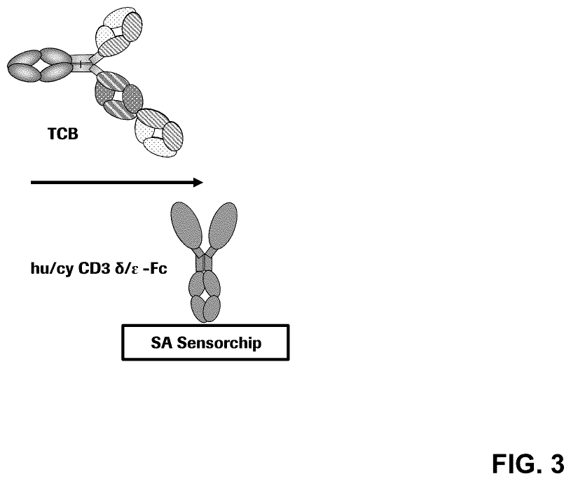

CD3 binding was investigated using surface plasmon resonance (SPR) on a Biacore T200 instrument (Cytiva). All experiments were performed at 25° C. using HEPES buffered saline (20 mM HEPES, 140 mM NaCl, 0.05% PS-20) pH 7.4. The experimental setup is schematically depicted in FIG. 3.

Biotinylated human and cynomolgus CD3ε stalk-Fc (knob)-Avi/CD38 stalk-Fc (hole) (CD3E/8, see SEQ ID NOs 49 and 50 (human) and SEQ ID NOs 51 and 52 (cynomolgus)) were coupled to a Series S Sensor Chip SA (Cytiva, #29104992) according to the manufacturer's instructions. A concentration series ranging from 0.37 to 300 nM (1:3) of the TCB antibody molecules were injected with 90 s association time, followed by a buffer wash of 300 s for monitoring the dissociation phase. Bulk refractive index differences were corrected by subtracting the response obtained from a mock surface. Blank injections were subtracted (double referencing). Kinetic constants were derived using the Biacore T200 Evaluation Software (Cytiva), to fit rate equations for 1:1 Langmuir binding by numerical integration. The half-life (t_{1/2}) of the interaction was calculated using the formula t_{1/2}=ln2/k_{off}.

In Table 5 all kinetic parameters of the binding of the optimized anti-CD3 antibodies compared to the previously described binder CD3_{orig} are listed. The optimized anti-CD3 antibodies (in TCB format) are binding to CD3ε/δ with K_{D} values in the in low nM range, reaching from values of 1.5 nM up to 12.6 nM for human CD3ε/δ and 1.3 nM to 11.3 nM for cynomolgus CD3ε/δ. As intended, the affinity of the binding to human CD3ε/δ of the optimized anti-CD3 antibodies is decreased as compared to CD3_{orig} (in a range of between 7.5 and 63 fold) measured under same conditions by SPR.

TABLE 5
Affinity of anti-CD3 antibodies (in TCB format)
to human and cynomolgus CD3ε/δ.

| | | kₒₙ | k_{off} | t_{1/2} | K_{D} |
|---|---|---|---|---|---|
| Sample | Ligand | (1/Ms) | (1/s) | (s) | (nM) |
| P1AI7912 | human CD3 | 5.09E+06 | 6.40E−02 | 11 | 12.6 |
| | cyno CD3 | 5.59E+06 | 6.26E−02 | 11 | 11.3 |
| P1AI7913 | human CD3 | 5.60E+06 | 8.54E−03 | 81 | 1.5 |
| | cyno CD3 | 6.73E+06 | 8.51E−03 | 81 | 1.3 |
| P1AI7915 | human CD3 | 2.03E+06 | 2.31E−02 | 30 | 11.5 |
| | cyno CD3 | 2.32E+06 | 2.24E−02 | 31 | 9.7 |
| P1AH4929 | human CD3 | 5.56E+06 | 1.31E−03 | 529 | 0.2 |
| CD3_{orig} | cyno CD3 | 6.80E+06 | 1.34E−03 | 519 | 0.2 |

### Example 4-Binding of Optimized Anti-CD3 Antibodies to CD3 on Jurkat Cells

Binding to CD3 on the human reporter T-cell line Jurkat NFAT was determined by FACS for the optimized CD3 binder and the original CD3 binder CD3_{orig}, both in the TCB format.

Jurkat NFAT reporter cells (GloResponse Jurkat NFAT-RE-luc2P; Promega #CS176501) are a human acute lymphatic leukemia reporter cell line with a NFAT promoter, expressing human CD3. The cells were cultured in advanced RPMI 1640, 2% FCS, 1% Glutamax at 0.1-0.5 mio cells per ml. A final concentration of 200 μg per ml hygromycin B was added whenever cells were passaged.

For the binding assay, Jurkat NFAT cells were harvested, washed with PBS and resuspended in PBS. The antibody staining was performed in a 96-well round bottom plate. Therefore 200′000 cells were seeded per well. The plate was centrifuged for 4 min at 400× g and the supernatant was removed. The test antibodies were diluted in PBS and 25 μl of the antibody solution were added to the cells for 45 min at 4° C. To remove unbound antibody, the cells were washed twice with PBS before addition of the diluted secondary antibody (PE-conjugated AffiniPure F(ab′)2 Fragment goat anti-human IgG Fcγ Fragment Specific; Jackson ImmunoResearch #109-116-170). After 30 min incubation at 4° C. unbound secondary antibody was washed away. Before measurement the cells were resuspended in 125 μl FACS buffer and then analyzed by flow cytometry using a BD Fortessa device.

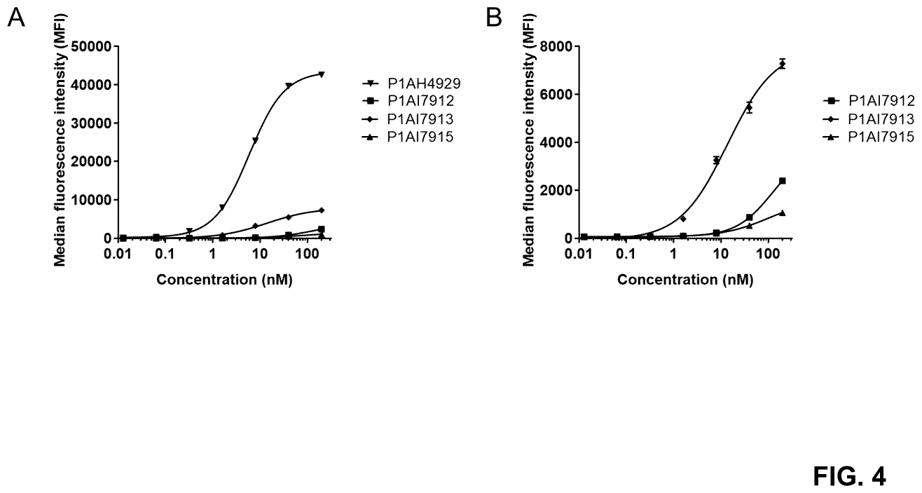

As shown in FIG. 4A, the HER2-TCBs with the optimized CD3 binders had a weaker binding to CD3 on Jurkat cells than the TCB with the original CD3 binder CD3_{orig}. The optimized CD3 binders still show significant and dose dependent binding to CD3 on Jurkat cells, however, as shown in FIG. 4B.

### Example 5-Functional Activity of Optimized Anti-CD3 Antibodies as TCBs

The functional activity of the optimized CD3 binders was tested in a Jurkat reporter cell assay and compared to the activity of the original CD3 binder CD3_{orig}. The HER2-TCBs containing either the optimized CD3 binders or the original CD3 binder CD3_{orig} were tested in the Jurkat NFAT reporter cell assay in the presence of HER2 positive KPL-4 (breast adenocarcinoma cell line, Kawasaki Medical School) and MKN-45 (gastric adenocarcinoma, DSMZ) cells. KPL-4 cells have a high expression level of HER2 and MKN-45 have a low expression level of HER2.

The Jurkat NFAT reporter cells are as described in Example 4.

Upon simultaneous binding of HER2-TCB to HER2 positive target cells and CD3 antigen (expressed on Jurkat-NFAT reporter cells), the NFAT promoter is activated and leads to expression of active firefly luciferase. The intensity of luminescence signal (obtained upon addition of the luciferase substrate) is proportional to the intensity of CD3 activation and signaling and can be measured as an activation marker. Jurkat-NFAT reporter cells grow in suspension and were cultured in advanced RPMI 1640, 2% FCS, 1% Glutamax at 0.1-0.5 mio cells per ml, 200 μg per ml hygromycin. For the assay, tumor cells were harvested and viability determined using ViCell (Beckman Coulter). 10′000 target cells/well in 10 μl medium were plated in a flat-bottom, white-walled 384-well-plate (Falcon Corning #353988) and incubated overnight at 37° C. On the next day, Jurkat-NFAT reporter cells were harvested and viability assessed using ViCell. Cells were resuspended at 0.5 mio cells/ml in cell culture medium without hygromycin B. Then, 4 ml of GloSensor cAMP reagent (Promega #E1291) was added to the cell suspension (2% of final volume) and added to tumor cells at 20′000 cells/well (20 μl/well) to obtain a final effector-to-target (E:T) ratio of 2:1. Subsequently, 20 μl/well of diluted antibodies or medium (for controls) were added to the wells. Cells were incubated for 5 h at 37° C. in a humidified incubator. At the end of incubation time, luminescence was detected using TECAN Spark 10M reader.

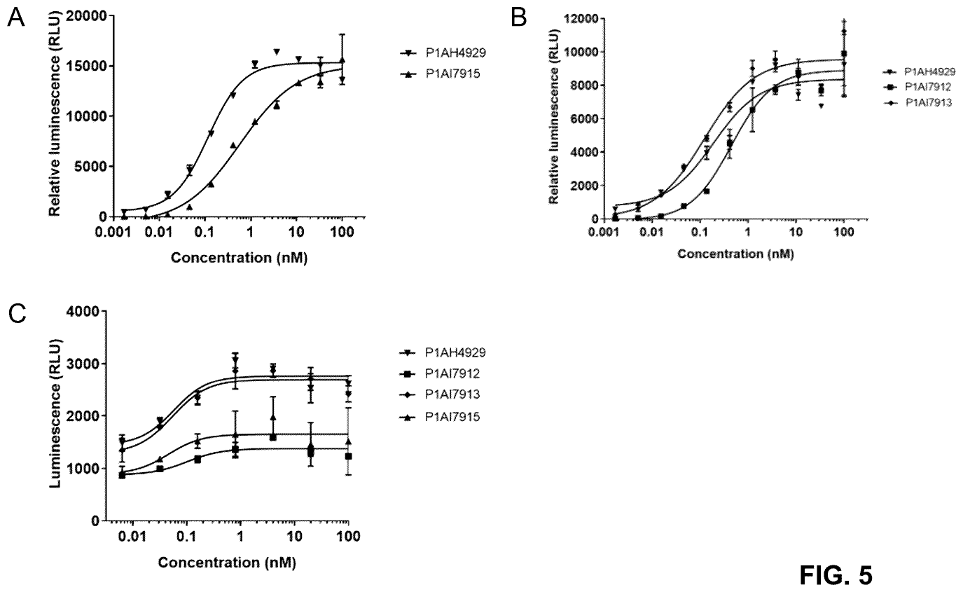

The HER2-TCB containing the optimized CD3 binder P1AI7913 has a similar functional activity as the HER2-TCB with the CD3_{orig} binder. The other two HER2-TCBs with the optimized CD3 binders have a lower functional but still relevant activity on the Jurkat NFAT reporter cells (FIGS. 5A-5C) and especially on tumor cell lines with medium to high expression level, these CD3 binders are still very potent.

### Example 6-Tumor Cell Killing with Optimized Anti-CD3 Antibodies as TCBs

In a next step, all TCB molecules were tested in a tumor cell killing assay with human isolated PBMCs (Biomex), co-incubated with either KPL-4 (breast adenocarcinoma cell line, Kawasaki Medical School) or MKN-45 (gastric adenocarcinoma, DSMZ) tumor cell lines. Tumor cell lysis was determined by LDH release after 48 h. Activation of CD4 and CD8 T cells was analyzed by upregulation of CD69 and CD25 on both cell subsets after 48 h.

Briefly, target cells were harvested with Trypsin/EDTA, washed, and plated at a density of 25′000 cells/well using flat-bottom 96-well plates. Cells were left to adhere overnight. On the next day, peripheral blood mononuclear cells (PBMCs) from a healthy donor were thawed, washed and counted (ViCell) and stored in RPMI1640 medium containing 2% FBS and 2% Glutamax (Gibco #35050-038) at room temperature until further use. For the killing assay, the antibodies were added at the indicated concentrations in triplicates. PBMCs were added to target cells at the final effector-to-target (E:T) ratio of 10:1. Target cell killing was assessed after 48 h of incubation at 37° C., 5% CO₂ in a humidified incubator by quantification of LDH released into cell supernatants by apoptotic/necrotic cells (LDH detection kit, Roche Applied Science #11 644 793 001). Maximal lysis of the target cells (=100%) was achieved by incubation of target cells with 1% Triton X-100. Minimal lysis (=0%) refers to target cells co-incubated with effector cells without bispecific antibody.

Activation of CD8 and CD4 T cells upon T cell killing of target cells mediated by the TCB was assessed by flow cytometry using antibodies recognizing the T cell activation markers CD25 (late activation marker) and CD69 (early activation marker). After 48 h incubation, PBMCs were transferred to a round-bottom 96-well plate, centrifuged at 350×g for 5 min and washed twice with PBS. For live/dead staining 25 μl/well zombie NIR (Biolegend #423105) were added per well and incubated for 45 min at 4° C. Cells were washed twice with 150 μl/well PBS. Surface staining for CD4 PE-Cy7 (BD Bioscience #557852), CD8 BV421 (Biolegend #301036), CD25 PE (BioLegend #356104) and CD69 APC (BioLegend #310910) was performed according to the suppliers' indications. Cells were washed twice with 150 μl/well PBS and fixed for 15 min at 4° C. using 100 μl/well fixation buffer (4% PFA solution). After centrifugation, the samples were resuspended in 150 μl/well FACS buffer. Samples were analyzed at BD FACS Fortessa.

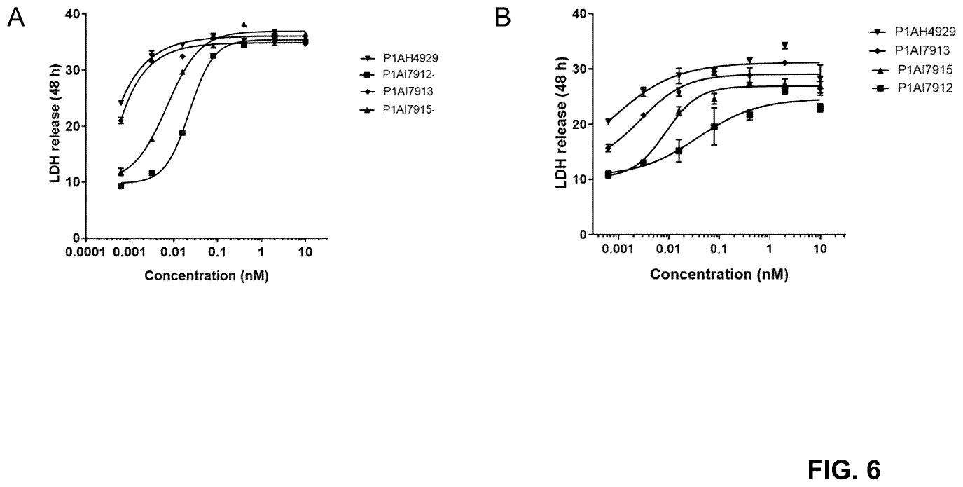

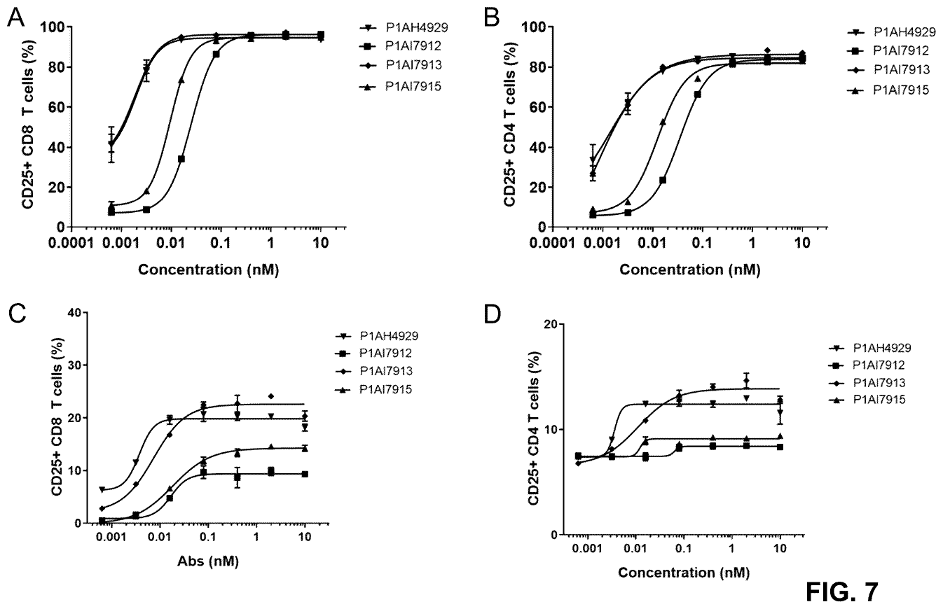

In line with the functional activity data obtained from the Jurkat NFAT reporter cell assay, the HER2-TCB with the optimized CD3 binder P1AI7913 induces similar tumor cell killing and T cell activation compared to the HER2-TCB with the CD3_{orig} binder. The other two HER2-TCBs with the optimized CD3 binders have a reduced functional activity seen for tumor cell killing as well as T cell activation (FIGS. 6A and 6B and FIGS. 7A-7D). The TCBs with these lower affinity CD3 binders are still very potent, however, and are able to induce T cell activation and tumor cell killing also on cell lines with low target expression. Therefore it can be expected that TCBs with these CD3 binders will also have in vivo efficacy.

### Example 7-In Silico MHC-II Binding Scores of Optimized Anti-CD3 Antibodies

We used NetMHCIIpan 4.0 in order to predict the number of MHC-II binding peptides in the variable regions (VH and VL) of the optimized CD3 binders. For this, we predicted binding to the nine common HLA-DRB1 alleles HLA-DRB1*01:01, HLA-DRB1*03:01, HLA-DRB1*04:01, HLA-DRB1*0701, HLA-DRB1*08:01, HLA-DRB1*09:01, HLA-DRB1*11:01, HLA-DRB1*13:01, HLA-DRB1*15:01, with a “strong binder” threshold of Percentile Rank 1, and a “weak binder” threshold of Percentile Rank 5. The results are shown in Table 6. A lower number of MHC-II binding peptides in the sequence (n_total), as seen for the optimized CD3 binders, might lead to a favorable immunogenicity profile in humans. By the same token, we could also reduce the number of MHC-II binding peptides in the “strong” Percentile Rank range (n_strong), which comprises peptides predicted to have a very high affinity for the considered MHC-II alleles and thereby a high likelihood of being presented as peptide-MHC II complexes on cells. As indicated in Table 6, the optimized CD3 binders did not contain any strong MHC-II binding peptides.

TABLE 6
MHC-II binding scores predicted by NetMHCIIpan.

| | Fv | Fv | Fv |
|---|---|---|---|
| Binder | n_strong | n_weak | n_total |
| CD3_{orig} | 12 | 66 | 78 |
| P1AI7912 | 0 | 31 | 31 |
| P1AI7913 | 0 | 38 | 38 |
| P1AI7915 | 0 | 34 | 34 |

In order to subtract MHC-II binding peptides with a high probability of being non-antigenic, we did not count peptides where the MHC-II binding 9mer core is occurring in 10 or more human V region germlines. Hence, the above table only counts peptides which are either non-germline in the 9mer core, or where the 9mer core is occurring only in a very small number of human germlines.

### Example 8-Hydrophobic and Charged-Patch Volume of Optimized Anti-CD3 Antibodies Calculated from X-Ray Structure and Homology Models

One focus of the rational design attempt was to reduce the size of hydrophobic and positively charged patches in the variable region of the CD3 binders in order to reduce the rate of nonspecific uptake by cells and in general to improve the biophysical properties of the molecules.

Using X-ray structures or homology models of the Fv region with capped C-termini, protonation states at pH 7.4 were assigned with PROPKA (an in silico method for the prediction of the pKa values of ionizable residues in proteins; Bas et al., Proteins: Structure, Function, and Bioinformatics, 73(3), 765-783 (2008)), and the 3D charge distribution was calculated with APBS (Adaptive Poisson-Boltzmann Solver software, solving the equations of continuum electrostatics for biomolecules and used to study intra- and intermolecular electrostatic interactions; Jurrus et al., Protein Science 27(1), 112-128 (2018)) at 150 mM ionic strength. The volume integral of all positive and negative voxels is reported as the patch score in Table 7.

TABLE 7
Positive and negative charged patch volume of
optimized CD3 binders volume integral of all
positive and negative voxels).

| | Integral_positive | | Integral_negative | |
|---|---|---|---|---|
| | | Percent | | Percent |
| Binder | Absolute | change | Absolute | change |
| CD3_{orig} | 71031 | n/a | −20893 | n/a |
| P1AI7912* | 61004 | −14.12% | −23055 | +10.35% |
| P1AI7913* | 66812 | −5.94% | −23649 | +13.19% |
| P1AI7915* | 57160 | −19.53% | −24423 | +16.00% |

*Homology model instead of X-ray crystal structure was used

For the optimized CD3 binders, the size of the positive charge volume integral decreases by up to almost 20%, while the size of the negative charge volume integral increases (by approximately 10 to 16%).

The in silico measure for hydrophobicity was calculated with a method that is an implementation of Spatial Aggregation Propensity (SAP) (Chennamsetty et al., PNAS 106, 11937-11942 (2009)) using the Wimley-White hydrophobicity scale (Wimley and White, Nat Struct Mol Biol 3, 842-848 (1996)). The results are listed in Table 8. Lower values indicate less hydrophobic character.

TABLE 8
Hydrophobicity values of optimized CD3 binders.

| | SAP VH | | SAP VL | |
|---|---|---|---|---|
| | | Percent | | Percent |
| Binder | Absolute | change | Absolute | change |
| CD3_{orig} | 35.16 | n/a | 23.38 | n/a |
| P1AI7912* | 35.35 | +0.54% | 17.76 | −24.04% |
| P1AI7913* | 34.04 | −3.18% | 18.05 | −22.80% |
| P1AI7915* | 32.99 | −6.17% | 17.29 | −26.05% |

*Homology model instead of X-ray crystal structure was used

For the VH domain of the optimized CD3 binders P1AI7913 and P1AI7915 there is a slight reduction of the hydrophobic patch score, but apart from that, the reduction in hydrophobic character seems to be driven by the VL domain (which is the same for P1AI7912, P1AI7913 and P1AI7915).

### Example 9-Polyreactivity Testing of Optimized Anti-CD3 Antibodies

Polyreactivity was tested using an ELISA-based read out. A set of different antigens depicted in Table 9 was coated on a Nunc MaxiSorp microtiter plate (Fisher Scientific) overnight at 4° C. with a concentration of 1 μg/ml in Dulbecco's phosphate buffered saline (DPBS, PAN Biotech #P04-36500) each. After washing the plate two times using PBS (Roche Diagnostics #11666789001) containing 0.1% Tween-20, plates were blocked with DPBS, 2% bovine serum albumin (BSA, Roche Diagnostics #10735108001) for 1 hour at room temperature. After washing the plate three times, the HER2 TCBs were added with a concentration of 1 μg/ml diluted in DPBS and incubated for 1 hour at room temperature. Subsequently, plates were washed three times and an anti-human IgG F(ab′)2-HRP (Jackson ImmunoResearch #109-036-006) diluted 1:5000 in PBS, 0.5% BSA, 0.05% Tween-20 was added. After incubation for 1 hour at room temperature and washing for four times, TMB substrate (Sigma-Aldrich #T0440) was added and absorption was measured at 370/492 nm.

As desired, none of the molecules showed a significant sticky/polyreactive binding profile.

TABLE 9
Reactivity to different antigens by optimized CD3 binders.

| | Absorbance (OD) | | | |
|---|---|---|---|---|
| Antigen | CD3_{orig} | P1AI7912 | P1AI7913 | P1AI7915 |
| Histone H4 aa2-24 | 0.05 | 0.00 | 0.06 | 0.07 |
| Parathyroid Hormone | −0.01 | 0.00 | 0.00 | 0.00 |
| Streptavidin | 0.00 | 0.00 | 0.01 | 0.00 |
| Hemocyanin | 0.02 | 0.02 | 0.07 | 0.08 |
| Bovine Serum Albumin | 0.00 | 0.00 | 0.01 | 0.01 |
| Insulin | 0.01 | 0.02 | 0.04 | 0.04 |
| huCD40 | 0.23 | 0.24 | 0.27 | 0.31 |
| Apolipoprotein A-1 | 0.00 | 0.01 | 0.01 | 0.01 |
| HSP90 | 0.02 | 0.07 | 0.03 | 0.07 |
| Cardiolipin | −0.01 | 0.00 | 0.01 | 0.01 |
| Heparin | −0.02 | −0.01 | −0.01 | −0.01 |
| DNA | −0.01 | −0.01 | 0.03 | 0.01 |
| E. coli Lysate | 0.00 | 0.05 | 0.03 | 0.07 |
| Gelatine (2%) | −0.01 | −0.01 | 0.01 | 0.01 |
| human CD3ε | 2.87 | 2.20 | 2.83 | 2.99 |

### Example 10-Nonspecific Cell Binding of Optimized Anti-CD3 Antibodies

An ELISA was performed to evaluate the “stickiness” of the optimized CD3 binders by measuring the specific and unspecific binding on cells.

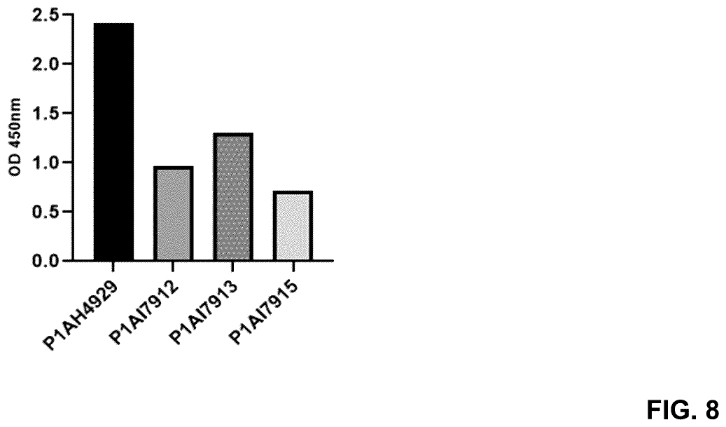

This ELISA was established to detect unspecific binding of anti-CD3-antibodies of different formats to adherent cells. Therefore, adherent cells without CD3 expression on their surface (e.g. CHO-K1) were used and binding was measured. Cells were seeded over night in growth medium, antibodies were added and incubated for 2 h at room temperature. The cells were carefully washed with PBS, fixed with glutaraldehyde, and washed again with PBS/2% FBS. Bound antibodies were detected using a F (ab) 2 fragment goat <human Fcγ fragment>POD (peroxidase), incubation at room temperature. After washing steps with PBS/2% FBS, the substrate TMB (3,3′,5,5′ tetramethylbenzidine) was added and the enzymatic reaction was stopped after 10 min using H₂SO₄. The amounts of bound antibodies were measured by detecting the absorbances in correlation to the antibody concentrations at 450 nm with an Tecan Infinite reader. The result (for an antibody concentration of 200 nM) is shown in FIG. 8. The signal correlated to the non-specific binding to target (i.e. CD3)-negative cells, the optimized CD3 binders P1AI7912, P1AI7913 and P1AI7915 show reduced stickiness in this experiment (% of signal as compared to parental antibody PIAH4929 (CD3_{orig}): P1AI7912 (40%), P1AI7913 (54%), P1AI7915 (30%)

### Example 11-In Vitro PK Parameters-Nonspecific Cellular Accumulation (LUCA)

Antibodies containing extensive positively charged patches on their surface, particularly in their Fv region, show elevated non-specific clearance in vivo (see e.g. Kraft et al., mAbs 12, 1683432 (2020), Liu et al., mAbs 13, 1993769 (2021)). Measurement of cellular accumulation rates using the LUCA assay in human primary liver sinusoidal endothelial cells can be used to estimate the nonspecific clearance of therapeutic antibodies.

The LUCA assay makes use of a pH dependent difference in fluorescence intensity of a fluorophore covalently attached to the antibody construct of interest during the in vitro procedure. The construct with the attached dye is then incubated with human primary liver sinusoidal endothelial cells which express physiological levels of human FcRn. The fluorescence in the endothelial cell remains low if the labeled construct shows low endocytosis rates and is well recycled by the FcRn in the endothelial cell, but increases if the labeled construct shows high endocytosis driven uptake and is not well recycled and is processed in late endosomes and degraded in the lysosome, since the pH is decreasing in these late endosomes and the lysosome. The higher the LUCA value, the higher may be the in vivo clearance. Details to the approach can be found in patent application WO 2021/204743 (incorporated herein by reference).

Briefly, data is acquired by labeling the antigen binding molecules with a pH-sensitive dye exhibiting high fluorescence when accumulating in the lysosome (acidic pH 5.5) and low fluorescence when remaining outside the cell (neutral pH 7.4). Human or animal endothelial cells are incubated with labeled antibodies for 2 and 4 hours and the fluorescent readout is recorded using a flow cytometer. The geo-mean intensities are used for linear regression analysis. The extracted slopes form, when normalized to standard antibodies, the so-called normalized non-specific cellular accumulation rate.

Antibody Labeling: The Antibodies were labeled using the SiteClick™ Antibody Azido Modification Kit (Thermo Fisher Scientific) according to the manufactures instructions. Briefly, N-linked galactose residues of the Fc-region were removed by β-galactosidase and replaced by an azide-containing galactose (GalNaz) via β-1,4-galactosyltransferase (GalT). This azide modification enables a copper-free conjugation of sDIBO-modified dyes. The pH-sensitive amine-reactive dye (523 nm) was purchased from Promega and coupled to a sulfo DBCO PEG4 amine. Antibodies were labeled with a molar dye excess of 2. Excess dye was removed using the Amicon® Ultra-2 Centrifugal Filter with a MWCO of 50 kD (EMD Millipore, #UFC200324) and antibodies were re-buffered in 20 mM histidine buffer (pH 5.5). The concentration of the labeled antibodies [1] as well as the dye to antibody ratio (DAR) [2] was determined with a Nanodrop spectrometer at 280 nm and 532 nm.

$$CAB = [ A 280 nm - [ A 280 nm * CFDye ] ] / ε mA b \qquad [ 1 ]$$

$$DAR = [ A 532 nm * MW mA b ] / [ cm Ab * εDye ] \qquad [ 2 ]$$

$$εDye = 47225 \; 15 CFDye = 0.36$$

Cell Maintenance and Preparation: Cryopreserved human liver-derived endothelial cells (HLEC-P2) were purchased from Lonza (Lonza, #HLECP2). Cell were maintained in EBM Endothelial Cell Growth Basal Medium-2 (Lonza, #CC-3156) supplemented with EGM-2 MV (Lonza, #CC-4176). Five days prior antibody treatment, cells were plated onto collagen I coated 100 mm culture dishes (Corning® BioCoat™, #354450) and two days prior treatment sub-cultured into collagen I coated 96-well plates (Corning® BioCoat™, #354407) at a cell density of 4×104. On the day of the experiment, cells were washed twice with 200 μl pre-warmed medium and subsequently incubated with 400 nM labeled antibody or 20 mM histidine buffer (pH 5.5) as negative control in medium. After 2 and 4 hours, the antibody solution was removed and cells were washed once with 200 μl ice-cold DPBS (without Mg and Ca) and detached by applying 100 μl Trypsin (with EDTA) for 2.5 minutes at 37° C. Trypsin was inactivated by the addition of 100 μl FACS Buffer (20% FCS, 1 mM EDTA in DPBS).

Quality control: Biophysical binding properties are key determinants affecting clearance mechanisms. Therefore, it was important to assess whether the binding affinities of the antibodies changed during the labeling process. Heparin chromatography and neonatal Fc receptor binding has been previously shown to predict antibody clearance in vitro (Kraft et al., mAbs 12, 1683432 (2020)). Herein, this method was used to account for potential aberrant binding properties introduced by the click label. To confirm the absence of unbound dye and to verify the concentration measured at the spectrometer, a size exclusion chromatography of the labeled antibodies was performed. Samples were separated using a BioSuite Diol (OH) column (Waters, #186002165) with a potassium dihydrogen phosphate buffer (pH 6.2) as the mobile phase at a flow rate of 0.5 ml/min. Detectors at 280 nm and 532 nm were used to quantify and analyze the labeled antibodies. The area under the curve (AUCs) at 280 nm and 532 nm was extracted to calculate the concentration. The geo-mean of the AUCs from all antibodies was computed and the deviation from each antibody to this geo-mean was identified. For an antibody to be reliable within this assay, the difference from the geo-mean was expected to be below 15%.

Flow Cytometry and Pharmacokinetic Analysis: The mean fluorescent intensity (MFI, more specifically the geometric mean (geo-mean)) of the internalized antibodies was acquired using the MACSQuant® Analyzer 10 (Miltenyi Biotec) equipped with a laser to excite at 488 nm and a filter to collect emitted light at 585 nm/540 nm. The exact same conditions, gains and gates were used for both times points (2 hours and 4 hours). Data extraction was performed using the FloJo_V10 software. Values of the negative control was subtracted from all geo-mean values followed by normalization to the DAR. The normalized geo-mean values from each antibody were plotted as linear regression curve using GraphPad Prism to extract the slope (Geo Mean MFI/min for 120 and 240 min). Two standard antibodies were selected to normalize the slopes:

Motavizumab-YTE (G. J. Robbie et al., Antimicrob. Agent Chemother. 57 (12), 6147 (2013)) was set to 0 and CD20-CD3 TCB (Hutchings et al., Blood 134, 2871 (2019)) was set to 1.

Testing of CD3 binders: By reducing the size of positively charged patches in the variable region of CD3_{orig}, the successfully engineered CD3 binders should show superior pharmacokinetic properties including reduced nonspecific clearance.

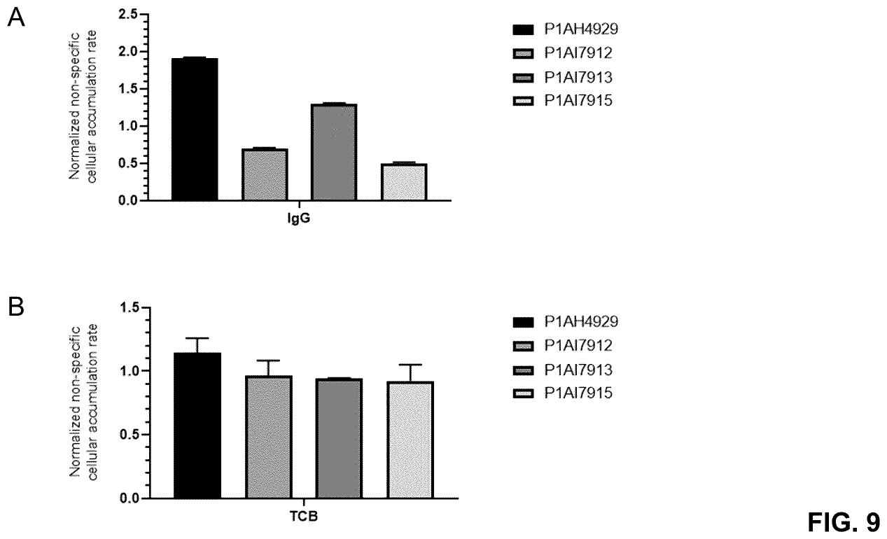

The parental and optimized CD3 binders were first compared in a bivalent IgG-like format (in contrast to the TCB format, where they are present monovalently in combination with a bivalent HER2 binder). Here, we could evaluate the effect of the positive patch removal in isolation without the additional effect of the HER2 binders. FIGS. 9A and 9B show a strongly reduced cellular accumulation rate for the IgG versions of the optimized CD3 binders (P1AH7912, P1AH7913, P1AH7915) compared to the parental CD3_{orig} (PIAH4929). This finding is further supported by the strong correlation between the results of this in vitro study and the percent change in volume integral of all positive voxels (Example 8, Table 7) with P1AI7913 showing the lowest reduction in positive charge patch volume and the lowest reduction in cellular accumulation rate compared to the parental CD3 binder.

Furthermore, this assay was used to identify TCBs containing CD3 binders with reduced cellular accumulation rate compared to the original CD3_{orig} plus HER2 binders in a bivalent configuration and rank them by this property. Here, all three optimized CD3 binders showed a reduced non-specific cellular accumulation rate compared to the parental CD3_{orig} binder. However, the differences between the individual optimized CD3 binders in IgG format did not translate directly to the TCB format indicating similarly improved pharmacokinetic properties compared to the parental CD3 binder (9B). The differences in the observation between IgG format and TCB format can be attributed to the fact that the non-specific clearance properties of the molecule (normalized non-specific cellular accumulation) are a net-sum of the individual binder (Fabs) properties (i.e. an additive effect of all binders). As the TCB format contains two target binders and only one CD3 binder, while the IgG format contains two CD3 binders and no target binders, the effect of CD3 binder engineering will be much stronger in the IgG format compared to the TCB format where the PK properties are also affected by the two target binder properties. Therefore, for improving pharmacokinetic properties of a TCB, the properties of all three binders need to be considered. In this case, we could show the improved properties of the optimized CD3 binders both in the IgG format (more pronounced) and in the TCB format (less pronounced due to the additional influence of two target binders, but still relevant).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An antibody that binds to CD3, wherein the antibody comprises a first antigen binding domain, comprising
(i) a heavy chain variable region (VH) comprising a heavy chain complementarity determining region (HCDR) 1 of SEQ ID NO: 1, a HCDR 2 of SEQ ID NO: 9, SEQ ID NO: 13 or SEQ ID NO: 15, and a HCDR 3 of SEQ ID NO: 3,
and
(ii) a light chain variable region (VL) comprising a light chain complementarity determining region (LCDR) 1 of SEQ ID NO: 5, a LCDR 2 of SEQ ID NO: 11 and a LCDR 3 of SEQ ID NO: 7.

2. The antibody of claim 1, wherein the VH of the first antigen binding domain comprises an amino acid sequence that is at least about 95%, 96%, 97%, 98%, 99% or 100% identical to an amino acid sequence selected from the group consisting of SEQ ID NO: 10, SEQ ID NO: 14, SEQ ID NO: 16, SEQ ID NO: 17, SEQ ID NO: 18 and SEQ ID NO: 19, and/or the VL of the first antigen binding domain comprises an amino acid sequence that is at least about 95%, 96%, 97%, 98%, 99% or 100% identical to the amino acid sequence of SEQ ID NO: 12.

3. An antibody that binds to CD3, wherein the antibody comprises a first antigen binding domain comprising a VH sequence selected from the group consisting of SEQ ID NO: 10, SEQ ID NO: 14, SEQ ID NO: 16, SEQ ID NO: 17, SEQ ID NO: 18 and SEQ ID NO: 19, and a VL sequence of SEQ ID NO: 12.

4. The antibody of any one of claims 1 to 3, wherein the first antigen binding domain is a Fab molecule.

5. The antibody of any one of claims 1 to 4, comprising an Fc domain composed of a first and a second subunit.

6. The antibody of any one of claims 1 to 5, comprising a second and optionally a third antigen binding domain that binds to a second antigen.

7. The antibody of claim 6, wherein the second and/or, where present, the third antigen binding domain is a Fab molecule.

8. The antibody of any one of claims 1 to 7, wherein the first antigen binding domain is a Fab molecule wherein the variable domains VL and VH or the constant domains CL and CH1, particularly the variable domains VL and VH, of the Fab light chain and the Fab heavy chain are replaced by each other.

9. The antibody of any one of claims 6 to 8, wherein the second and, where present, the third antigen binding domain is a conventional Fab molecule.

10. The antibody of any one of claims 6 to 9, wherein the second and, where present, the third antigen binding domain is a Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

11. The antibody of any one of claims 6 to 10, wherein the first and the second antigen binding domain are fused to each other, optionally via a peptide linker.

12. The antibody of any one of claims 6 to 11, wherein the first and the second antigen binding domain are each a Fab molecule and either (i) the second antigen binding domain is fused at the C-terminus of the Fab heavy chain to the N-terminus of the Fab heavy chain of the first antigen binding domain, or (ii) the first antigen binding domain is fused at the C-terminus of the Fab heavy chain to the N-terminus of the Fab heavy chain of the second antigen binding domain.

13. The antibody of any one of claims 6 to 12, wherein the first, the second and, where present, the third antigen binding domain are each a Fab molecule and the antibody comprises an Fc domain composed of a first and a second subunit; and wherein either (i) the second antigen binding domain is fused at the C-terminus of the Fab heavy chain to the N-terminus of the Fab heavy chain of the first antigen binding domain and the first antigen binding domain is fused at the C-terminus of the Fab heavy chain to the N-terminus of the first subunit of the Fc domain, or (ii) the first antigen binding domain is fused at the C-terminus of the Fab heavy chain to the N-terminus of the Fab heavy chain of the second antigen binding domain and the second antigen binding domain is fused at the C-terminus of the Fab heavy chain to the N-terminus of the first subunit of the Fc domain; and the third antigen binding domain, where present, is fused at the C-terminus of the Fab heavy chain to the N-terminus of the second subunit of the Fc domain.

14. The antibody of any one of claims 5 to 13, wherein the Fc domain is an IgG, particularly an IgG₁, Fc domain.

15. The antibody of any one of claims 5 to 14, wherein the Fc domain is a human Fc domain.

16. The antibody of any one of claims 5 to 15, wherein the Fc comprises a modification promoting the association of the first and the second subunit of the Fc domain.

17. The antibody of any one of claims 5 to 16, wherein the Fc domain comprises one or more amino acid substitution that reduces binding to an Fc receptor and/or effector function.

18. The antibody of any one of claims 6 to 17, wherein the second antigen is a target cell antigen, particularly a tumor cell antigen.

19. The antibody of any one of claims 6 to 18, wherein the second antigen is HER2.

20. The antibody of claim 19, wherein the second and, where present, the third antigen binding domain comprises a VH comprising a HCDR 1 of SEQ ID NO: 20, a HCDR 2 of SEQ ID NO: 21, and a HCDR 3 of SEQ ID NO: 22, and a VL comprising a LCDR 1 of SEQ ID NO: 24, a LCDR 2 of SEQ ID NO: 25 and a LCDR 3 of SEQ ID NO: 26.

21. The antibody of claim 19 or 20, wherein the second and, where present, the third antigen binding domain comprises a VH comprising an amino acid sequence that is at least about 95%, 96%, 97%, 98%, 99% or 100% identical to the amino acid sequence of SEQ ID NO: 23, and/or a VL comprising an amino acid sequence that is at least about 95%, 96%, 97%, 98%, 99% or 100% identical to the amino acid sequence of SEQ ID NO: 27.

22. An isolated polynucleotide encoding the antibody of any one of claims 1 to 21.

23. A host cell comprising the isolated polynucleotide of claim 22.

24. A method of producing an antibody that binds to CD3, comprising the steps of (a) culturing the host cell of claim 23 under conditions suitable for the expression of the antibody and optionally (b) recovering the antibody.

25. An antibody that binds to CD3 produced by the method of claim 24.

26. A pharmaceutical composition comprising the antibody of any one of claim 1-21 or 25 and a pharmaceutically acceptable carrier.

27. The antibody of any one of claim 1-21 or 25 or the pharmaceutical composition of claim 26 for use as a medicament.

28. The antibody of any one of claim 1-21 or 25 or the pharmaceutical composition of claim 26 for use in the treatment of a disease.

29. The antibody or pharmaceutical composition for use of claim 28, wherein the disease is cancer.

30. Use of the antibody of any one of claim 1-21 or 25 or the pharmaceutical composition of claim 26 in the manufacture of a medicament.

31. Use of the antibody of any one of claim 1-21 or 25 or the pharmaceutical composition of claim 26 in the manufacture of a medicament for the treatment of a disease.

32. The use of claim 31, wherein the disease is cancer.

33. A method of treating a disease in an individual, comprising administering to said individual an effective amount of the antibody of any one of claim 1-21 or 25 or the pharmaceutical composition of claim 26.

34. The method of claim 33, wherein the disease is cancer.

35. The invention as described hereinbefore.
